(19) United States
(12) Patent Application Publication
(10) Pub. No.: US 2025/0000743 A1
(43) Pub. Date: Jan. 2, 2025

(54) SYSTEMS AND METHODS FOR TREATING FLUID OVERLOAD

(71) Applicant: AQUAPASS LTD, Or-Akiva (IL)

(72) Inventor: Yaacov Nitzan, Hertzelia (IL)

(21) Appl. No.: 18/699,539

(22) PCT Filed: Oct. 7, 2022
(86) PCT No.: PCT/IB2022/000577

§ 371 Date: Apr. 8, 2024

Related U.S. Application Data
(60) Provisional application No. 63/253,991, filed on Oct. 8, 2021.

(30) Foreign Application Priority Data
Oct. 8, 2021 (US) 63253991

(51) Int. Cl. A61H 36/00; G16H 10/60; G16H 20/30

(52) U.S. Cl. CPC A61H 36/00; G16H 10/60; G16H 20/30; A61H 2201/0103; A61H 2201/0207; A61H 2201/0242; A61H 2201/1215; A61H 2201/165; A61H 2201/168; A61H 2201/1697; A61H 2201/501; A61H 2201/5043; A61H 2201/5064; A61H 2201/5082; A61H 2201/5084; A61H 2201/5087; A61H 2230/065; A61H 2230/305; A61H 2230/505; A61H 2230/625

(57) ABSTRACT

The invention generally relates to systems and methods for treating fluid overload in a patient by providing controlled stimulation of fluid transfer directly and non-invasively from an interstitial compartment of the patient through the skin of the patient.

### CROSS-REFERENCE TO RELATED APPLICATIONS

This application claims priority to, and the benefit of, U.S. Provisional Application No. 63/253,991, filed on Oct. 8, 2021, the content of which is incorporated by reference herein in its entirety.

### FIELD OF THE INVENTION

The invention generally relates to systems and methods for treating fluid overload in a patient by providing controlled stimulation of fluid transfer directly and non-invasively from an interstitial compartment of the patient through the skin of the patient.

### BACKGROUND

Some patients suffer from chronic pathological conditions in which there is an imbalance of fluids in the body. For example, edema is a medical condition in which fluids flow from the intravascular to the interstitial compartment at a rate that exceeds the removal rate leading to accumulation of excessive fluids in the interstitial compartment. Accordingly, edema is an important contributor to heart failure symptoms. Congestive heart failure (CHF), for example, occurs when fluid builds up in the extracellular compartments (intravascular and interstitial).

Because of the excess fluid, the heart is unable to pump sufficiently to maintain blood flow to meet the body's needs. A person suffering from heart failure may experience shortness of breath, exhaustion, rapid heartbeat, and swollen limbs. Heart failure is a common, potentially fatal condition, and is a leading cause of hospitalization in people over 65 years of age.

In heart failure, the heart works harder to eject blood, leading to a buildup of blood pressure. When elevated blood pressure prevents draining from the interstitial compartments of the body, edema, or swelling caused by fluid accumulation in bodily tissues, may occur. The additional work of the heart due to edema weakens the heart, further reducing the ability of the heart to function properly. The fluid accumulation may lead to additional health conditions, hospitalization, and death.

A primary treatment goal in CHF is to achieve adequate fluid and electrolytes balance while avoiding renal dysfunction and other adverse effects. However, even when guideline-recommended therapies are implemented, many chronic heart failure patients have signs of edema, such as dyspnea and pitting edema.

Current therapies rely primarily on drugs that reduce intravascular volume and pressure, such as diuretics, to reabsorb the excessive interstitial fluids and reduce the edema. Patients suffering from such conditions are typically treated on a weekly basis in hospitals. Most of those patients develop resistance to the pharmaceutical treatments, thereby eliminating the primary treatment option. Furthermore, adequate decongestion is not achieved in many patients who rely on current pharmaceutical treatments.

Current medical options are also unable to adequately treat other pathologies that cause a buildup of fluids and electrolytes in the interstitial compartment, such as renal dysfunction, cirrhosis and lymphedema.

### SUMMARY

The present invention provides systems and methods for enhancing removal of excess bodily fluid, electrolytes, and/or nitrogen products (i.e., urea or the like) from a patient's body and though their skin via sweat as a means for treating fluid overload, electrolytic imbalances, and/or urea imbalances. In particular, the present invention includes a fluid stimulation system configured to provide controlled, non-invasive skin fluid transfer, independent and regardless of pharmaceuticals, nutrition, and mental condition of the patient.

The fluid stimulation system includes a treatment device that can be placed over one or more portions or parts of a patient's body, including, but not limited to, one or more legs, one or more arms, one or more hands or feet, torso, lower back, and abdomen up to the level of the chest, for example. The treatment device is operably coupled to a console unit configured to control one or more aspects of the device, including controlling a warm air environment provided via the treatment device to a patient for causing sweating. For example, the console unit may control at least one of temperature of the environment, flow rate of air into and out of the environment, humidity within the environment, and other additional parameters which assure safe, as well as effective, operation during use. In some embodiments, for example, the console unit may provide heated air to the treatment device by way of a heat generator and air blower or fan incorporated into the console unit.

Accordingly, upon being placed over the desired portions of the patient's body, the treatment device is able to create a controlled, homogenous warm air environment at a predetermined volume around the patient's body in order to create conditions that initiate sweat production. Throughout the treatment, fluids and components of such fluids from the interstitial compartment are removed from the body by the sweat glands, thereby decongesting fluid overloaded patients as well as treating other pathologies. In particular, sweating is one way the body releases fluids. It should be noted that the systems and methods of the present disclosure may also enhance fluid transfer through the skin, not only by way of the sweat glands, but also via hair follicles and through the skin itself (e.g., via the epidermis and stratum corneum).

There are two main types of sweat glands that differ in their structure, function, secretory product, mechanism of excretion, and anatomic distribution. One sweat pathway includes eccrine glands or major sweat glands located throughout the entire body. Eccrine sweat glands are distributed almost all over the human body, in varying densities. The water-based secretion from eccrine sweat glands represents a primary form of cooling. In particular, eccrine glands have at least two primary functions: 1) thermoregulation, in which sweat (through evaporation and evaporative heat loss) can lead to cooling of the surface of the skin and a reduction of body temperature; and 2) excretion, in which eccrine sweat gland secretion can provide a significant excretory route for water and electrolytes. Additionally, or alternatively, fluid may be removed from the body by way of apocrine glands and across the stratum corneum of the skin. Apocrine sweat glands are mostly limited to the axillae (armpits) and perineal area in humans. Sweat is 98-99% water with some electrolytes, such as sodium and chloride. Sweat also contains sodium chloride (NaCl), fatty acids, lactic acid, citric acid, ascorbic acid, and urea. Eccrine glands can secrete up to 2 L/hour of fluids directly from the interstitial compartment, and total body fluid loss from sweat can be more than 10 L/day.

Accordingly, the system of the present invention can be used to promote fluid transfer from the interstitial and intravascular compartment via stimulation of sweat, osmosis, other fluid transfer, or a combination thereof using local elevation in skin temperatures or increase in ambient osmotic pressure favoring fluid transfer from the interstitial compartment to outside the body.

The treatment device may take multiple forms. For example, in a preferred embodiment, the treatment device is in the form of a wearable device (generally resembling an article of clothing for enclosing one or more portions of a patient's body to provide the environment). For example, the treatment device may generally be in the form of overalls (also known as coveralls), in which the legs and majority of the patient's trunk or torso are covered up to the chest level, and further includes sleeves that cover the shoulders and upper arm areas of a patient, while the neck and head remain uncovered. Accordingly, the warm air environment provided by the treatment device can cover a majority of the patient's body. In alternative embodiments, the treatment device, as well as other components of the system (e.g., console unit, sensors, etc.) may be incorporated into a chair, couch, bed, or other article of furniture, thereby providing a comfortable setting in which a patient can simply sit or lay and received treatment. Such an article of furniture may include some form of cover or the like to be pulled over an exposed portion of the patient's body (i.e., portion of body that is not in contact with the furniture) to thereby aid in forming a micro-environment for the treatment.

The fluid stimulation system further includes multiple sensors that can be used in controlling the output of heated air to the device, as well as used in the continuous monitoring of the patient during a given treatment session, including monitoring of patient vital signs. For example, temperature and humidity sensors may be placed within an air flow path of the treatment device. In particular, the treatment device may include an inlet port (e.g., for receiving heated air from the console unit via a hose or other connection means) and an outlet port (e.g., for allowing heated air that has passed through portions of the treatment device to exit the treatment device). The inlet and outlet ports may generally be coupled to a framework of channels or chambers of the treatment device through which the heated air flows to provide the warm air environment within the interior of the treatment device (to which a patient is exposed). Accordingly, each of the inlet and outlet ports may include respective temperature and relative humidity sensors for monitoring temperature and relative humidity readings at the respect inlet and outlet ports.

Furthermore, the system may include one or more skin temperature sensors placed relative and in proximity to the patient's body (i.e., either in direct contact or indirect contact, such as an infrared sensor) for monitoring the skin temperature and used in verifying that the skin temperature does not, in any circumstance, elevate above a threshold value (i.e., less than 40° C., and more preferably no greater than 39° C.). The system may also include one or more accelerometers relative and in proximity to the patient's body for monitoring patient movement, specifically collecting acceleration data of the patient and used in determining a patient's pose (i.e., a supine or prone position, a sitting position, or a standing position). The console unit may also include various sensors, including a temperature sensor, a flow meter (or flow sensor), and other sensors.

The sensors of the treatment device and the console unit provide data to a controller, which is configured to process and analyze such data for controlling and monitoring conditions of the treatment device. For example, during treatment, data measurements provided by the various sensors may be provided to the controller, which, in turn, is able to control the output of the console unit. For example, in the event that the skin temperature elevates above the threshold value (i.e., the maximum temperature at which the treatment can be safely performed, which could be as high as 39° C., but ultimately less than 40° C.), the controller terminates operation of the console unit, and thus prevents warm air from entering the treatment device. In some embodiments, the controller processes temperature and relative humidity measurements collected via sensors at the inlet and outlet ports of the treatment device and, together with airflow measurements collected via a flow sensor the console unit, is able to continuously calculate a sweat rate of the patient during the procedure. Depending on the given treatment, including the specific parameters of the treatment (i.e., planned fluid removal amount and overall time for procedure), the controller is able to control output from the console unit on-the-fly based on sweat weight calculations, including adjusting flow rate and/or inflow air temperature to the treatment device until a sufficient sweat rate is achieved.

The fluid stimulation device, most notably the console unit and controller, may generally be controlled via a software-based application running either as a web-based app or running as a local app downloaded on a user's computing device. For example, a user (i.e., physician or other medical professional) may utilize a personal computing device (e.g., laptop, tablet, smartphone, etc.) to access software associated with the fluid stimulation device, in which a user interface is provided allowing for the monitoring of a given procedure, as well as subsequent control of such procedure. The computing device is configured to communicate and exchange data with the console unit and controller via a wired or wireless connection (i.e., Wi-Fi network, Bluetooth radio, Near Field Communication (NFC), etc.). The computing device may also be configured to communicate with other sensors not incorporated into either of the treatment device or console unit, including sensors for monitoring patient vital signs (i.e., heart rate, blood pressure, saturation, etc.). Accordingly, the user interface provided on a user's computing device may provide real-time data during the procedure, including, but not limited to, information associated with a current treatment procedure (i.e., elapsed time since treatment began, sensor measurement data and related calculations of the environment provided by the treatment device, including air temperature and absolute humidity, skin temperature, current sweat rate, accumulative sweat measurement, and more). Furthermore, the user interface may provide a user with input controls for controlling the given treatment, including basic start and stop controls, controls for selecting as well as control over output from the console unit (i.e., air flow rate and heat output).

Accordingly, the fluid stimulation system of the present invention provides a non-invasive, multiple use device for removing excess fluid in a patient by using external stimulation to increase sweat rates. The system provides a homogeneous warm temperature environment around a portion of the patient's body as a means of increasing skin temperature and initiating perspiration. The system is configured in such a way to ensure that the body core temperature of the patient remains within normal range. In addition, because patient's comfort is paramount with this form of therapy, the sweat evaporates instantaneously, thus avoiding the awareness of perspiration by the patient and enabling long durations of treatments, if required. Furthermore, the system design allows for use outside of a hospital, including use at an outpatient clinic or even at home, thereby increasing the ease with which treatments can be performed.

### BRIEF DESCRIPTION OF THE DRAWINGS

FIGS. 1A and 1B are diagrammatic illustrations of a system for treating fluid overload conditions of a patient using a fluid stimulation system according to some embodiments of the present disclosure.

FIG. 2 is a perspective view of an exemplary system consistent with the present disclosure, in which a patient is wearing one embodiment of a treatment device which is operably coupled to a console unit configured to provide heated air to the treatment device, which thereby provides a warm air environment at a controlled air volume flow rate around the body of the patient to shift fluids directly and non-invasively from an interstitial compartment of the patient to skin of the patient resulting in controlled fluid loss.

FIGS. 3A and 3B are block diagrams illustrating embodiments of the system of FIG. 1, which includes a wearable treatment device consistent with the present disclosure.

FIG. 4 is an image of an exemplary embodiment of a treatment device of the fluid stimulation system of the present invention.

FIGS. 5A and 5B are enlarged views of portions of the treatment device, including an air outlet port and air inlet port, respectively.

FIG. 6 is an enlarged view of a portion of the treatment device illustrating portions thereof in which sensors are placed for collecting associated data measurements during a treatment procedure.

FIG. 7 is an enlarged view of the air inlet port in which one or more sensors are placed.

FIG. 8 shows an exemplary embodiment of an infrared (IR) skin temperature sensor for use with the treatment device of the present invention.

FIG. 9 is an illustration of an inflatable assembly for use with the treatment device to provide spacing between a patient's body and an interior surface of the treatment device when placed upon the patient's body.

FIG. 10 is a plan view of the inflatable assembly of FIG. 9.

FIG. 11 is an image of a set of rigid spacer members for use with the treatment device to provide spacing between a patient's body and an interior surface of the treatment device.

FIGS. 12A, 12B, and 12C are images of interior portions of the treatment device, including another embodiment of spacer members (inflatable spacer members) provided on an interior back portion of the treatment device (FIGS. 12A, 12B) as well as a framework of tubing for distributing heated air throughout the treatment device (FIG. 12C).

FIG. 13A is an image of a fully assembled treatment device placed upon a patient, including enlarged views of sensors placed at various portions of the treatment device (FIGS. 13B and 13C).

FIGS. 14A and 14B are images showing an alternative embodiment of a treatment device in unassembled and assembled states.

FIG. 15 shows an assembled view of the wearable treatment device of FIG. 2.

FIG. 16 shows a patient wearing the wearable treatment device of FIG. 2.

FIG. 17 shows a patient wearing another embodiment of a wearable treatment device consistent with the present disclosure.

FIGS. 18A, 18B, and 18C show adjustable portions of the wearable treatment device of FIG. 6.

FIGS. 19A and 19B show cross-sectional views of the wearable treatment device, illustrating the multilayer design and chamber formed within for receiving air from the console unit and distributing such air throughout the wearable treatment device.

FIG. 20A shows a plurality of spacing members provided between the outer and inner layers of the wearable treatment device. FIGS. 20B and 20C are enlarged views of a spacing member.

FIG. 21 shows the wearable treatment device, partly in section, illustrating an interior surface of the device and placement of a spacer member placed at along the interior surface of the inner layer that corresponds to, and fits over, a posterior portion of a patient.

FIGS. 22A and 22B are rear and side views of a patient illustrating positioning of the rear spacer member when the wearable treatment device is worn by the patient.

FIG. 22C is a perspective view of the rear spacer member.

FIG. 22D shows plan and side views of the rear spacer member.

FIG. 23 is a plan view of another embodiment of a rear spacer member consistent with the present disclosure in the form of a cushioned member.

FIG. 24 is a perspective view of another embodiment of a rear spacer member consistent with the present disclosure in the form of an inflatable member.

FIG. 25 shows placement of an infrared (IR) skin temperature sensor within a corresponding portion of the rear spacer member for retaining the IR skin temperature sensor apparatus in place.

FIG. 26 is a perspective view of an exemplary embodiment of an IR skin temperature sensor apparatus to be used with the wearable treatment device.

FIG. 27 shows placement of an infrared (IR) skin temperature sensor within a corresponding portion of a rear spacer member of another embodiment of the wearable treatment device for retaining the IR skin temperature sensor apparatus in place, wherein skin temperature measurements can be communicated from the IR skin temperature sensor apparatus via a wired connection, such as a USB-C type connection or the like.

FIG. 28 shows one embodiment of an air inlet port provided on a portion (upper torso portion) of the wearable treatment device.

FIG. 29 shows placement of an inlet temperature/humidity sensor within a corresponding connector of the air inlet port for receiving and releasably retaining the inlet temperature/humidity sensor.

FIGS. 30 and 31 show different views of the connector portion on the air inlet port illustrating connection of the inlet temperature/humidity sensor within.

FIGS. 32A and 32B illustrate one embodiment of an air inlet port assembly comprising an inlet flange member and hose connector configured to engage and mechanically couple to the inlet flange member by way of a snap-fit connection means.

FIG. 33 shows an air outlet port provided on a portion (leg portion) of the wearable treatment device.

FIG. 34 shows an exemplary outlet temperature/humidity sensor placed within a flange portion of the outlet. FIGS. 35 and 36 show the flange portion of the outlet for fixating the outlet temperature/humidity sensor.

FIGS. 37A and 37B show exemplary air inflow and air outflow of a wearable treatment device consistent with the present disclosure in which various characteristics associated with air flowing into and air flowing out of the wearable treatment device may be measured via sensors provided in a shared connection assembly.

FIGS. 38A, 38B, and 38C show an exemplary connection assembly for coupling an external hose (from the console unit) to the wearable treatment device and for placing one or more inlet temperature/humidity sensors and one or more outlet temperature/humidity sensors into respective flow paths of inflowing air and outflowing air relative to the inlet and outlet ports.

FIGS. 39A and 39B illustrate a snap-fit connection between the connection assembly and an inlet connector portion of the wearable treatment device.

FIG. 40 shows a patient assembling the wearable treatment device.

FIG. 41 is an image showing an exemplary embodiment of a console unit consistent with the present invention.

FIGS. 42A and 42B are images showing front and rear views of an alternative embodiment of a console unit consistent with the present disclosure.

FIGS. 43A and 43B show front and rear perspective views, partly in section, of a console unit consistent with the present disclosure, including various components of the console unit.

FIG. 44 is a perspective view of an exemplary console unit used with the system of the present disclosure.

FIGS. 45A and 45B are side views of the console unit illustrating various internal components, including electrical and mechanical components.

FIG. 46 is a perspective view of the console unit illustrating portions of the airway assembly, including a filter duct and blower fan.

FIGS. 47A and 47B show a filter duct.

FIG. 48 is a perspective view of the console unit illustrating the heater cone component in greater detail.

FIG. 49 is side view of the heater cone coupled to a heater member.

FIG. 50 is an exploded perspective view of the heater cone of FIG. 48.

FIG. 51 is a perspective view of another embodiment of a heater cone component consistent with the present disclosure.

FIG. 52 is an exploded perspective view of the heater cone of FIG. 51.

FIG. 53A is a perspective view of the heater cone illustrating a flow sensor connection portion.

FIG. 53B shows placement of the flow sensor relative to the corresponding flow sensor connection of the heater cone.

FIG. 54A shows mounting of a thermocouple to a respective portion of the heater cone.

FIG. 54B shows an exemplary thermocouple.

FIG. 55 is a side view of the heater cone coupled to a heater.

FIG. 56 shows an air outlet assembly for coupling one end of a hose to the console unit.

FIG. 57 illustrates components of the air outlet assembly in greater detail.

FIG. 58 is an exploded perspective view illustrating components of the air outlet assembly in greater detail.

FIG. 59 shows three different embodiments of noise reducing designs for reducing overall noise of the console unit, particularly reducing noise caused during operation of the blower fan.

FIGS. 60A and 60B show an exploded and assembled view, respectively, of the components forming a chassis of the console unit.

FIG. 61 shows a partially exploded view illustrating placement of legs and wheels, as well as a hose holder, upon the assembled chassis.

FIG. 62 shows the various main electrical components provided in the console unit.

FIGS. 63, 64, and 65 illustrate various assembly processes for assembling electrical components to the chassis of the console unit.

FIG. 66 shows the various airway assembly components provided in the console unit.

FIG. 67 shows assembly of the airway components and placement within the chassis of the console unit.

FIGS. 68 and 69 show various assembly processes for assembly the top cover and associated components, including placement of the controller with touchscreen display, to the chassis of the console unit.

FIG. 70 shows assembly processes for coupling the front cover and associated components to the chassis of the console unit.

FIG. 71 shows assembly processes for coupling plastic panels to the chassis of the console unit.

FIG. 72 shows various acoustic panels to be placed relative to the chassis of the console unit and provide noise reduction.

FIG. 73 shows assembly processes for coupling exterior paneling to the chassis of the console unit and other portions thereof.

FIG. 74 shows a fully assembled console unit, including a hose holder for retaining a loose, unconnected portion of the hose (when the hose is uncoupled from a wearable treatment device and not in use).

FIG. 75 is a schematic block diagram of the console unit architecture.

FIG. 76 is a schematic block diagram one embodiment of console unit power supply scheme.

FIG. 77 is a circuit diagram representing one embodiment of an isolation scheme.

FIG. 78 is a schematic block diagram of the main board architecture.

FIGS. 79A, 79B, 79C, and 79D highlight communication, operation, safety, and wireless aspects of the schematic block diagram the main board architecture of FIG. 78.

FIG. 80 is a schematic block diagram of the main board power supply scheme.

FIG. 81 is a chart describing various safety features utilized by the console unit of the present disclosure.

FIG. 82 is a schematic block diagram illustrating one embodiment of a HW safety mechanism.

FIG. 83 is a circuit diagram representing one embodiment of a HW safety circuit.

FIG. 84 is a circuit diagram representing one embodiment of a temperature/humidity sensor architecture.

FIG. 85 is a circuit diagram representing one embodiment of an IR sensor architecture.

FIG. 86 is a block diagram illustrating communication of data between the wearable treatment device, sensors, console unit, and computing device, including an initial settings screen provided via a graphical user interface (GUI) of the computing device.

FIG. 87 is an exemplary screenshot of a user interface providing various information related to the fluid stimulation system, including details of a given procedure.

FIG. 88 illustrates an exemplary screenshot of a user interface providing various information related to the fluid stimulation system, including details of a given procedure.

FIG. 89 is a flow diagram of a system algorithm consistent with the present disclosure.

FIGS. 90, 91, 92, 93, and 94A-94B show clinical data from using systems and methods of the invention.

FIG. 95 is a block diagram illustrating communication of data from the various sensors to the console unit via a microcontroller (a STM32 Nucleo development board).

FIG. 96 is a block diagram illustrating one embodiment of controlling the temperature output of air (to be supplied to the wearable treatment device) via a proportional-integral-derivative (PID) controller.

FIGS. 97A and 97B are graphs illustrating temperature control via a conventional on/off operation and a via PID control consistent with the present disclosure, respectively.

### DETAILED DESCRIPTION

Expansion of extracellular volume is central to the pathophysiology of heart failure (HF) and other edematous disorders, resulting in a signs and symptoms (edema, dyspnea, orthopnea) commonly referred to as congestion. In ambulatory patients with HF, signs of congestion are strongly related to patient-assessed quality of life and future events. The main reason for hospitalization for worsening HF is related to fluid excess and symptoms of congestion.

Current treatments of congestion rely primarily on diuretics. As stated in a 2019 position statement of the Heart Failure Association of the European Society of Cardiology, “Other than ultrafiltration, the only pathway to get rid of sodium and water is through increased renal natriuresis and diuresis”. Unfortunately, even when fully adhering to these guidelines, adequate decongestion is not achieved in many patients. This leads to a gradual accumulation of fluid and sodium that ultimately leads to overt clinical symptoms. Failure to successfully manage congestion in the outpatient or hospital setting may be related, at least in part, to the limitations of diuretic therapy.

Humans have the capability of producing a large amount of sweat under certain physiological conditions. Eccrine glands are the major sweat glands located throughout the body. The eccrine glands open to the outside world through the sweat pores which produce clear, odorless sweat and are supplied directly from the interstitial compartment. Apocrine sweat glands are another type of sweat gland and are generally found in the armpit, ear, eyelids, and perineum. The secretory portion is larger than that of eccrine glands (making them larger overall). Rather than opening directly onto the surface of the skin, apocrine glands secrete sweat into the pilary canal of the hair follicle.

Sweat glands and sweat rate are influenced by local skin temperature. Sweat may be initiated at skin temperature as low as 33° C. and in a temperature range that varies between 33° C.-39° C. (the upper range value is set to avoid local skin burns). It is expected that a patient may excrete over 100 ml/h of sweat just from local elevation of skin temperature. This mode of increased sweat rate by elevating skin temperature may be beneficial with edematous patients as the sweat fluids flow from the interstitial compartment, being the source of the edema. In these patients, the clinical need (over 100 ml/h) can be achieved by exposing portions of the patient's body to elevated temperatures that will elevate the skin temperature to levels above 33° C. and below 40° C., and more preferably no greater than 39° C. The present invention recognizes the advantages of increased sweat rate for treating fluid overload. In particular, the fluid stimulation system of the present invention enhances fluid transfer through the skin, by increased sweat rate, by providing controlled stimulation of fluid transfer directly and non-invasively from an interstitial compartment of the patient through the skin of the patient.

The fluid stimulation system includes a wearable treatment device that can be placed over one or more portions or parts of a patient's body, including, but not limited to, one or more legs, one or more arms, one or more hands or feet, torso, lower back, and abdomen up to the level of the chest, for example. The treatment device is operably coupled to a console unit configured to provide heated air to the treatment device. In particular, the console may control air temperature, flow rate, humidity, and other additional parameters which assure safe and effective operation during use. Accordingly, upon being placed over the desired portions of the patient's body, the treatment device is able to create a controlled, homogenous warm air environment at a predetermined volume around the patient's body in order to create conditions that initiate sweat production. Throughout the treatment, fluids from the interstitial compartment are removed from the body by the eccrine and/or apocrine sweat glands, thereby decongesting fluid overloaded patients.

Systems of the invention include a home-use or an outpatient clinic device for chronic patients at a risk of developing fluid overload. The device is easily adjustable between treatment areas, is easy to operate and monitor, and is easy to clean and maintain. Embodiments of the device are portable, although the device can be static during treatment episodes.

The invention also allows for reading and monitoring of fluid removed, as well as the contents of the fluid. Treatment episodes can be local and isolated to body parts such as the leg, arm, abdomen, and back. Sweating can be regulated during treatment to allow for fluid flow from the skin of about 500 milliliters per day. The invention allows patients to ensure no skin or other heat injuries occur during the treatment and there are no excessive losses of electrolytes or salts that cannot be reabsorbed or digested back. Moreover, sweating is a process that most patients typically have experienced, and adverse effects to the skin are unlikely, even in severe heart failure patients.

Fluid transfer from the interstitial and intravascular compartment can be stimulated and enhanced via sweat, osmosis, other fluid transfer, or a combination thereof using local elevation in skin temperatures or increase in ambient osmotic pressure favoring fluid transfer from the interstitial compartment to outside the body.

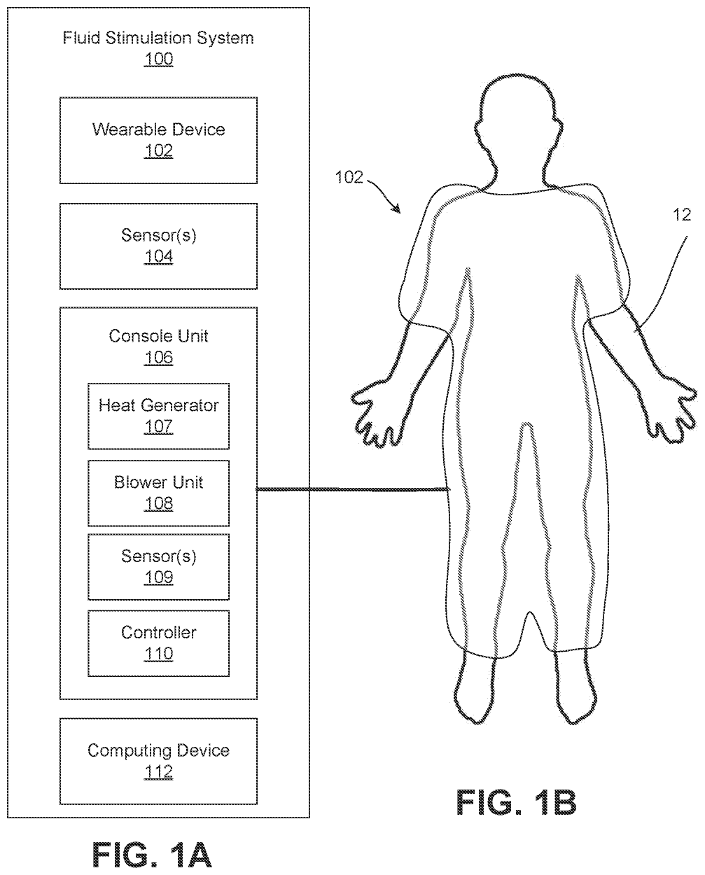

FIGS. 1A and 1B are diagrammatic illustrations of a fluid stimulation system 100 for treating fluid overload conditions of a patient 12. The system 100 generally includes a treatment device 102 and a console unit 106 to which the treatment device 102 is to be operably connected.

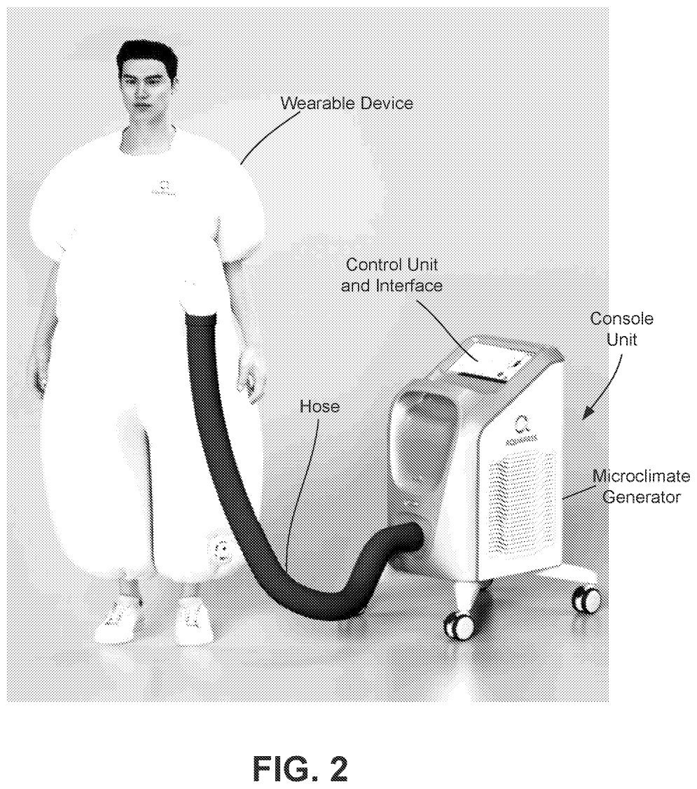

FIG. 2 is a perspective view of an exemplary system 100 consistent with the present disclosure, in which a patient is wearing a treatment device which is operably coupled to a console unit configured to provide heated air to the treatment device, which thereby provides a warm air environment at a controlled air volume flow rate around the body of the patient to shift fluids directly and non-invasively from an interstitial compartment of the patient to skin of the patient resulting in controlled fluid loss.

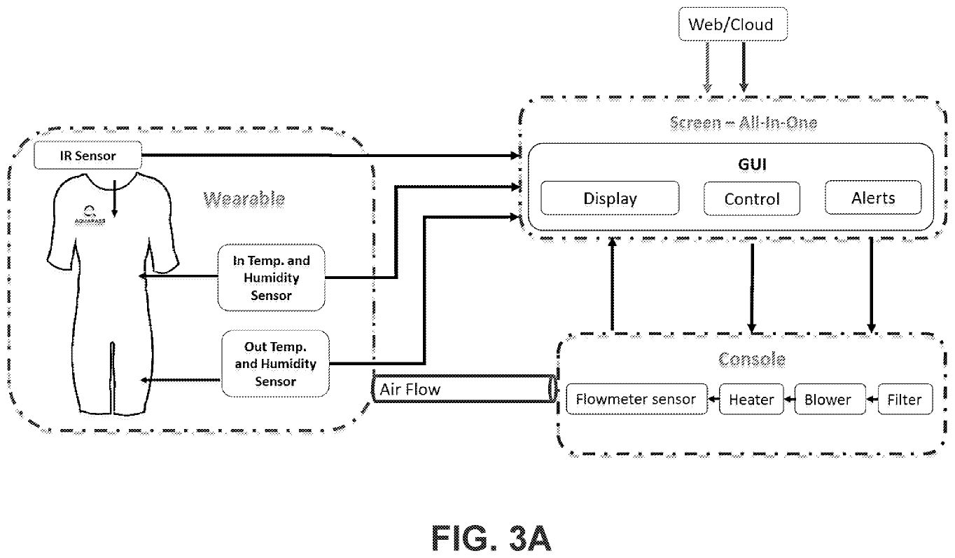

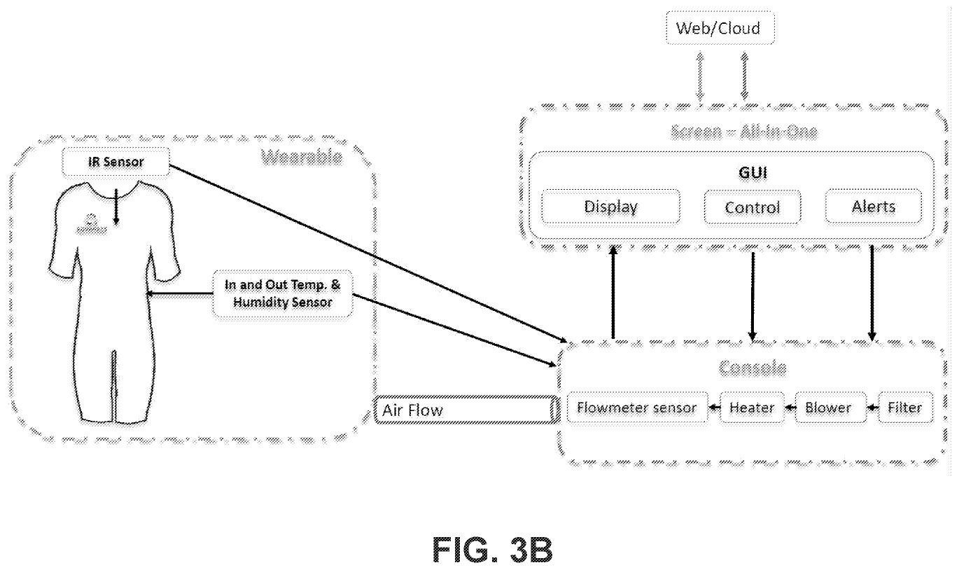

FIGS. 3A and 3B are block diagrams illustrating two embodiments of the system of FIG. 1, which includes the wearable treatment device illustrated in FIG. 2.

As illustrated, the treatment device 102 may be shaped and/or sized to be placed over one or more portions or parts of a patient's body, including, but not limited to, one or more legs, one or more arms, one or more hands or feet, torso, lower back, and abdomen up to the level of the chest, for example. In some embodiments, the treatment device 102 is sized to fit around a patient's abdomen, one or two legs, one or two arms, a back, or any combination thereof. In embodiments described herein, the treatment device 102 may cover a substantial portion of the patient's body, and may be in the is generally in the form of a full body suit, in which the legs, trunk or torso, chest, shoulders, and upper arms are covered, while the feet, lower arms and hands, neck, and head remain uncovered. In such an embodiment, the wearable treatment device may provide fluid loss at a rate of approximately more than 150 ml/hr during a procedure.

The treatment device 102 is operably coupled to a console unit 106 via a hose or other connection means through which heated air passed from the console unit 106 can flow into a corresponding inlet port on the treatment device 102. For example, a flexible PVC hose may be used. The hose may be releasably connected, at one end, to the inlet port of the device 102 and releasably connected, at an opposing end, to an outlet port of the console unit 106. For example, each end of the hose may include a snap connector mechanism configured to provide sufficient sealing and quick disconnection to and from the device 102 and console unit 106.

The console unit 106 is configured to provide heated air to the treatment device. In particular, the console unit 106 may include a heat generator 107 for heating air and a blower unit 108 for blowing the heated air out of the outlet port of the console unit 106 through the hose and into the treatment device 102.

The fluid stimulation system 100 further includes multiple sensors (104, 109) that can be used in controlling the output of heated air to the treatment device 102, as well as used in the continuous monitoring of the patient 12 during a given treatment session, including monitoring of patient vital signs. For example, temperature and humidity sensors 104 may be placed within an air flow path of the treatment device 102. In particular, the treatment device 102 may include an inlet port (e.g., for receiving heated air from the console unit 106 via a hose or other connection means) and an outlet port (e.g., for allowing heated air that has passed through portions of the treatment device 102 to exit the treatment device). The inlet and outlet ports may generally be coupled to a framework of channels or chambers of the treatment device 102 through which the heated air flows to provide the warm air environment within the interior of the treatment device (to which a patient is exposed). In some embodiments, each of the inlet and outlet ports may include respective temperature and relative humidity sensors 104 for monitoring temperature and relative humidity readings at the respect inlet and outlet ports.

Furthermore, the system 100 may include one or more skin temperature sensors 104 placed in contact with the skin of the patient for monitoring the skin temperature and used in verifying that the skin temperature does not, in any circumstance, elevate above a threshold value (i.e., less than 40° C., and more preferably no greater than 39° C.). The system 100 may also include one or more accelerometers 104 relative and in proximity to the patient's body for monitoring patient movement, specifically collecting acceleration data of the patient and used in determining a patient's pose (i.e., a supine or prone position, a sitting position, or a standing position). The console unit 106 may also include various sensors 109, including a temperature sensor, a flow meter (or flow sensor), and other sensors.

The sensors of the treatment device 102 and the console unit 106 provide data to a controller 110, which is configured to process and analyze such data for controlling and monitoring conditions of the treatment device. For example, during treatment, data measurements provided by the various sensors may be provided to the controller 110, which, in turn, is able to control the output of the console unit 106. For example, in the event that the skin temperature elevates above the threshold value (i.e., the maximum temperature at which the treatment can be safely performed, which could be as high as 39° C.), the controller 110 may terminate operation of the console unit 106, and thus prevents warm air from entering the treatment device 102. In some embodiments, the controller 110 processes temperature and relative humidity measurements collected via sensors 104 at the inlet and outlet ports of the treatment device 102 and, together with airflow measurements collected via a flow sensor 109 the console unit 106, is able to continuously calculate a sweat rate of the patient during the procedure.

Depending on the given treatment, including the specific parameters of the treatment (i.e., planned fluid removal amount and overall time for procedure), the controller 110 is able to control output from the console unit on-the-fly based on sweat weight calculations, including adjusting flow rate and/or inflow air temperature to the treatment device until a sufficient sweat rate is achieved.

The fluid stimulation system, most notably the console unit 106, including the heat generator 107, blower unit 108, and controller 110, may generally be controlled via a software-based application running either as a web-based app or running as a local app downloaded on a user's computing device 112. For example, a user (i.e., physician or other medical professional) may utilize a personal computing device 112 (e.g., laptop, tablet, smartphone, etc.) to access software associated with the fluid stimulation device, in which a user interface is provided allowing for the monitoring of a given procedure, as well as subsequent control of such procedure. The computing device 112 is configured to communicate and exchange data with the console unit and controller via a wired or wireless connection (i.e., Wi-Fi network, Bluetooth radio (including Bluetooth Low Energy), Near Field Communication (NFC), etc.). The computing device 112 may also be configured to communicate with other sensors not incorporated into either of the treatment device or console unit, including sensors for monitoring patient vital signs (i.e., heart rate, blood pressure, saturation, etc.).

Accordingly, the user interface provided on a user's computing device may provide real-time data during the procedure, including, but not limited to, information associated with a current treatment procedure (i.e., elapsed time since treatment began, sensor measurement data and related calculations of the environment provided by the treatment device, including air temperature and absolute humidity, skin temperature, current sweat rate, accumulative sweat measurement, and more). Furthermore, the user interface may provide a user with input controls for controlling the given treatment, including basic start and stop controls, controls for selecting as well as control over output from the console unit 106 (i.e., air flow rate and heat output).

Accordingly, upon being placed over the desired portions of the patient's body, the treatment device 102 is able to create a controlled, homogenous warm air environment at a predetermined volume around the patient's body in order to create conditions that initiate sweat production. Throughout the treatment, fluids from the interstitial compartment are removed from the body by the eccrine and/or apocrine sweat glands, thereby decongesting fluid overloaded patients.

It should be noted that the system of the present invention may further make use of steam during a procedure to aid in production of sweat from a patient undergoing the procedure. For example, in one embodiment, a steam generator may either be incorporated into the console unit or be provided as a standalone device. The steam generator may be configured to provide steam to the inflowing air. The steam may be introduced for the first few minutes of operation and is able to enhance a patient's sweat rate at an earlier stage of the procedure so as to avoid the time it may normally take for sweat to build up at the start of each procedure (i.e., forty-five minutes or longer). In particular, due to the low humidity in air, it may take time for the patient to start sweating when a procedure first begins. As such, the steam generator can be used to essentially humidify the incoming air for at least the first few minutes, which will result in a patient sweating sooner. Then, after a few minutes, the steam generator can be shut off or otherwise disconnected from the air supply, and sweating will continue during the procedure.

The steam generator can be in the form of a relatively small water dispersion unit, disposable, that can either be integrated into the console unit itself, or can be interconnected between the inlet port of the wearable treatment device and the consoler. In other words, the steam generator can be coupled to the console unit via a first hose and subsequently coupled to the inlet port of the wearable treatment device via a second hose and introduce a controlled amount of steam to the air flowing from the console to the wearable treatment device. The steam generator is configured to heat up water and provide steam to flow into the hose and the airflow pathway. For example, the steam generator may include a water reservoir and a heating element for heating water provided therein. Typically, it will be in the range of a 10cc to 50cc of water that will raise the relative humidity at the start of the procedure at the inlet port from around 20% to 60% for 5-10 minutes.

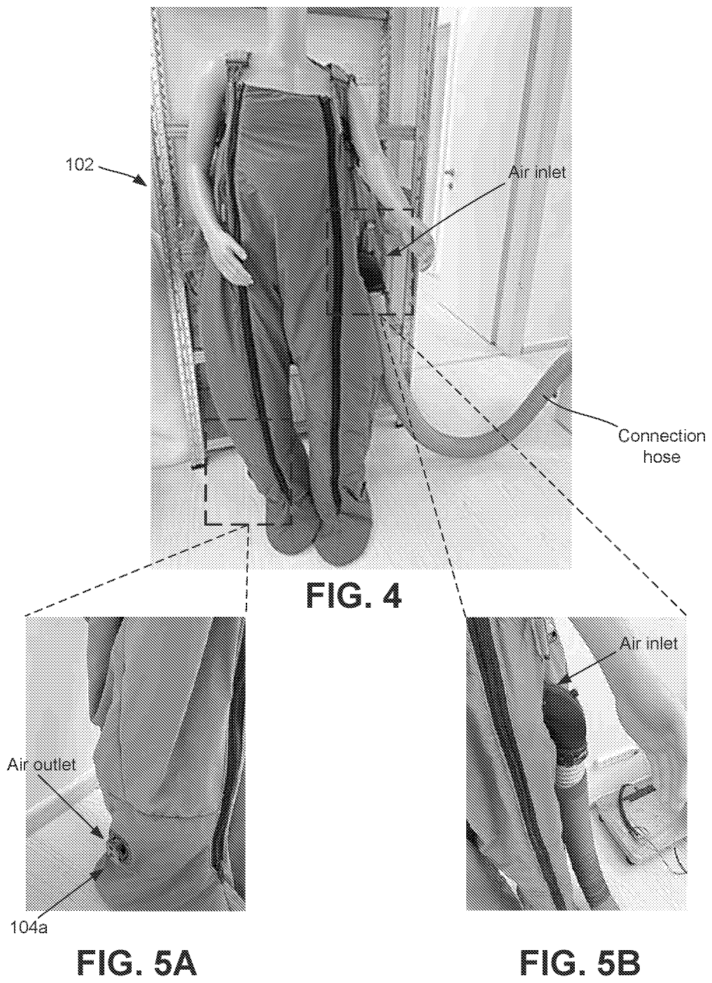

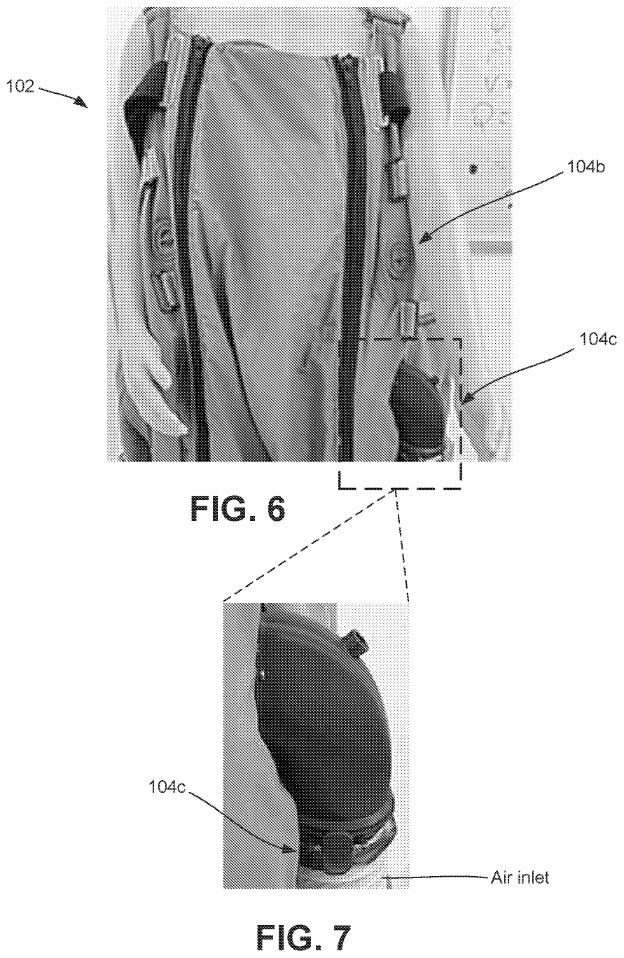

FIG. 4 is an image of an exemplary embodiment of a treatment device 102 of the fluid stimulation system 100. FIGS. 5A and 5B are enlarged views of portions of the treatment device 102 illustrating an air outlet port and air inlet port, respectively. FIG. 6 is an enlarged view of a portion of the treatment device 102 illustrating portions thereof in which sensors 104 are placed for collecting associated data measurements during a treatment procedure. FIG. 7 is an enlarged view of the air inlet port in which one or more sensors are placed.

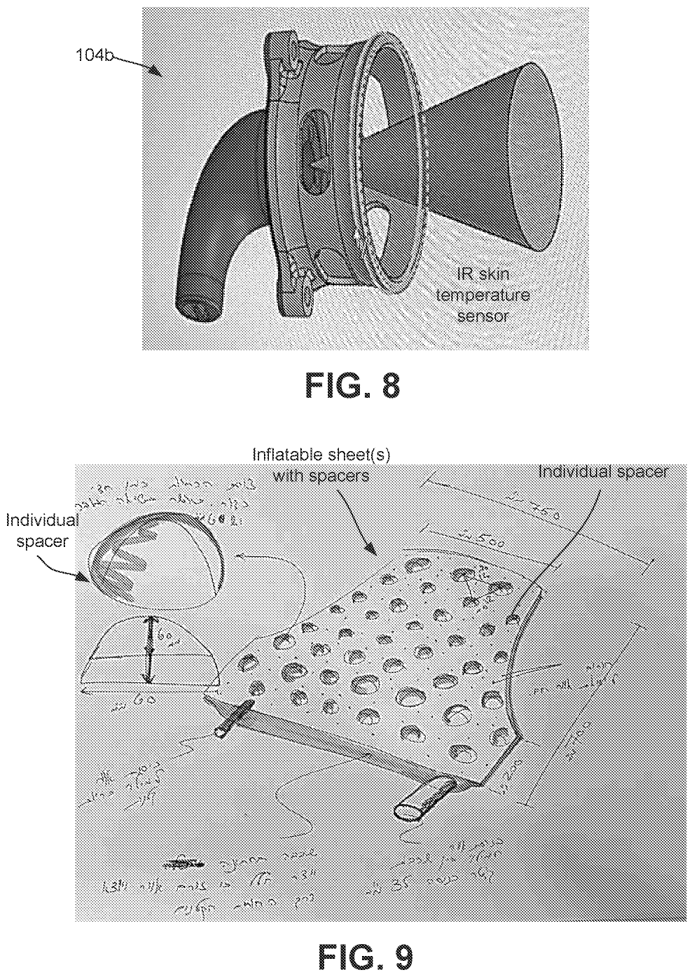

As shown, the treatment device 102 generally resembles clothing in the form of bib overalls (also known as coveralls), in which the legs and majority of the patient's trunk or torso are covered up to the chest level, while the arms, shoulders, neck, and head remain uncovered. Accordingly, the warm air environment provided by the treatment device 102 can cover at least the patient's legs and torso body parts. The treatment device 102 includes adjustable fasteners for enclosing the device over the patient, which may include hook and loop fasteners, zippers, straps, and the like. Accordingly, the device 102 can transform from an unassembled state to an assembled state. In the unassembled state, portions of the device 102 are separated from one another to allow a patient to place the device over their body. For example, the device may have zippers that fasten each leg portion of the device to one another and further fasten portions of the torso of the device to one another. Accordingly, once a patient places the device 102 on the appropriate body parts, a patient need only zip up the zippers and further tighten straps over their shoulders to obtain a tight, yet comfortable fit. The device 102 includes an air inlet to which the hose can be releasably coupled. The device 102 further includes an air outlet. As shown, one or more sensors 104a may be placed within the air outlet. The sensors 104a may include, for example, temperature and humidity sensors. Similarly, sensors 104c may be placed within the air inlet, wherein such sensors may include temperature and humidity sensors. The device 102 may further include a sensor for measuring the patient's skin temperature 104b, which may be located on a side of the treatment device 102 between the armpit and the air inlet. The temperature sensor 104b may include, for example, and infrared (IR) sensor, such as Infra Red Thermometer MLX90614, offered by Melexis). FIG. 8 shows an exemplary embodiment of an infrared (IR) skin temperature sensor for use with the treatment device of the present invention. As illustrated, IR skin temperature apparatus is positioned in the device wall so as to maintain a distance from the patient's skin, thereby enabling an IR beam to be focused to the sensor for data collection.

As previously described, the treatment device 102 is sized to fit around a body part of a patient, leaving clear volume of air between the body part and interior walls of the device 102. In particular, the invention allows for the skin to stay dry throughout the treatment. This is important because sweat that does not evaporate will slow down any further sweating and will also create an uncomfortable feeling for the patient. Anywhere there is contact between the patient and the device 102, the airflow is reduced and sweat can accumulate, resulting in less efficient sweating. Accumulation of sweat is prevented by keeping the body slightly away from the interior of the chamber. It is important to leave a clear volume of air, as it allows the device 102 to create a controlled, homogenous warm air environment around the patient's body

For example, in some embodiments, a disposable “net” is provided within the device 102, which assists in keeping the patient's body slightly away from the interior of the device 102. Disposable cloth may also be used to keep the body slightly away from the interior of the device 102, and would provide a benefit during cleaning of the device 102 between treatments.

Keeping the patient's body away from the interior of the device 102 further provides for uniform temperature of the air inside the device 102. Uniform temperature distribution inside the device 102 is important because an optimal temperature for sweating is between about 36° C. and about 39° C. Therefore, keeping the airflow in all parts of the body uniform allows for optimal sweat rates and avoidance of heat damage to the skin.

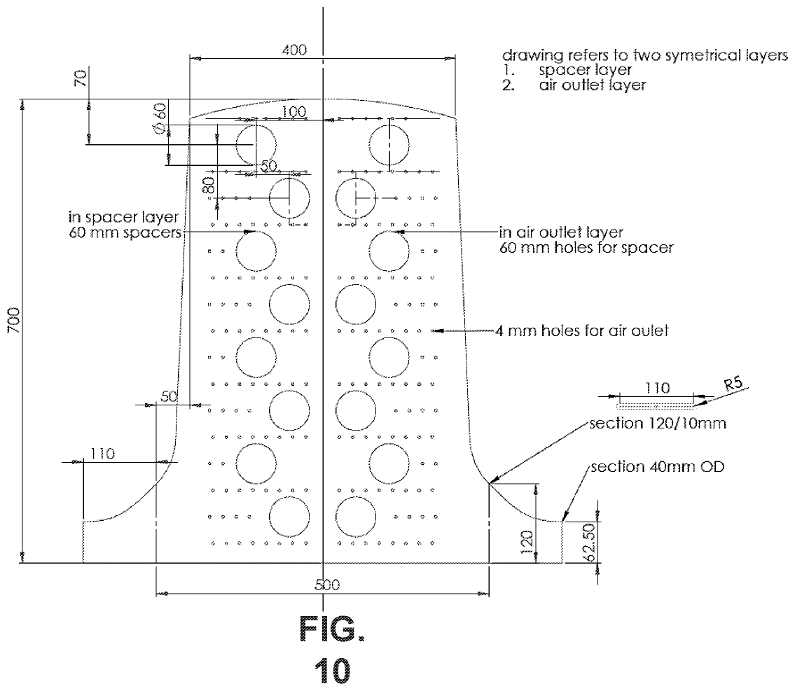

FIGS. 9-12C illustrate various embodiments for maintaining a slight separation between the interior of the device 102 and the patient's body.

For example, FIG. 9 is an illustration of an inflatable assembly for use with the treatment device 102. FIG. 10 is a plan view of the inflatable assembly of FIG. 9. As illustrated, the device 102 may include an inflatable assembly, which may be comprised of two layers of inflatable sheets, each having its own inflation tubes with no air connection between them. An upper layer sheet is intended to inflate spacers (shown as protrusions extending outwardly from the upper layer sheet) that assist in keeping the interior surface of the device 102 away from a portion of the patient's body (the back of the patient), thereby enabling free air flow and maximal evaporation area. It should be noted that those areas in which a patient's back makes contact with the interior of the device 102 wearable are minimized only to the extent that those areas are inflated. The inflation of the inflatable assembly can be provided via the console unit 106. Once the upper layer sheet is inflated, the air tubing that supplies air to the spacers is closed and kept closed until the end of the procedure. Accordingly, even in the event that the patient presses against the spacers, the spacers will maintain their structural integrity and keep the back away from the interior of the device 102, as the compressed air will not be able to escape, thus keep its strength. Once the upper layer is fully inflated and closed, the air from the console unit 106 may be diverted into a lower layer inflatable sheet. This second layer may include a plurality of apertures distributed across the surface (e.g., every few centimeters). Once the console unit 106 is operating, air enters the space between the upper and lower sheets and then the air is blown out towards the patient.

The inflatable assembly configuration of FIGS. 9 and 10 ensures that, on the one hand there is a constant volume of air between 35° C. and 46° C. around the patient and that this air volume is replaced at a rate of at least 0.5 cubic meters per minute to a maximum of 2 cubic meters per minute, and further maintaining the relative humidity lower than 60%, thereby ensuring full evaporation and optimal sweat rate.

The device 102 may also include a detachable module that is first worn by the patient, or it can also be assembled to the wearable and worn all at the same step. The detachable module is dual-purpose, in that it serves at least two needs. It is noted that, for the treatment to be efficient, the air around the patient must be homogenous with regard to temperature and humidity, there must be minimally contacting areas (as any given contact between the patient's body and interior of the device 102 may block sweat glands and inhibit the desired sweat response), and the patient must feel comfortable when they sit on a chair or lie down in bed. An apparatus that enables all these requirements has been developed. It generally consists of an inflatable vest that is worn around the lower and upper back, shoulders, chest and abdomen. The vest can be divided into two independent inflatable routes that are airtight also between them. The first inflation route inflates a mesh type air inflated unit that provide a back and front support for the patient and ensures that the interior of the device 102 is kept away from the patient, thus enabling free air flow. Once this support air path is inflated, the airline to this path is closed by a one way air valve and the air is then diverted to the air flow path. The air flow path follows the path of the support path and has multiple holes that enable the warm air to flow to the patient, thus initiating the sweating.

Other mechanisms for providing spacing between a patient's body and an interior surface of the treatment device are contemplated herein.

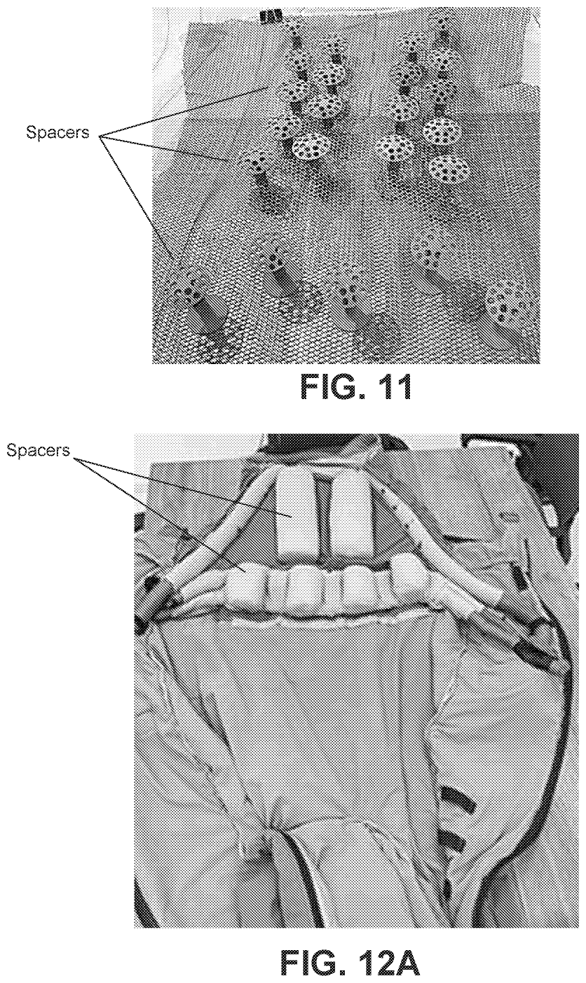

FIG. 11, for example, is an image of a set of rigid spacer members for use with the treatment device to provide spacing between a patient's body and an interior surface of the treatment device.

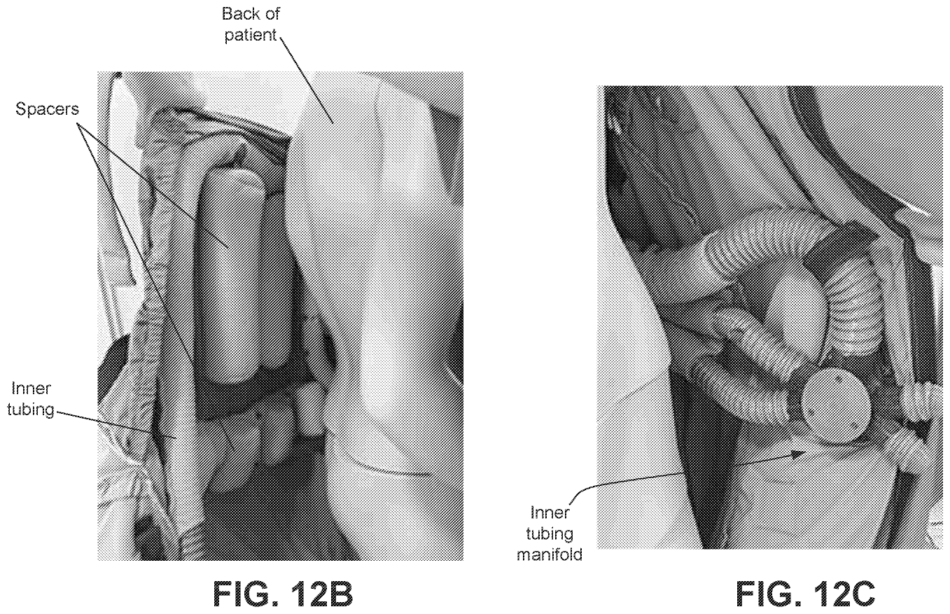

FIGS. 12A, 12B, and 12C are images of interior portions of the treatment device 102, including another embodiment of spacer members (inflatable spacer members) provided on an interior back portion of the treatment device (FIGS. 12A, 12B) as well as a framework of tubing for distributing heated air throughout the treatment device (FIG. 12C).

As previously noted, the fluid stimulation system 100 includes multiple sensors that can be used in controlling the output of heated air to the treatment device, as well as used in the continuous monitoring of the patient during a given treatment session, including monitoring of patient vital signs.

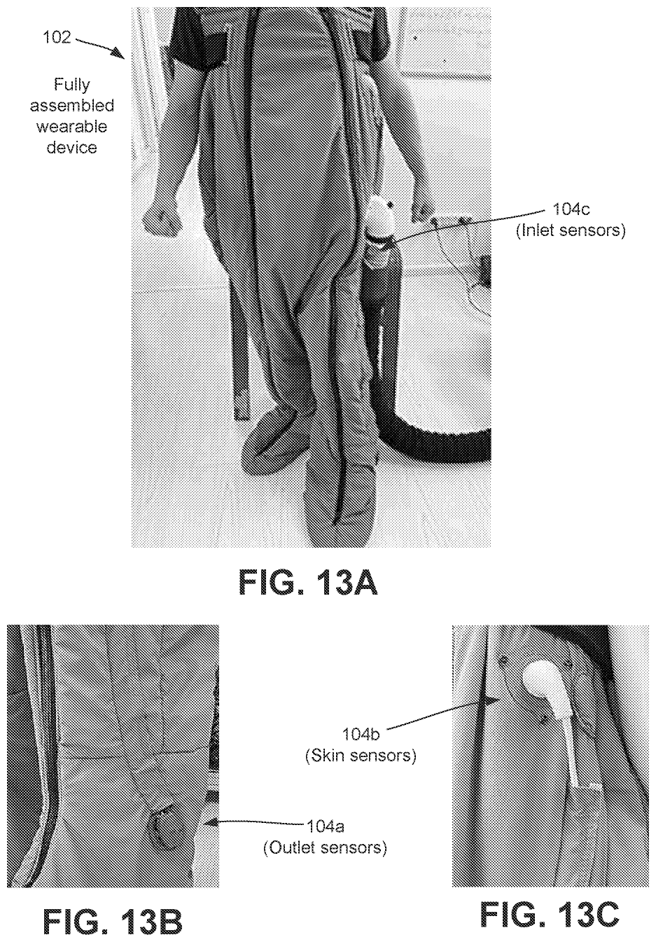

FIG. 13A is an image of a fully assembled treatment device 102 placed upon a patient, including enlarged views of sensors placed at various portions of the treatment device (FIGS. 13B and 13C). FIG. 13A shows sensors 104c to be placed proximate the inlet port. FIG. 13B shows sensors 104a to be placed proximate the outlet port. FIG. 13C shows a skin temperature sensor to be placed proximate a patient's armpit. The sensors 104a-104c can be coupled to the respective locations via a snap-fit type connection. Furthermore, sensor cables can be accommodated within portions of the treatment device 102, such as sleeves or the like.

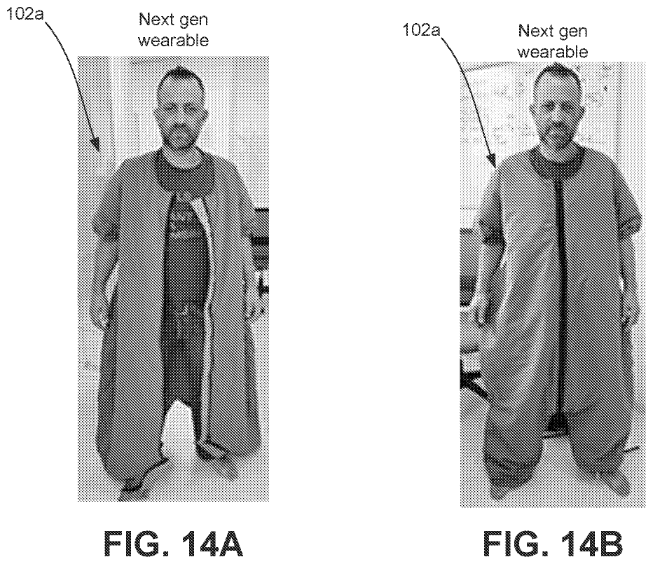

It should be noted that the treatment device 102 may have other shapes and sizes and it is not limited to the design illustrated herein. For example, FIGS. 14A and 14B are images showing an alternative embodiment of a treatment device 102a in unassembled and assembled states. As shown, the device 102a may further include sleeves that cover the shoulders and upper arm areas of a patient.

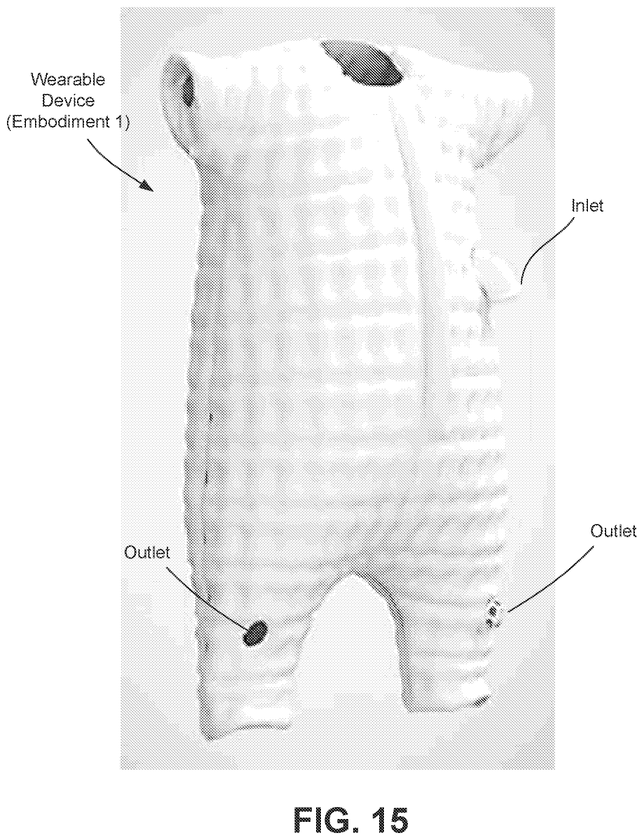

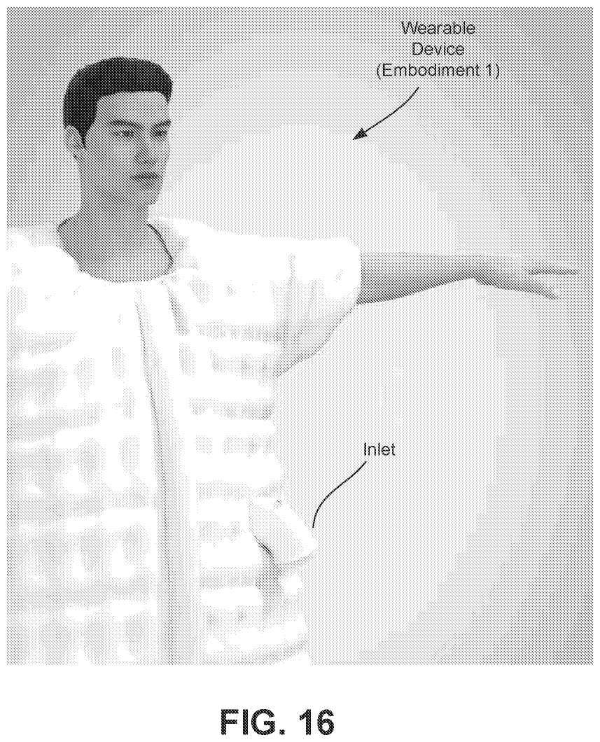

FIG. 15 shows an assembled view of the wearable treatment device of FIG. 2. FIG. 16 shows a patient wearing the wearable treatment device of FIG. 2.

As shown, the wearable treatment device is generally in the form of a full body suit, in which the legs, trunk or torso, chest, shoulders, and upper arms are covered, while the feet, lower arms and hands, neck, and head remain uncovered.

The wearable treatment device includes adjustable fasteners for enclosing the device over the patient, which may include hook and loop fasteners, zippers, straps, and the like.

Accordingly, the device 102 can transform from an unassembled state to an assembled state. In the unassembled state, portions of the device 102 are separated from one another to allow a patient to place the device over their body. For example, the device may have zippers that fasten each leg portion of the device to one another and further fasten portions of the torso of the device to one another. Accordingly, once a patient places the device 102 on the appropriate body parts, a patient need only zip up the zippers and further tighten straps over their shoulders to obtain a tight, yet comfortable fit

The device 102 includes an air inlet to which the hose can be releasably coupled. The device 102 further includes one or more air outlets. In the illustrated embodiment, the device includes two outlets (one outlet positioned on each leg portion of the device).

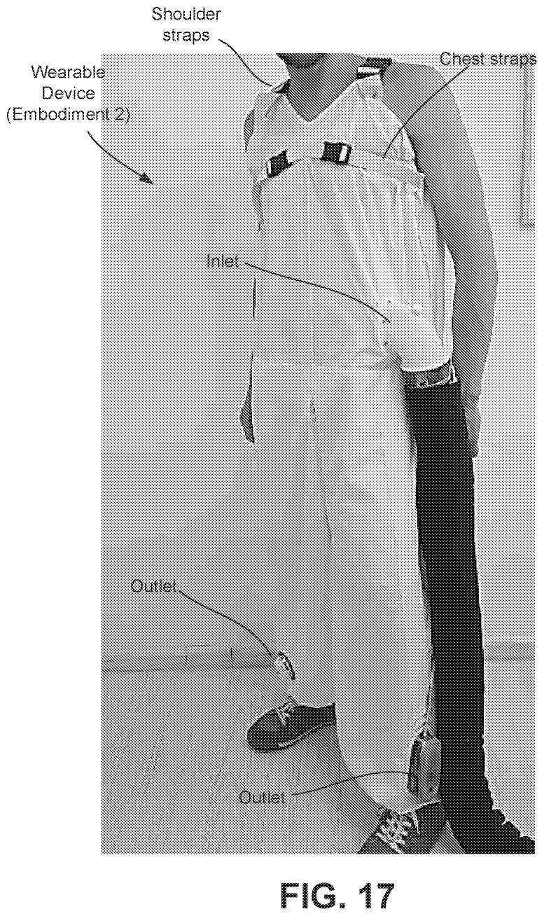

FIG. 17 shows a patient wearing another embodiment of a wearable treatment device consistent with the present disclosure. As illustrated, the treatment device resembles clothing in the form of bib overalls (also known as coveralls), in which the legs and majority of the patient's trunk or torso are covered up to the chest level, while the arms, shoulders, neck, and head remain uncovered. Accordingly, the warm air environment provided by the treatment device can cover at least the patient's legs and torso body parts.

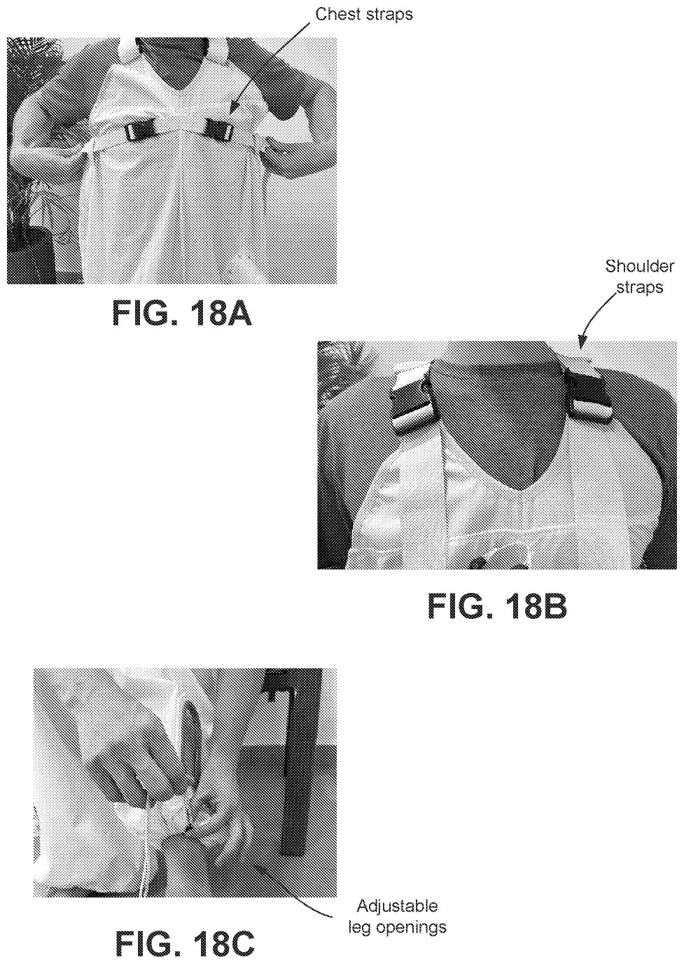

As shown, the treatment device includes adjustable fasteners for enclosing the device over the patient, which may include hook and loop fasteners, zippers, straps, and the like. For example, in the illustrated embodiment, the device includes at least adjust chest straps, shoulder straps, and leg openings to allow for the device to accommodate various shapes and sizes of patients, and further allow adjustability of the device to provide a tight fit upon the patient's body and prevent air flow leakage. For example, FIGS. 18A, 18B, and 18C show adjustable portions of the wearable treatment device of FIG. 17. Accordingly, the device can transform from an unassembled state to an assembled state.

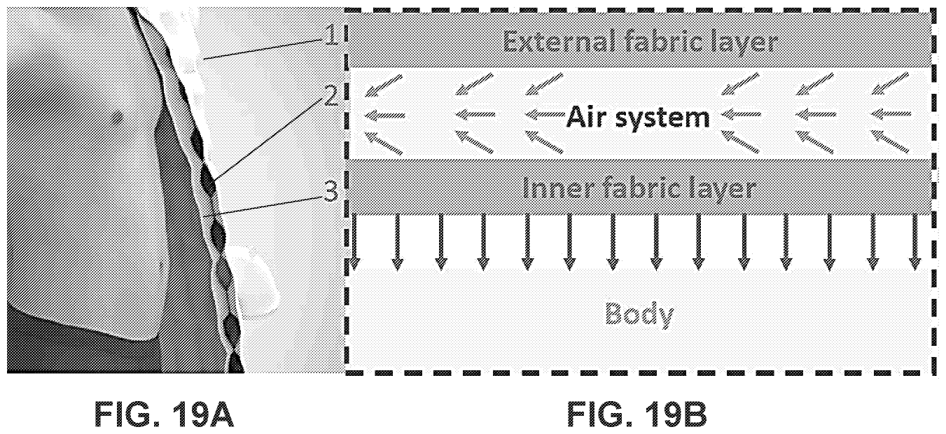

As previously described, the device 102 is designed to maintain a comfortable controlled environment around the patient's body during a given treatment. The device 102 may be made of multiple layers of fabrics, such as two or more layers. For example, FIGS. 19A and 19B show cross-sectional views of the wearable treatment device 102, illustrating the multilayer design. As shown, the device includes an outer layer of material and an inner layer of material cooperatively forming a chamber therebetween for receiving air from the console unit and distributing such air throughout the device. The outer and inner layers are joined to another via a plurality of separate fixation points. Any technique for joining the material together can be used. In a preferred embodiment, the outer and inner layers are joined to another via fabric welding. The pattern of fixation between the outer and inner layers may generally resemble a quilt-like configuration. The inner layer comprises a plurality of apertures allowing air to flow from the chamber through the inner layer and provide the warm air environment at a controlled air volume flow rate around a body part of a patient wearing the wearable device.

The interior surface of the inner layer, which may contact the patient's body, is designed to provide a comfortable texture and feel. The combination of multiple layers provides the wearable characteristics of water repelling, waterproof, moisture-controlling, wind repelling, and breathability. The multiple layer design, and combination of materials, provides optimal isolation against liquids and maintains the desired inner temperature and humidity levels required for treatment, as well as breathability. The outer and inner layers may be made of polyester thermoplastic polyurethane (TPU) materials, for example.

In some embodiments, the wearable treatment device may further include inner spacers provided between the outer and inner layers so as to prevent collapsing of the chamber in the event that the outer and inner layers are forced closed upon one another. For example, in the event that a patient is lying down (generally in a supine position), the force of the patient's body (posterior or back portion) upon the inner layer may then result in the inner layer contacting the outer layer and essentially collapsing a portion of the chamber formed therebetween, thereby blocking airflow through that portion of the chamber.

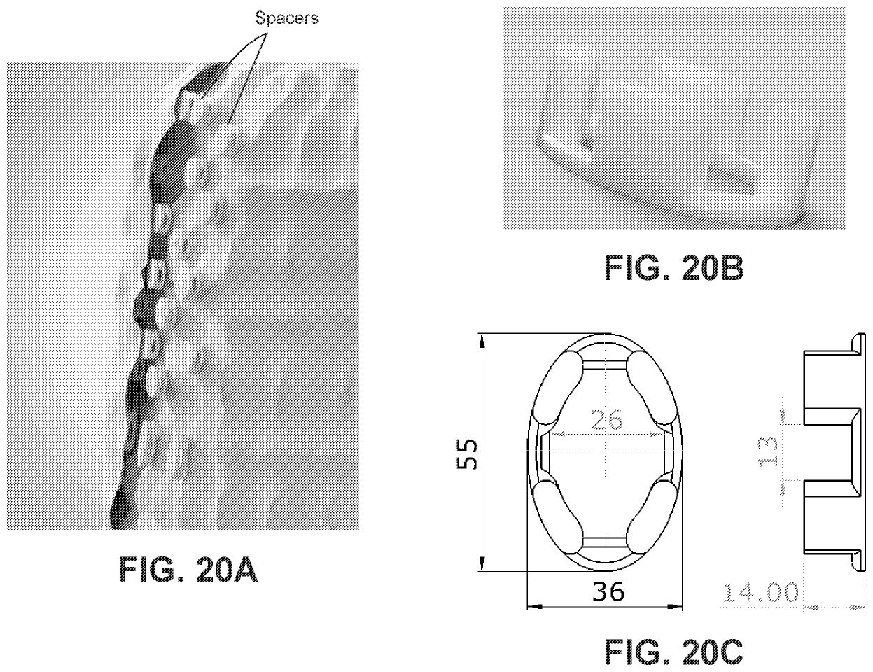

FIG. 20A shows a plurality of spacing members provided between the outer and inner layers of the wearable treatment device. FIGS. 20B and 20C are enlarged views of a spacing member. As shown, a plurality of spacing members may be provided between the outer and inner layers at a rear portion of the wearable device corresponding to, and fitting over, a posterior portion of the patient. Each of the plurality of spacing members is configured to maintain air flow within the chamber at the rear portion of the wearable device when the patient is in either of a supine position or seated position when wearing the wearable device. The spacers may generally be made of a TPU material.

The treatment device 102 is sized to fit around a patient's body, while also leaving a clear volume of air between the patient's body and an interior surface of the interior layer of the device 102. In particular, the invention allows for the skin to stay dry throughout the treatment. This is important because sweat that does not evaporate will slow down any further sweating and will also create an uncomfortable feeling for the patient. Anywhere there is contact between the patient and the device 102, the airflow is reduced and sweat can accumulate, resulting in less efficient sweating. Accumulation of sweat is prevented by keeping the body slightly away from the interior of the chamber. It is important to leave a clear volume of air, as it allows the device 102 to create a controlled, homogenous warm air environment around the patient's body.

Furthermore, maintaining a sufficient distance between the patient's body and the interior surface of the interior layer of the device provides for uniform temperature of the air within the environment created surrounding the patient's body. Uniform temperature distribution inside the device 102 is important because an optimal temperature for sweating is between about 36° C. and about 39° C. Therefore, keeping the airflow in all parts of the body uniform allows for optimal sweat rates and avoidance of heat damage to the skin.

Accordingly, in some embodiments, a spacer member may be positioned along an interior surface of the inner layer of the device so as to provide spacing between a patient's body and an interior surface of the device.

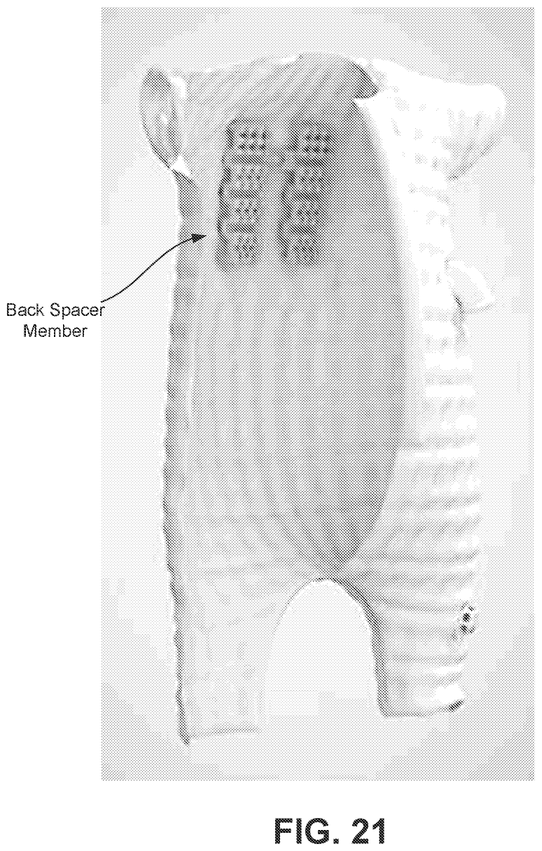

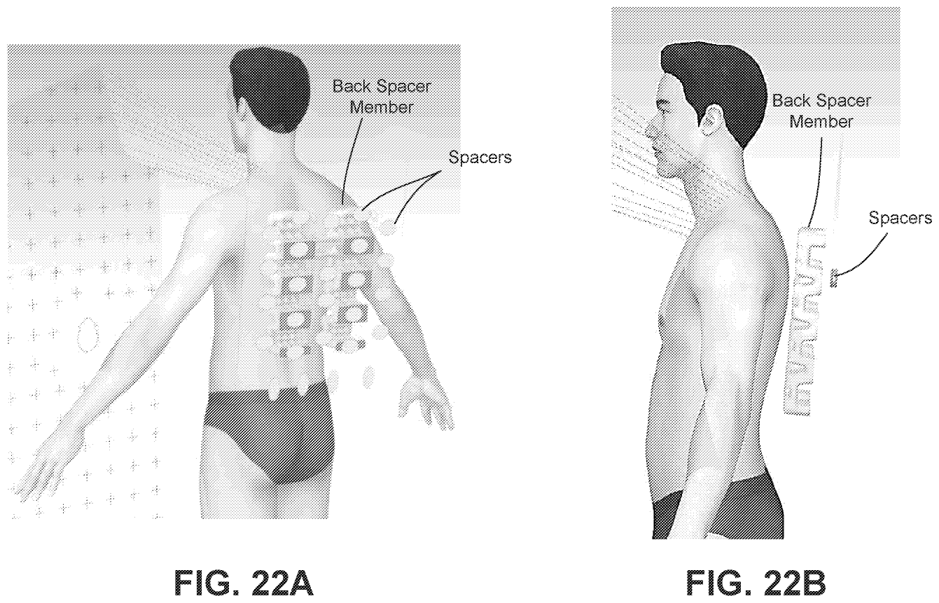

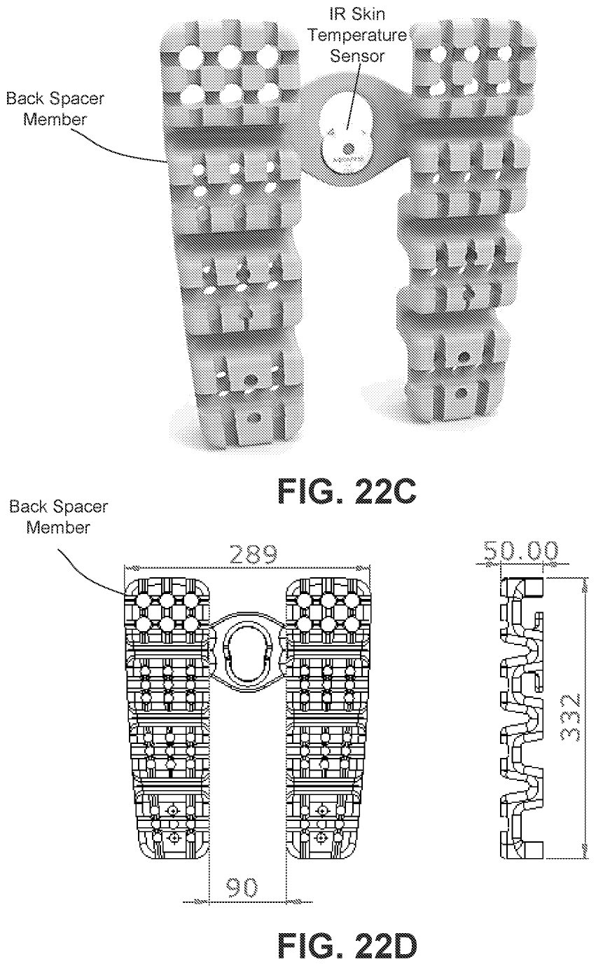

FIG. 21 shows one embodiment of the wearable treatment device, partly in section, illustrating an interior surface of the device and placement of a spacer member placed at along the interior surface of the inner layer that corresponds to, and fits over, a posterior portion of a patient. FIGS. 22A and 22B are rear and side views of a patient illustrating positioning of the rear spacer member when the wearable treatment device is worn by the patient. FIG. 22C is a perspective view of the rear spacer member. FIG. 22D shows plan and side views of the rear spacer member. The rear spacer member is configured to contact a posterior portion of the patient and maintain separation of the interior surface of the inner layer from the posterior portion of the patient when the patient is in either a supine position or seated position. As shown, the rear spacer member comprises a plurality of channels and/or apertures configured to maintain adequate air flow within the warm air environment surrounding the patient when the patient is in either a supine position or seated position and to further prevent blockage of sweat glands. The rear spacer member may be made from a TPU and/or ethylene-vinyl acetate (EVA) material. Accordingly, in addition to providing spacing, the rear spacer member may further provide comfort for the patient (i.e., improved support upon their back), especially when in a seat position.

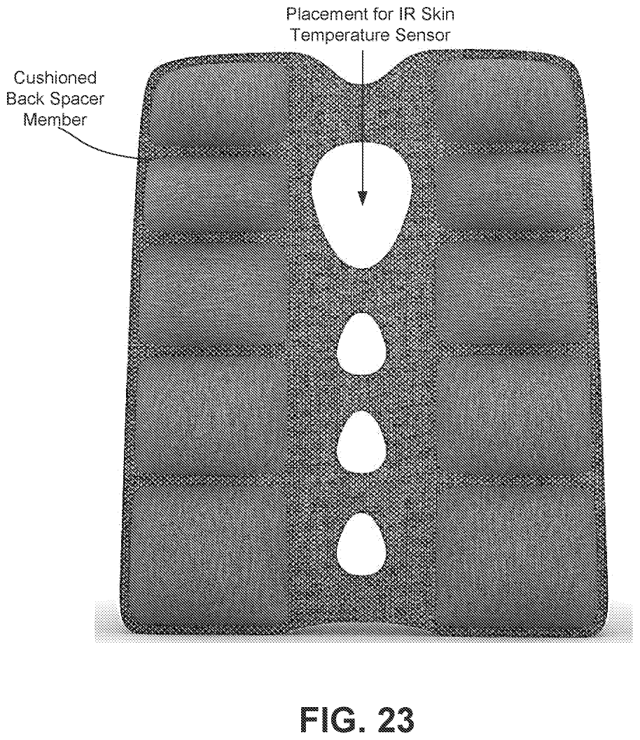

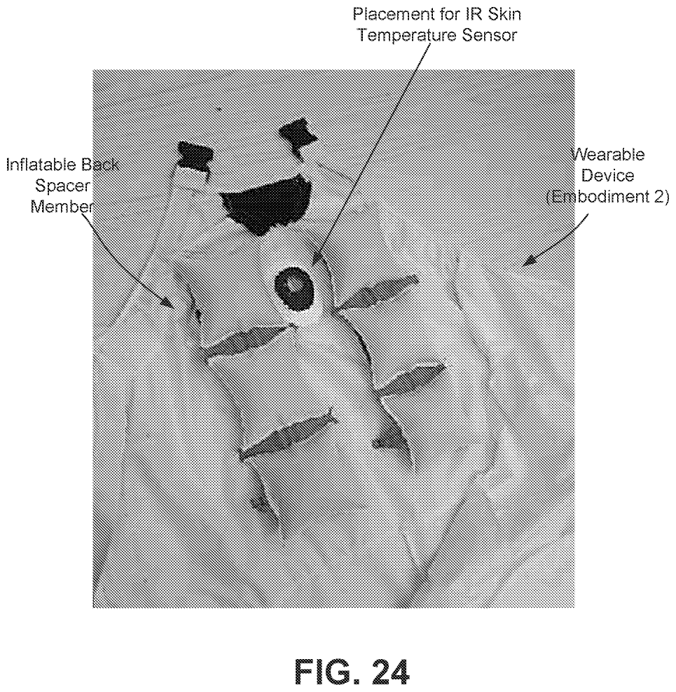

It should be noted that other embodiments of rear spacer members are contemplated herein. For example, FIG. 23 is a plan view of another embodiment of a rear spacer member consistent with the present disclosure in the form of a cushioned member. FIG. 24 is a perspective view of another embodiment of a rear spacer member consistent with the present disclosure, generally in the form of an inflatable member. Both the cushioned and inflatable rear spacer members are configured to provide similar benefits as the rear spacer member of FIGS. 21 and 22A-22D. More specifically, the rear spacer members configured to prevent the blockage of airflow through the warm environment created by the device 102, particularly when a patient is seated and/or lying in supine position. In particular, the rear spacer members are configured to contact a posterior portion of the patient and maintain separation of the interior surface of the inner layer of the device from the posterior portion (i.e., a back portion) of the patient when the patient is in either a supine position or seated position. Furthermore, the rear spacer members enable fixation of one or more sensors thereto for capturing measurement during a procedure, including skin temperature measurements.

As previously described, the wearable treatment device comprises multiple sensors for monitoring various parameters during a procedure.

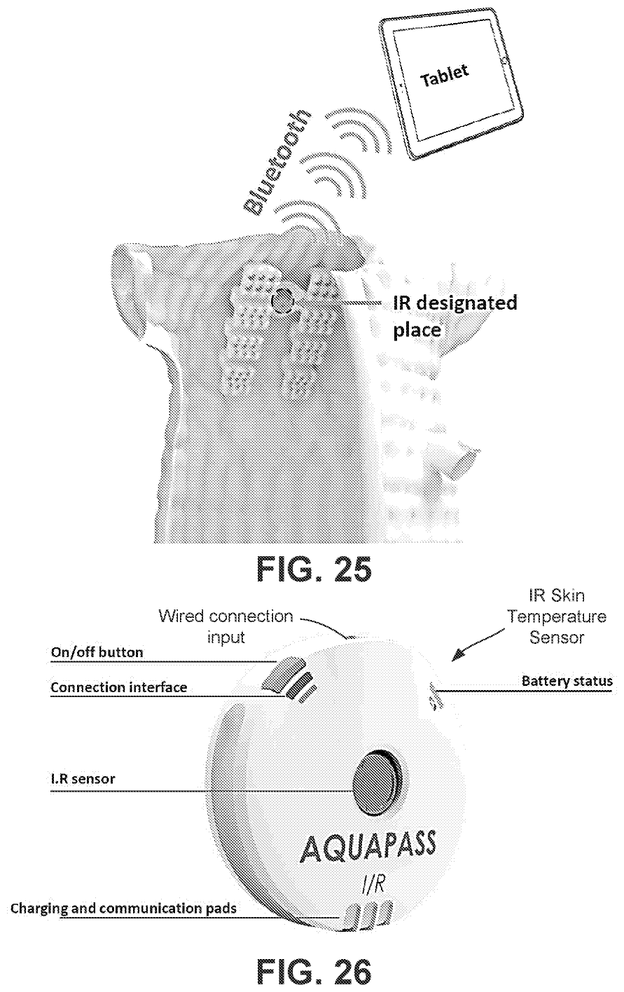

FIG. 25 shows placement of an infrared (IR) skin temperature sensor apparatus within a corresponding portion of the rear spacer member for retaining the IR skin temperature sensor apparatus in place. FIG. 26 is a perspective view of an exemplary embodiment of an IR skin temperature sensor apparatus to be used with the wearable treatment device. As shown, the rear spacer member may generally include a portion configured to receive and retain the IR skin temperature sensor in place and at a desired distance from a patient's skin. The IR skin temperature sensor includes, for example, an infrared (IR) sensor, such as Infra Red Thermometer MLX90614 (offered by Melexis). As illustrated, the IR skin temperature sensor apparatus is positioned in the rear spacer member so as to maintain a distance from the patient's skin, thereby enabling an IR beam to be focused to the sensor for data collection. The IR skin temperature sensor apparatus may be configured to wirelessly communicate data with the console unit, for example. The IR skin temperature sensor apparatus may also include a rechargeable battery.

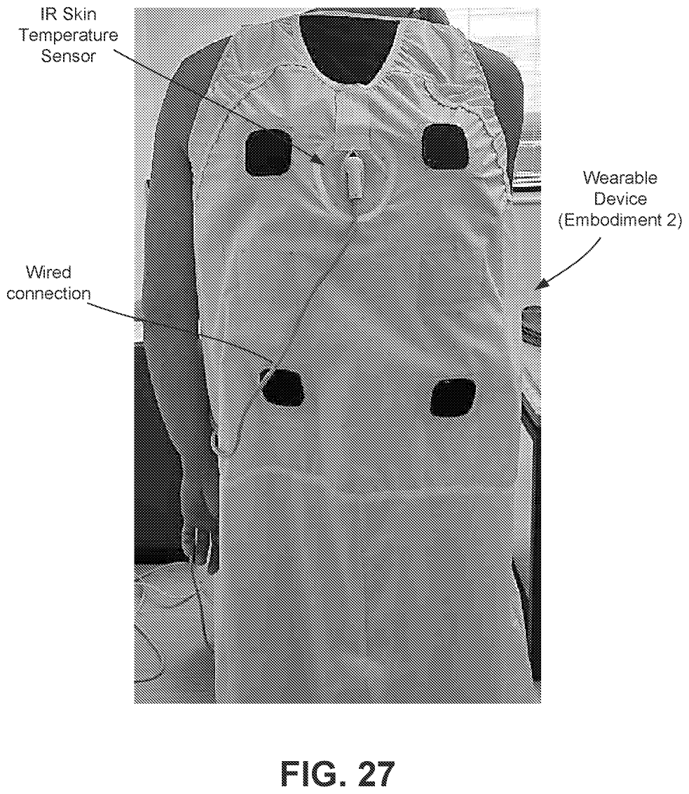

In some embodiments, the apparatus may also include connection ports for communicating data with the console unit via a wired connection. For example, FIG. 27 shows placement of an IR skin temperature sensor within a corresponding portion of a rear spacer member provided in one embodiment of the wearable treatment device, wherein skin temperature measurements can be communicated from the IR skin temperature sensor apparatus via a wired connection, such as a USB-C type connection or the like.

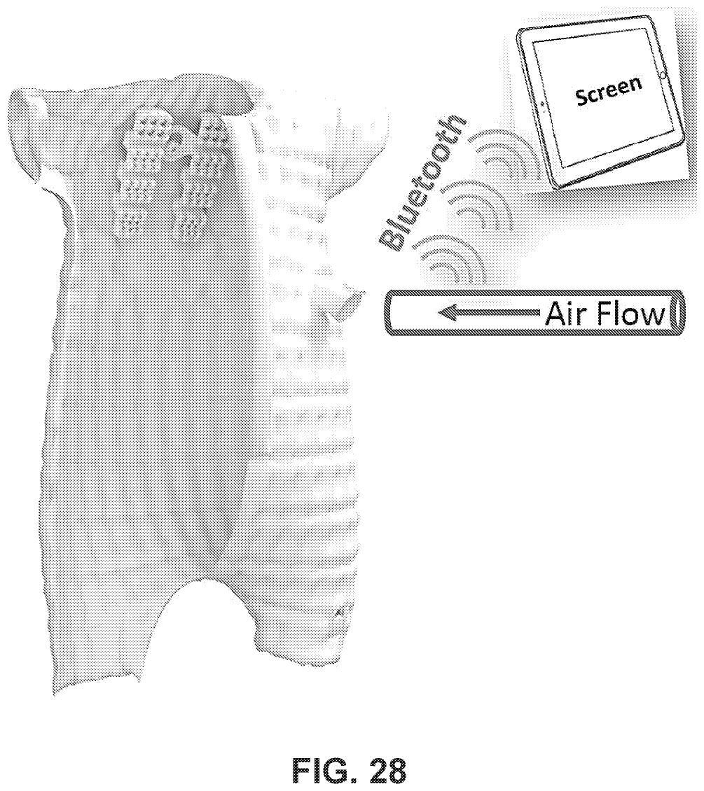

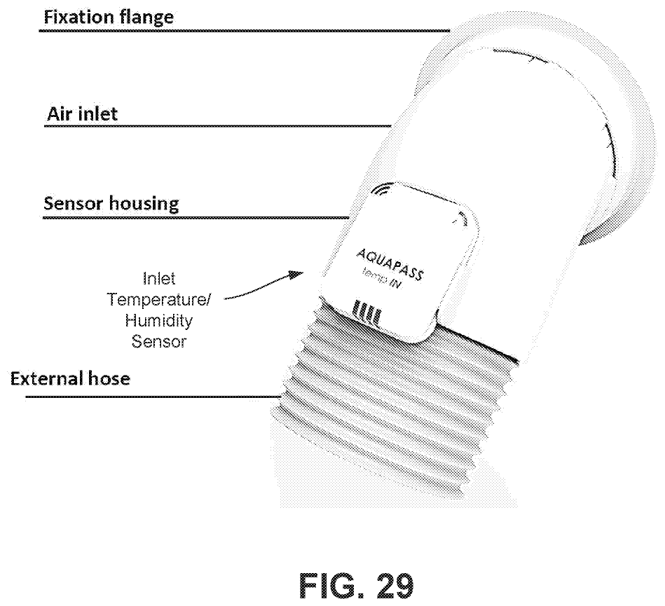

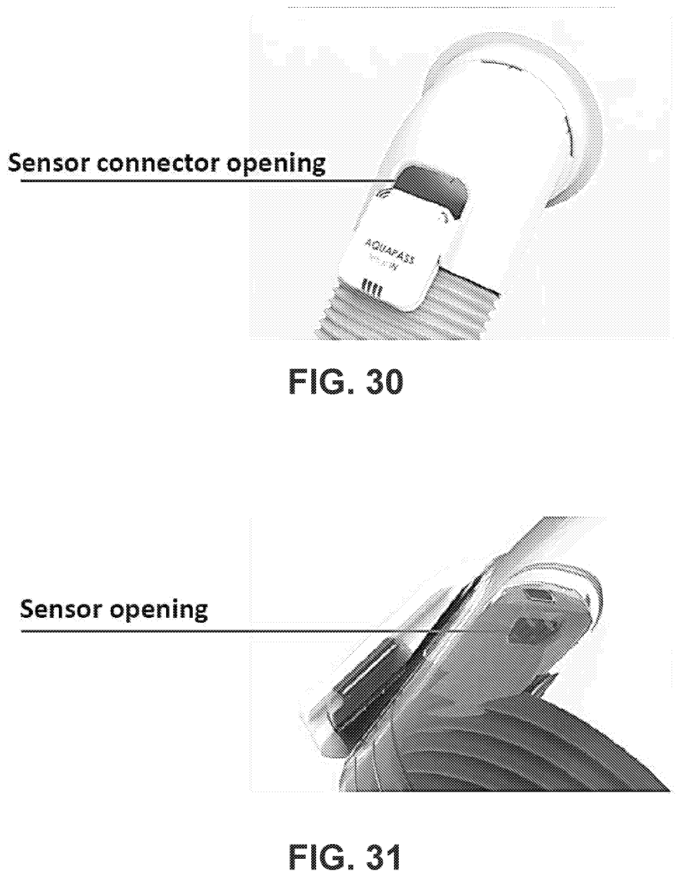

FIG. 28 shows an air inlet port provided on a portion (upper torso portion) of the wearable treatment device. FIG. 29 shows placement of an inlet temperature/humidity sensor within a corresponding connector of the air inlet port for receiving and releasably retaining the inlet temperature/humidity sensor. FIGS. 30 and 31 show different views of the connector portion on the air inlet port illustrating connection of the inlet temperature/humidity sensor within The inlet temperature/humidity sensor may include, for example, a digital humidity and temperature sensor SHT45 (offered by Sensirion).

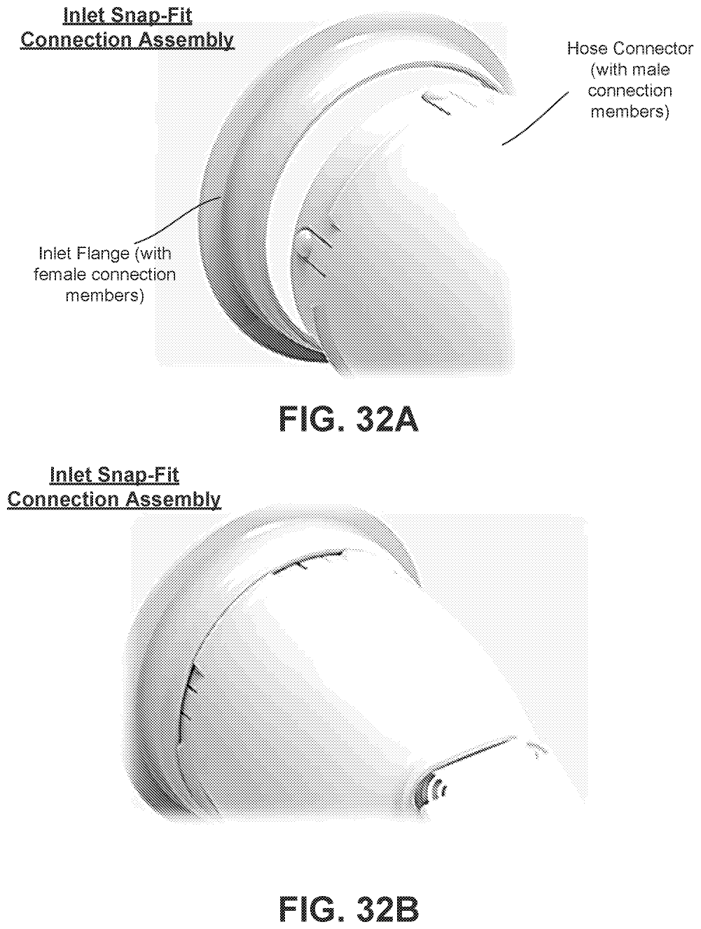

FIGS. 32A and 32B illustrate one embodiment of an air inlet port assembly comprising an inlet flange member and hose connector configured to engage and mechanically couple to the inlet flange member by way of a snap-fit connection means.

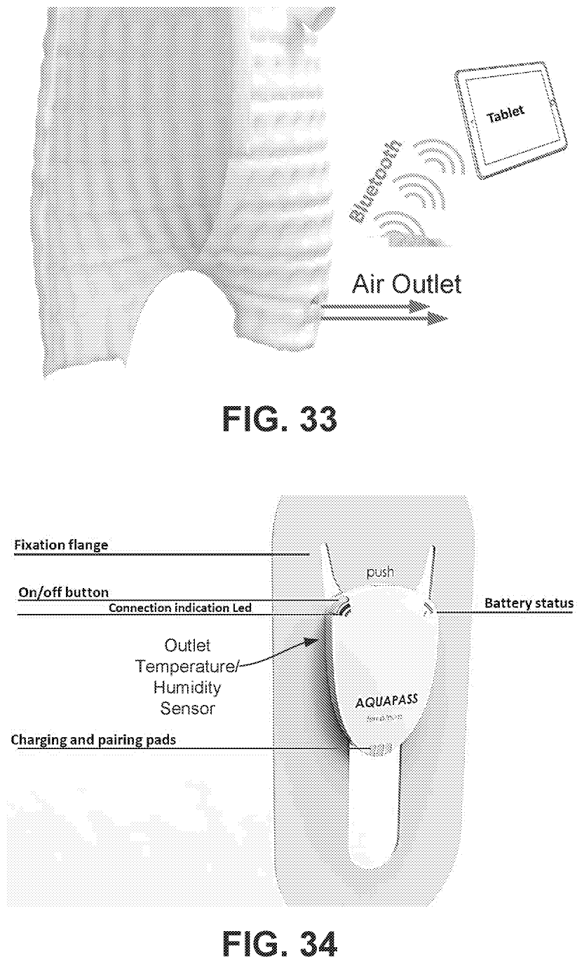

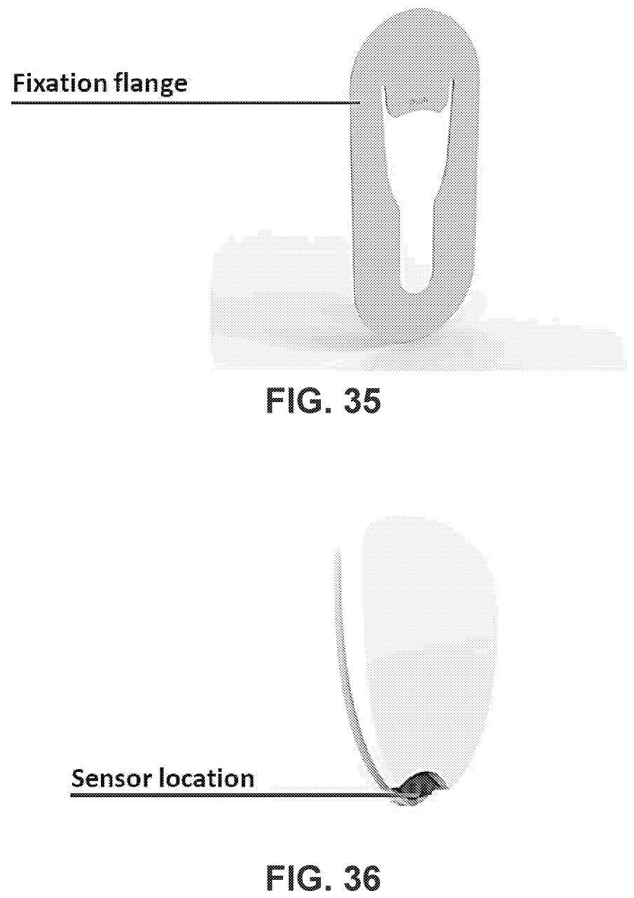

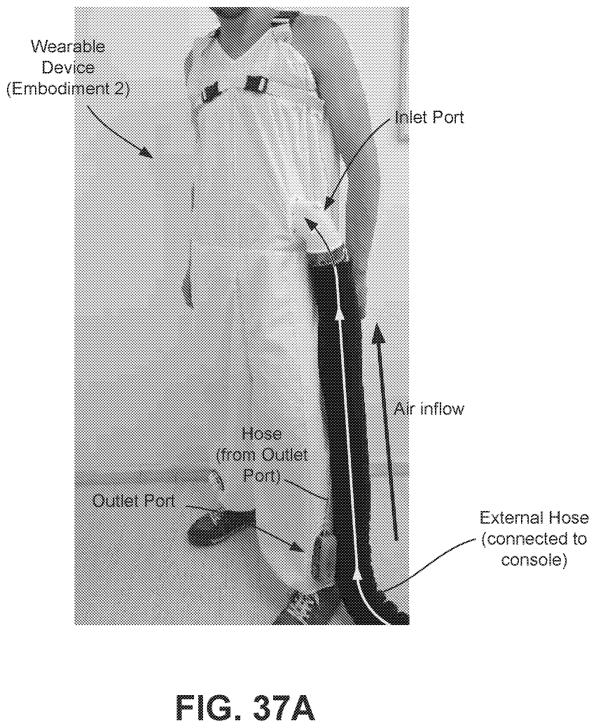

FIG. 33 shows an air outlet port provided on a portion (leg portion) of the wearable treatment device. FIG. 34 shows an exemplary outlet temperature/humidity sensor placed within a flange portion of the outlet. FIGS. 35 and 36 show the flange portion of the outlet for fixating the outlet temperature/humidity sensor.

The various sensors of the wearable treatment device collect and transmit associated data to the console unit (specifically a controller of the console unit), which is configured to process and analyze such data for controlling and monitoring conditions of the wearable treatment device. For example, during treatment, data measurements provided by the various sensors may be provided to the controller, which, in turn, is able to control the output of the console unit.

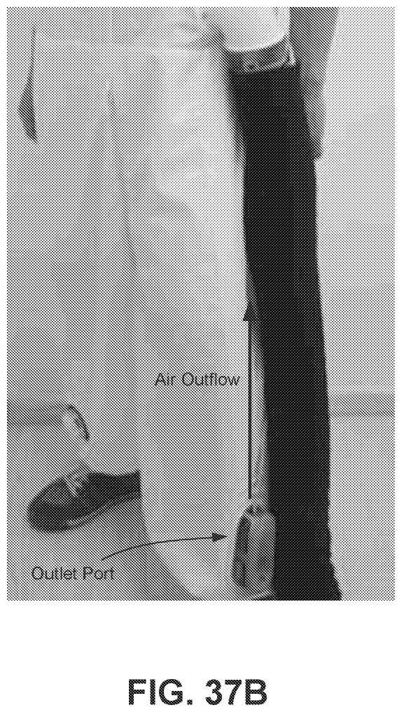

It should be noted that, in some embodiments, inlet and outlet sensors may be housed within a common unit. For example, FIGS. 37A and 37B show exemplary air inflow and air outflow of another embodiment of a wearable treatment device consistent with the present disclosure. As shown, while the wearable device comprises at least one inlet and at least one outlet, various characteristics associated with air flowing into and air flowing out of the wearable treatment device may be measured via sensors provided in a shared connection assembly.

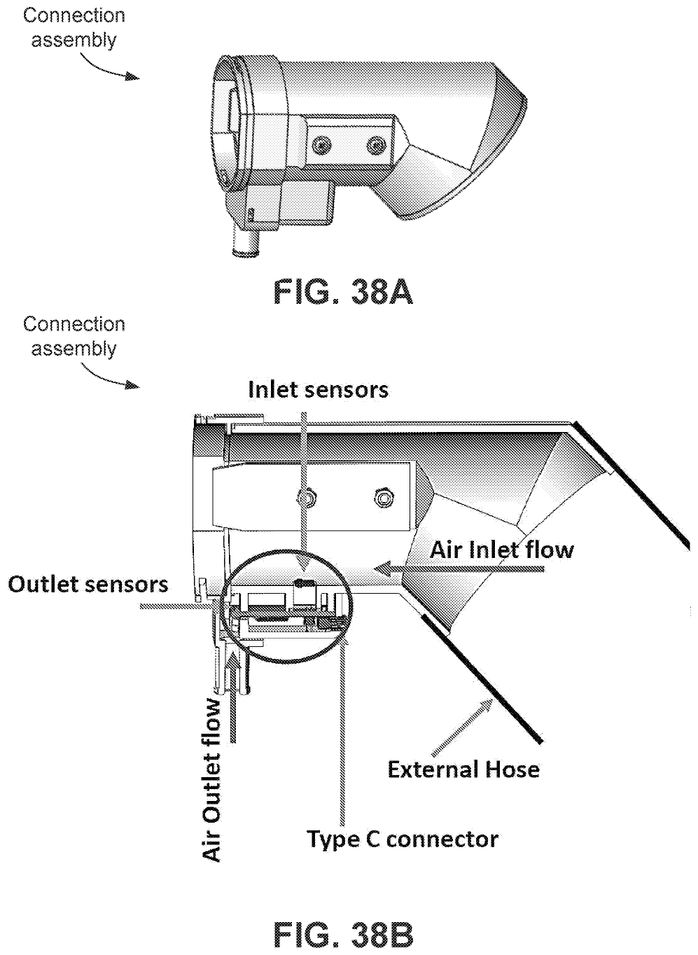

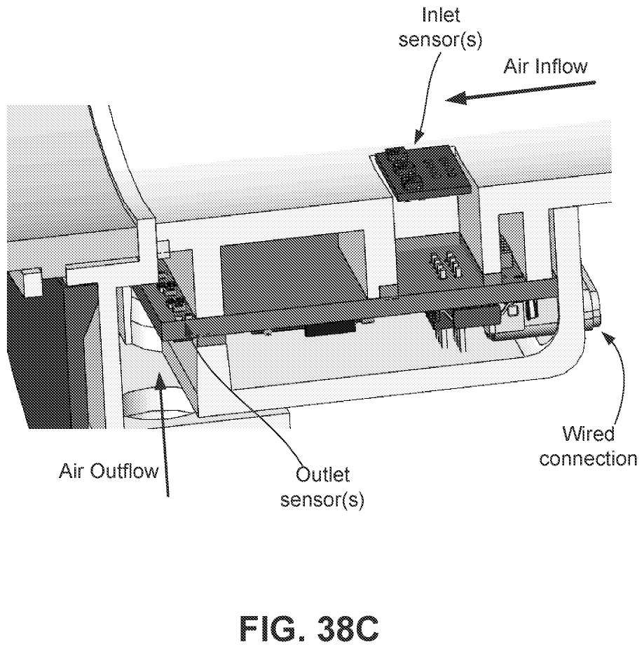

For example, FIGS. 38A, 38B, and 38C show an exemplary connection assembly for coupling an external hose (from the console unit) to the wearable treatment device and for placing one or more inlet temperature/humidity sensors and one or more outlet temperature/humidity sensors into respective flow paths of inflowing air and outflowing air relative to the inlet and outlet ports. As shown, the air flowing into the device from a console unit (i.e., and provided via a blower unit associated with the console unit) passes from an external hose through the connection assembly and passes over one or more inlet sensors (for measuring at least temperature and humidity of such air inflow). Also, as shown, an outlet provided on a leg of the wearable device may include a hose providing a pathway for outflowing air (i.e., air that has circulated within the environment within the device and passes out of the device through the outlet). The outflowing air may generally pass into the connection assembly and pass over one or more outlet sensors (for measuring at least temperature and humidity of such air outflow). Accordingly, as shown, the connection assembly includes an air inflow pathway for receiving incoming airflow, in which associated inlet temperature/humidity sensors are provided in said pathway, and an air outflow pathway for receiving outgoing airflow, in which associated outlet temperature/humidity sensors are provided in said pathway. Such a connection assembly can include any number of inlet and outlet sensors. For example, in one embodiment, the connection assembly may include three inlet sensors and three outlet sensors.

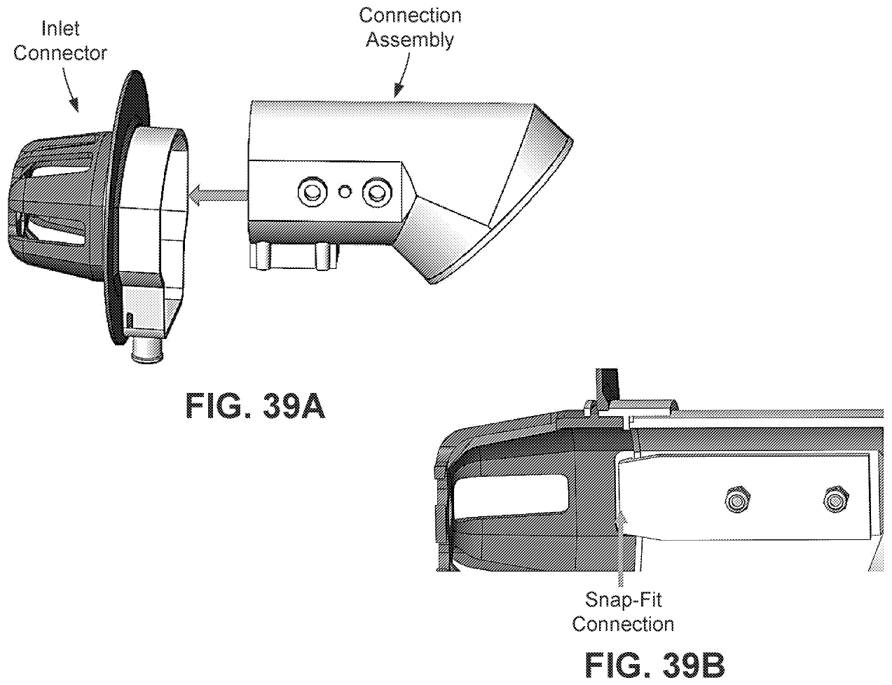

FIGS. 39A and 39B illustrate a snap-fit connection between the connection assembly and an inlet connector portion of the wearable treatment device.

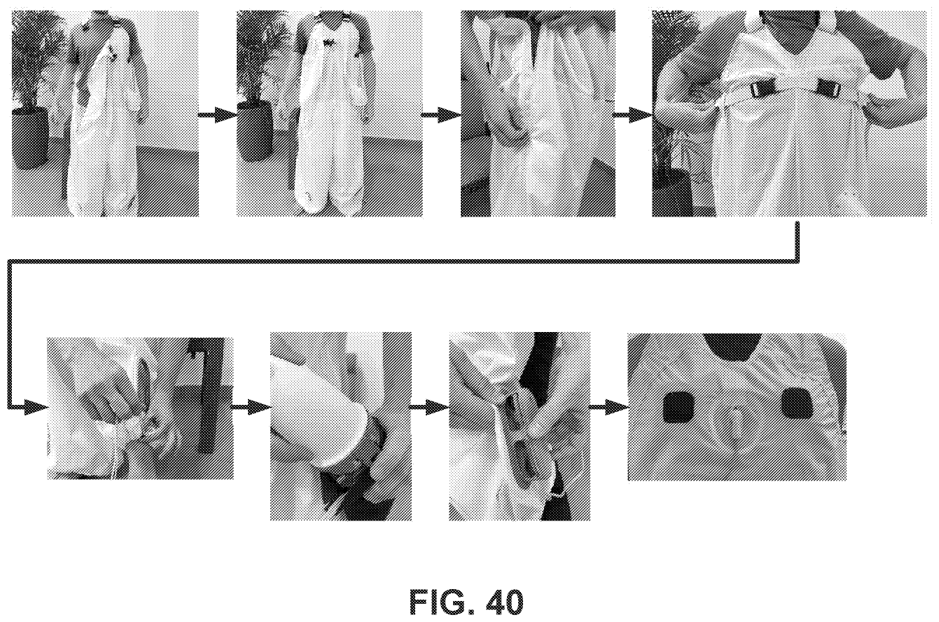

FIG. 40 shows a patient assembling the wearable treatment device. As shown, the device can transform from an unassembled state to an assembled state in a relatively simple fashion. For example, in the unassembled state, portions of the device are separated from one another to allow a patient to place the device over portions of their body. For example, a patient would first slip each leg into a respective leg of the device. Then, the patient need along place their arms through the respective shoulder straps (in which each shoulder strap is resting upon a respective shoulder. The device may have a single zipper along one side of the device (i.e., from about a waistline portion of the device up to an underarm portion of the device. Accordingly, the patient need only utilize the zipper to close the device around their body. Once a patient places the device onto the appropriate body parts, and zips up the zipper, the patient can then fasten the chest and shoulder straps, as well as the adjustable leg opening, so as to obtain a tight, yet comfortable fit. The patient and/or medical professional may connect the exterior hose (via the connection assembly) to the inlet port and further attach a hose from the outlet port to the connection assembly. The medical professional may further connect any additional sensors to the device and/or to the console unit.

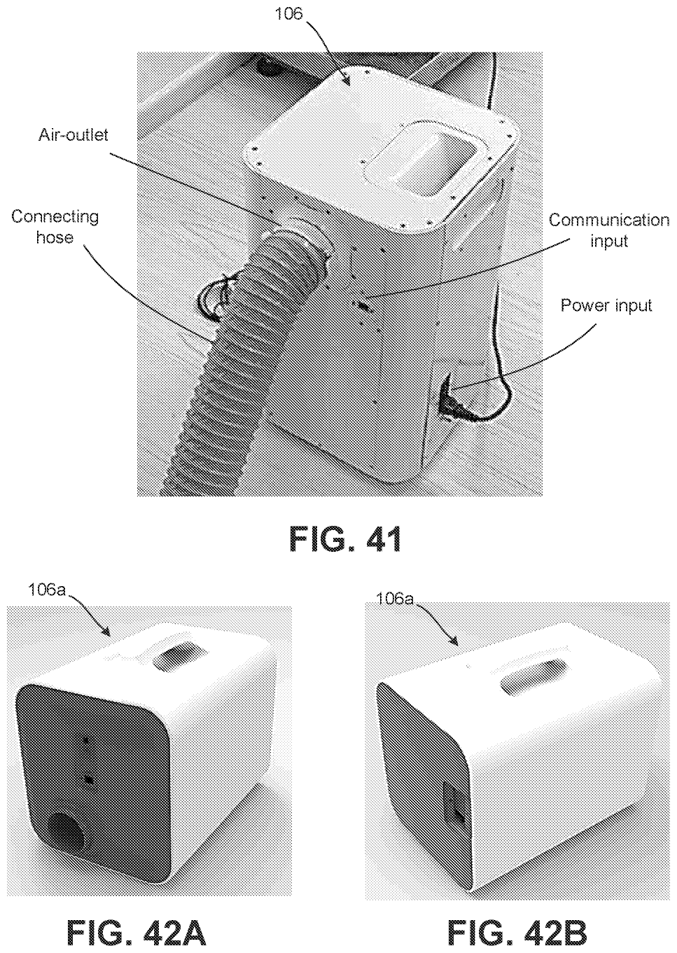

FIG. 41 is an image showing an exemplary embodiment of a console unit 106 consistent with the present invention. FIGS. 42A and 42B are images showing front and rear views of an alternative embodiment of a console unit 106a consistent with the present disclosure. As illustrated, the console unit 106 generally includes an air-outlet through which heated air is delivered (through the connected hose to the treatment device 102), a power input allowing for the console unit 106 to be plugged into an electric source, and a communication input (i.e., ethernet or other data communication input port). It should be noted that, in some embodiments, the console unit 106 may include a rechargeable power source (i e., rechargeable battery (ies)), thereby increasing mobility of the system 100. Furthermore, in some embodiments, the system may include a docking station into which the console unit 106 may be placed for recharging (i.c., wheeled console unit 106 can be pushed into docking station).

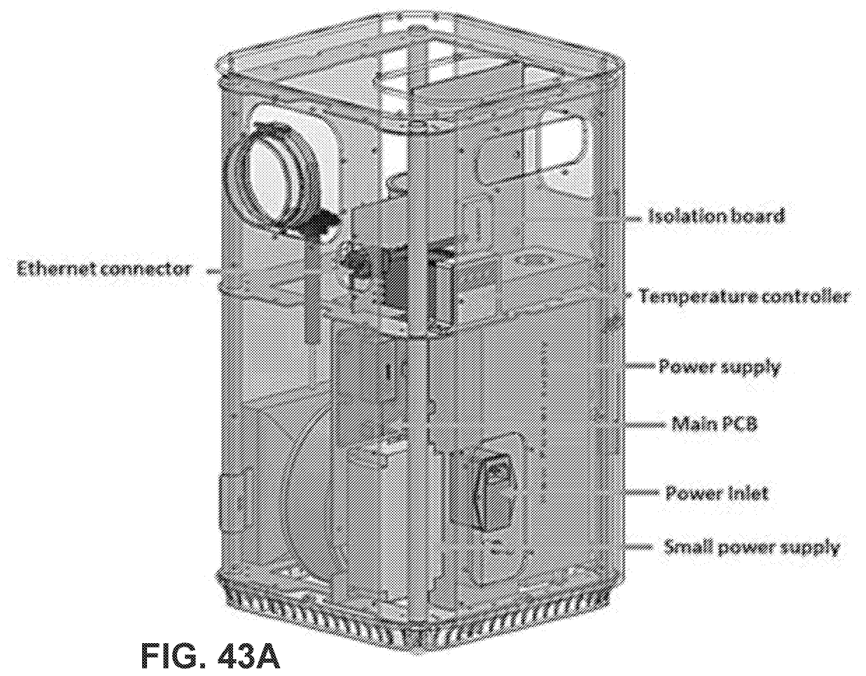

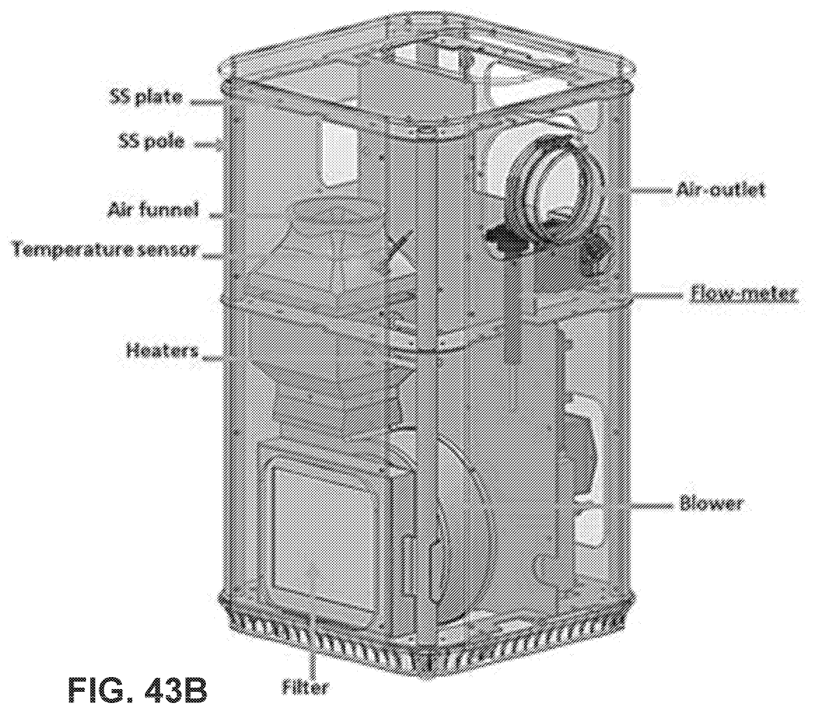

FIGS. 43A and 43B show front and rear perspective views, partly in section, of the console unit 106 illustrating various components of the console unit 106. The unit generally 106 consists of a HEPA filter (0.3-1 micron particles level), a blower unit, two heaters, and a temperature sensor, including a temperature controller. The unit 106 is a hand-carried, portable device. The output air temperature may be controlled based, at least in part, on temperature measurements obtained via a temperature sensor located at the inlet port of the treatment device 102. For example, a controller 110 may compare the output air temperature to the inlet temperature sensor and, using an on-off control scheme, turns the heater(s) on or off, accordingly until a desired temperature is reached and maintained.

The console unit 106 is designed to supply an airflow of 1-2 cubic meters/min to the treatment device 102 and to maintain the wearable contact surface average temperature between 38° C.-45° C. The air flow may be measured with a flow meter (F300, Degree Controls) located at the air-outlet of the console unit 106. Treatment is initiated when the patient puts on the treatment device and connects the air flow hose. Upon operation, warm air is administered from the console unit 106 to the treatment device 102, which results in the creation of a warm, relatively dry air environment within the treatment device 102, with an average air temperature ranging from 38° C.-48° C. and relative humidity lower than 60%, more preferably lower than 50%. The warm air exists the treatment device through the outlet port(s) located at the legs region.

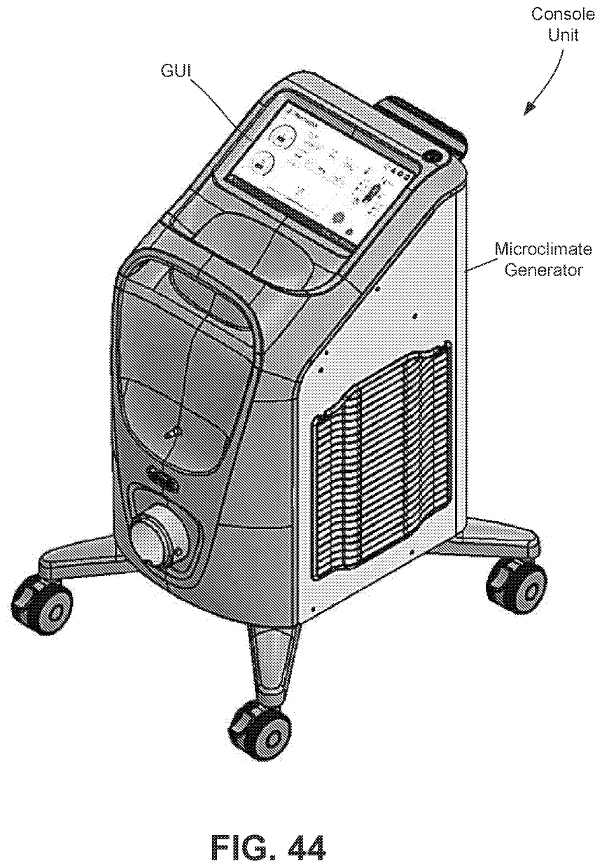

FIG. 44 is a perspective view of an exemplary console unit used with the system of the present disclosure. As shown, the console unit is a mobile unit (via a set of legs and wheels), thereby improving the mobility of the system of the present disclosure.

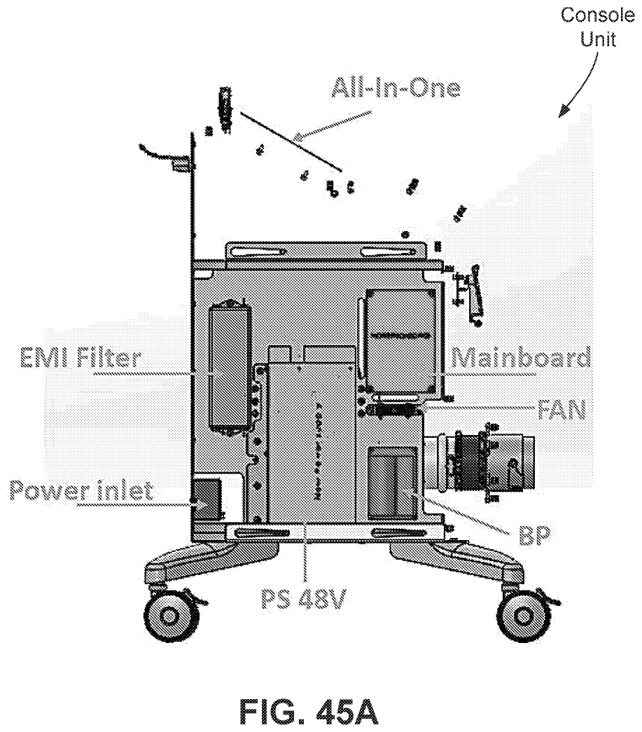

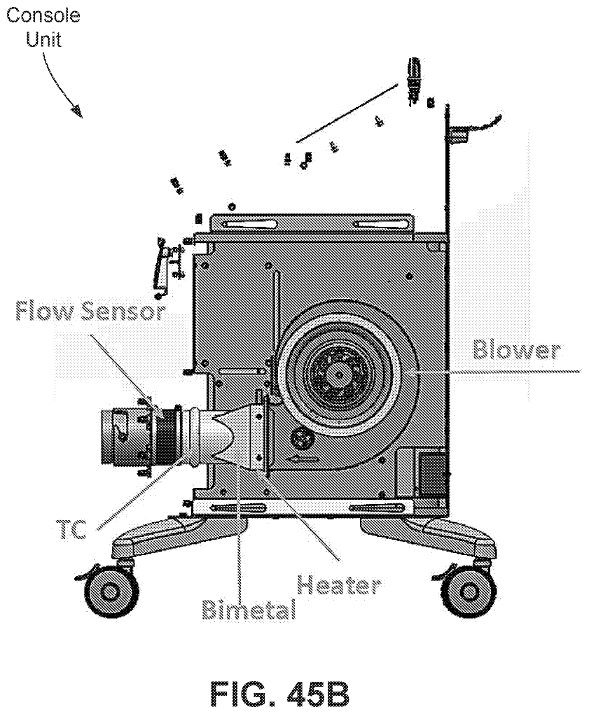

FIGS. 45A and 45B are side views of the console unit illustrating various internal components, including electrical and mechanical components. The console unit generally includes an air-outlet through which heated air is delivered (through the connected hose to the treatment device), a power input allowing for the console unit to be plugged into an electric source, and a communication input (i.e., ethernet or other data communication input port, such as a SIM card input, USB input, or the like). It should be noted that, in some embodiments, the console unit may include a rechargeable power source (i.e., rechargeable battery(ies)), thereby increasing mobility of the system 100. Furthermore, in some embodiments, the system may include a docking station into which the console unit may be placed for recharging (i.e., wheeled console unit can be pushed into docking station). The console unit generally further consists of a HEPA filter (0.3-1 micron particles level), a blower unit, a heater, a flow sensor, and a temperature sensor (thermocouple), including a temperature controller. The output air temperature may be controlled based, at least in part, on temperature measurements obtained via a temperature sensor located at the inlet port of the wearable treatment device 102. For example, a controller of the console unit may compare the output air temperature to the inlet temperature sensor and, using an on-off control scheme, turns the heater on or off, accordingly until a desired temperature is reached and maintained. In other embodiments, as described in greater detail herein, the console may utilize a proportional-integral-derivative (PID) controller for controlling the heater, as opposed to an on-off control scheme.

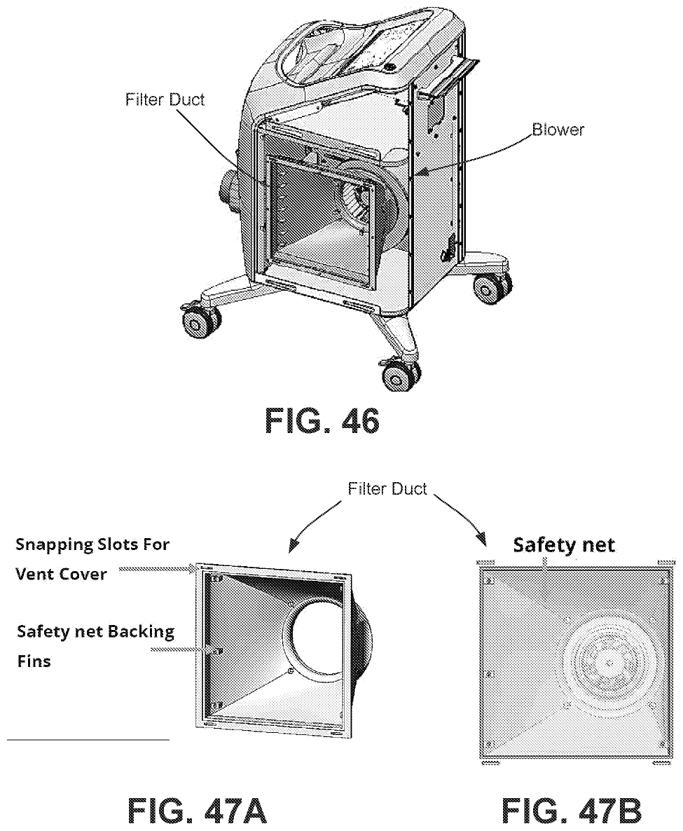

FIG. 46 is a perspective view of the console unit illustrating portions of the airway assembly, including a filter duct and blower fan. FIGS. 47A and 47B show a filter duct. The filter duct is generally coupled to the blower fan and configured to receive ambient air which is drawn in via operation of the blower fan and redirected toward the heating assembly and subsequently out of the air-outlet of the console unit, through the hose, and into the wearable treatment device via the inlet port of the device. In an assembled state, a filter is place in line with an opening of the filter duct to thereby filter out any contaminants from the intake.

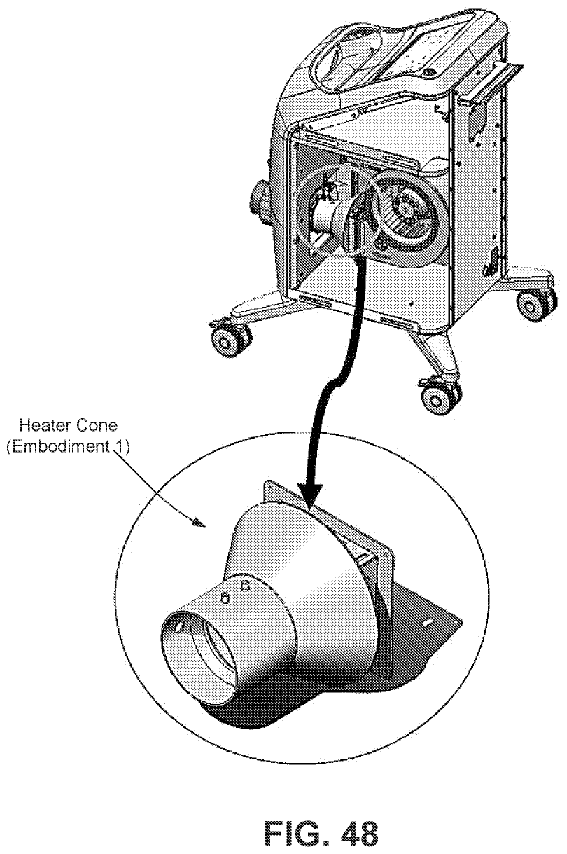

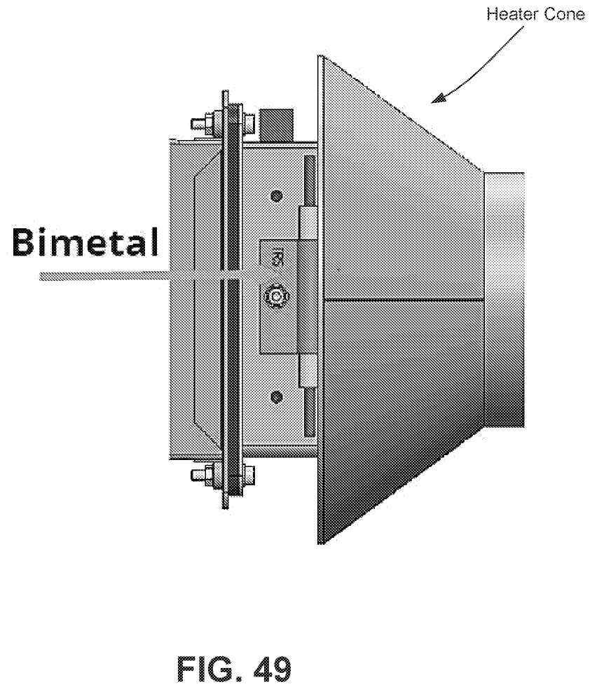

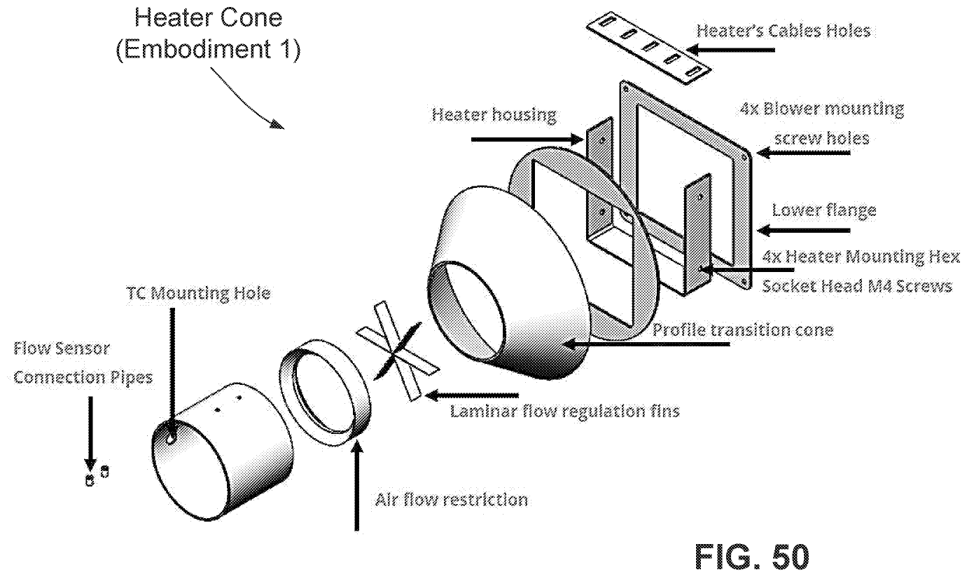

FIG. 48 is a perspective view of the console unit illustrating one embodiment of a heater cone component in greater detail. FIG. 49 is side view of the heater cone coupled to a heater member. FIG. 50 is an exploded perspective view of the heater cone of FIG. 48.

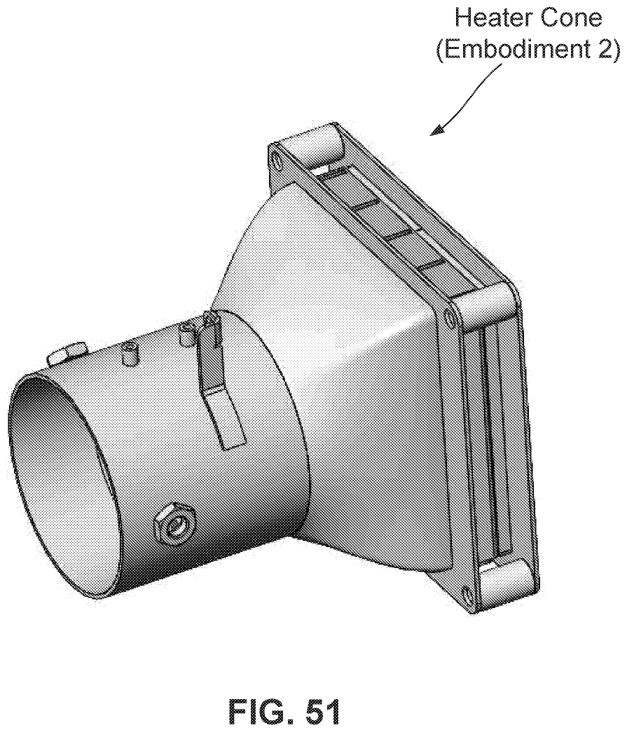

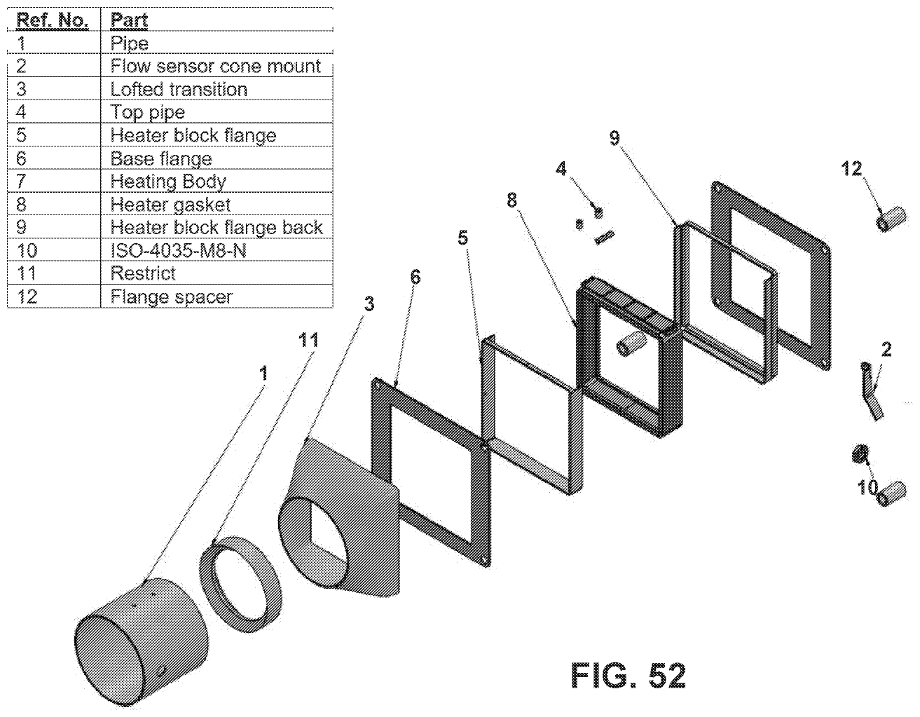

FIG. 51 is a perspective view of another embodiment of a heater cone component consistent with the present disclosure. FIG. 52 is an exploded perspective view of the heater cone of FIG. 51.

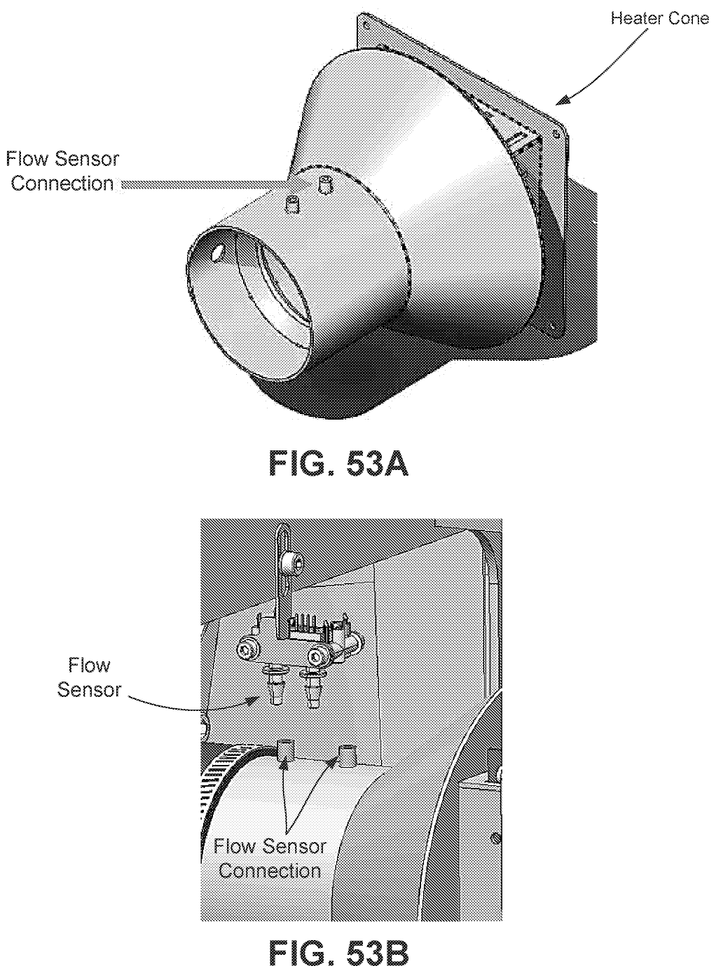

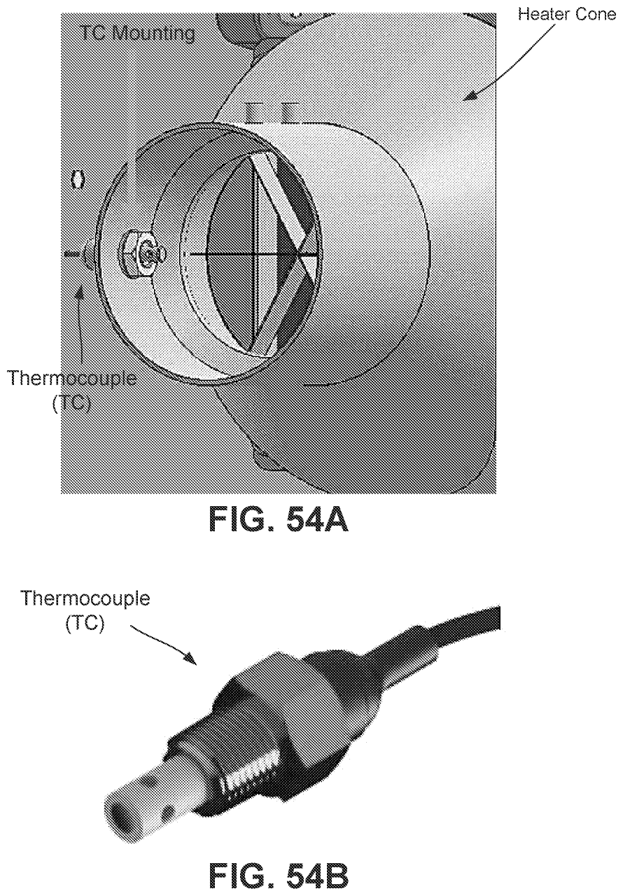

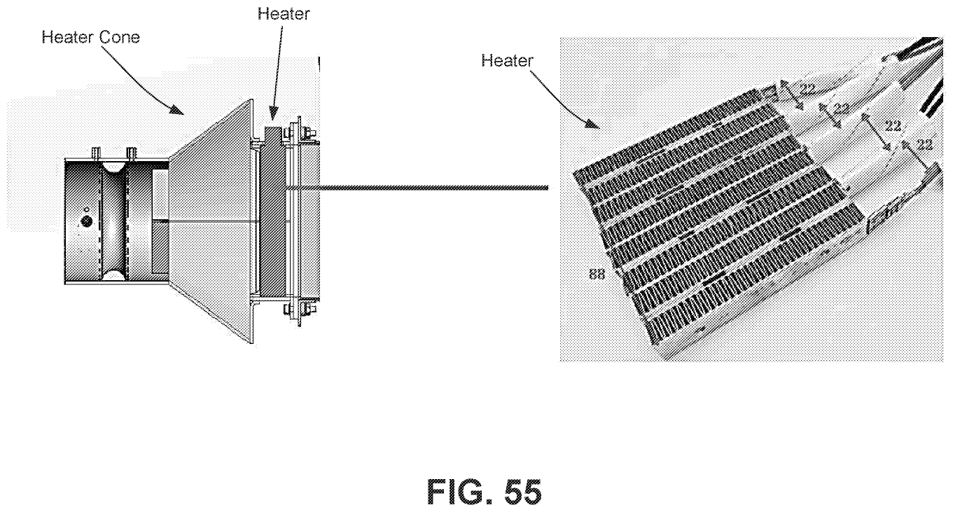

FIG. 53A is a perspective view of the heater cone illustrating a flow sensor connection portion. FIG. 53B shows placement of the flow sensor relative to the corresponding flow sensor connection of the heater cone. FIG. 54A shows mounting of a thermocouple to a respective portion of the heater cone. FIG. 54B shows an exemplary thermocouple. FIG. 55 is a side view of the heater cone coupled to a heater

The console unit is designed to supply an airflow of 1-2 cubic meters/min to the wearable treatment device via a blower (SE-F160C-EC-01 DC Forward Centrifugal Fan, offered by Blauberg Motoren) and to maintain the wearable contact surface average temperature between 38° C.-45° C. The air flow may be measured with a flow sensor (SDP810-Digital, offered by Sensirion) located at the air-outlet of the console unit. Treatment is initiated when the patient puts on the treatment device and connects the air flow hose. Upon operation, warm air is administered from the console unit 106 to the wearable treatment device, which results in the creation of a warm, relatively dry air environment within the wearable treatment device, with an average air temperature ranging from 38° C.-48° C. and relative humidity lower than 60%, more preferably lower than 50%. The warm air exists the treatment device through the outlet port(s) located at the legs region.

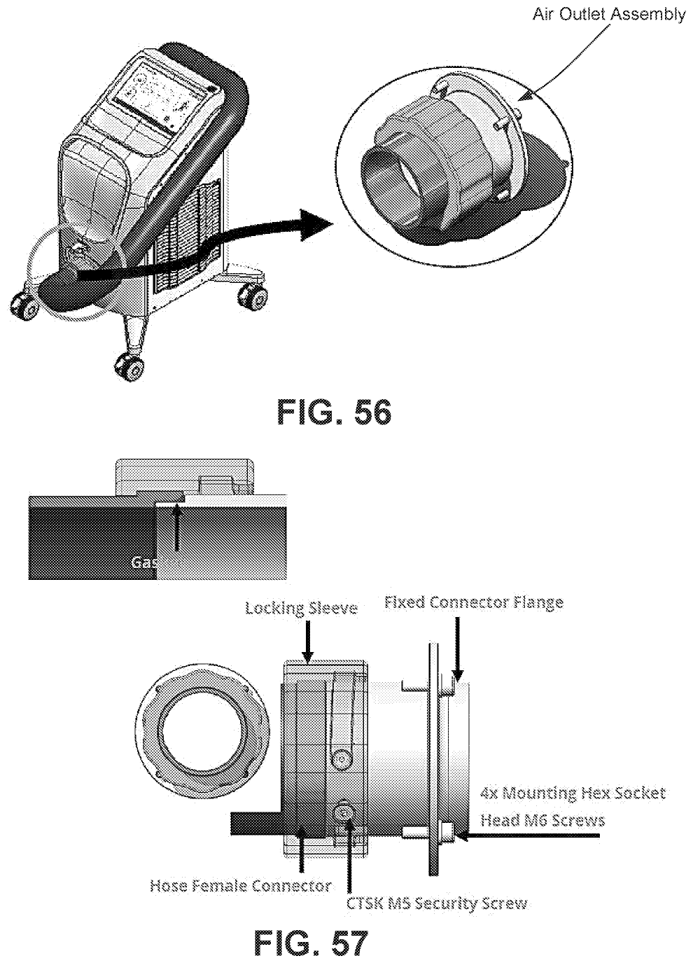

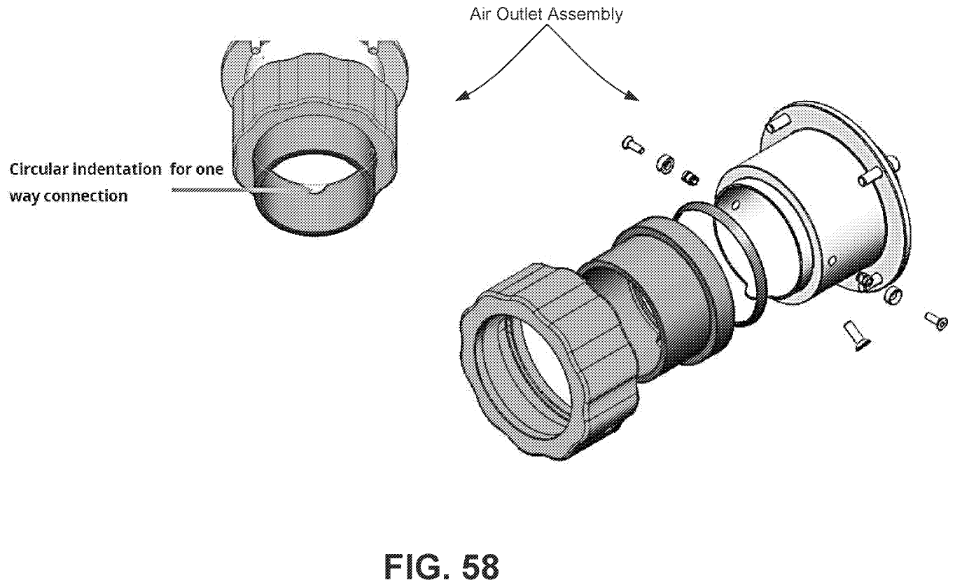

FIG. 56 shows an air outlet assembly for coupling one end of a hose to the console unit. FIG. 57 illustrates components of the air outlet assembly in greater detail. FIG. 58 is an exploded perspective view illustrating components of the air outlet assembly in greater detail.

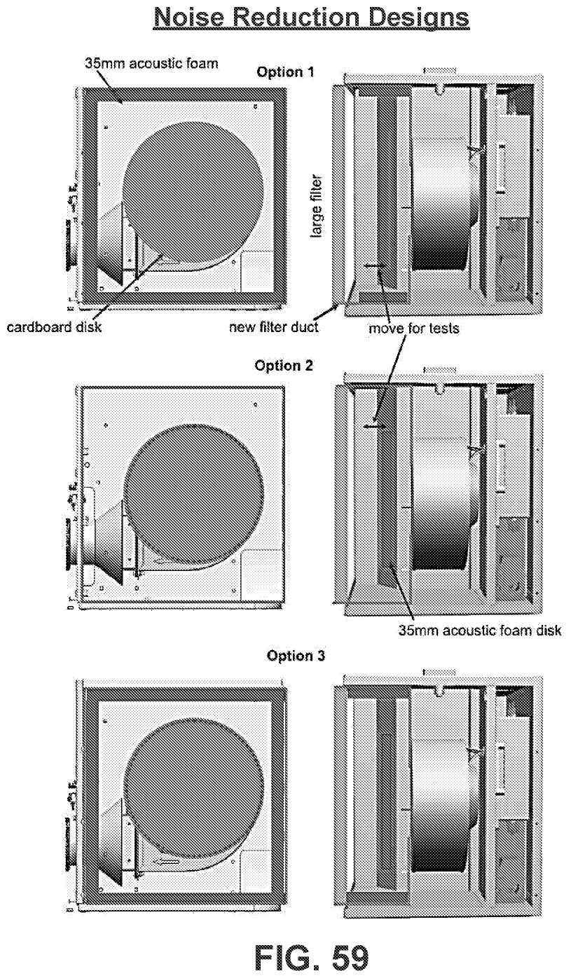

In an effort to reduce overall noise during operation of various components of the console unit, most notably the blower fan, different noise reduction designs were contemplated. FIG. 59 shows three different embodiments of noise reducing designs for reducing overall noise of the console unit, particularly reducing noise caused during operation of the blower fan. In a first design option, acoustic foam was placed along a perimeter of the filter duct and a cardboard disk was placed in coaxial alignment with the intake of the blower fan. In a second design option, a cardboard disk was placed in coaxial alignment with the intake of the blower fan and an acoustic foam disk was also placed in coaxial alignment with the intake of the blower fan. In a third design option, acoustic foam was placed along a perimeter of the filter duct and both a cardboard disk and an acoustic foam disk were placed in coaxial alignment with the intake of the blower fan.

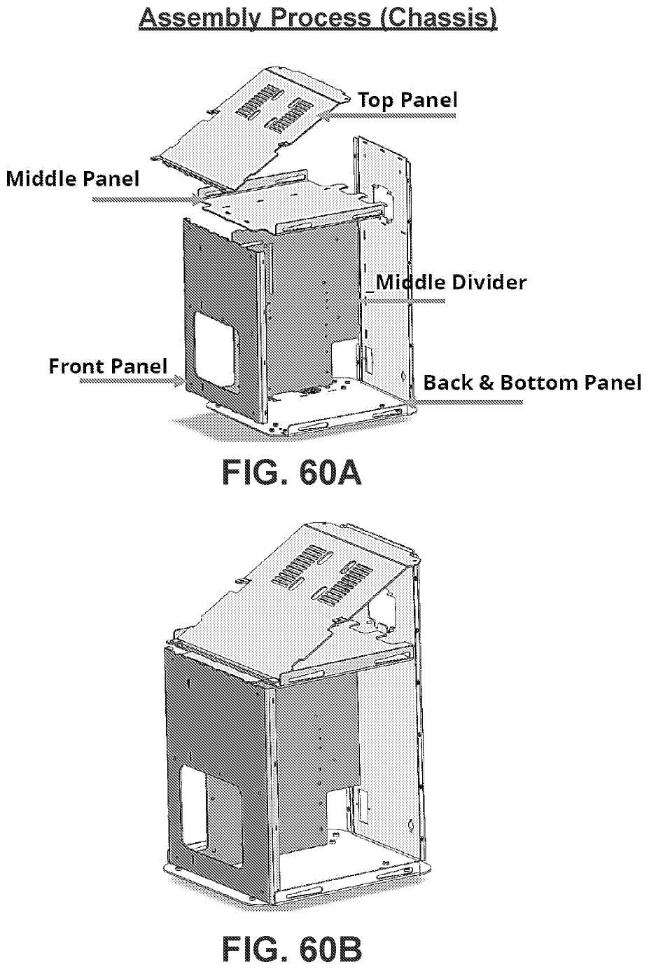

FIGS. 60A and 60B show an exploded and assembled view, respectively, of the components forming a chassis of the console unit.

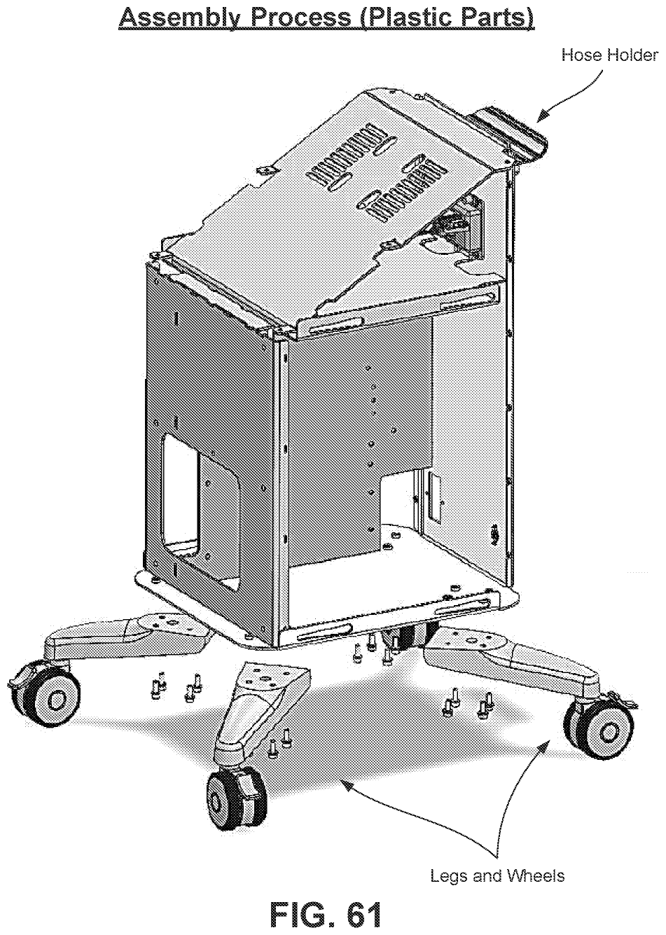

FIG. 61 shows a partially exploded view illustrating placement of legs and wheels, as well as a hose holder, upon the assembled chassis.

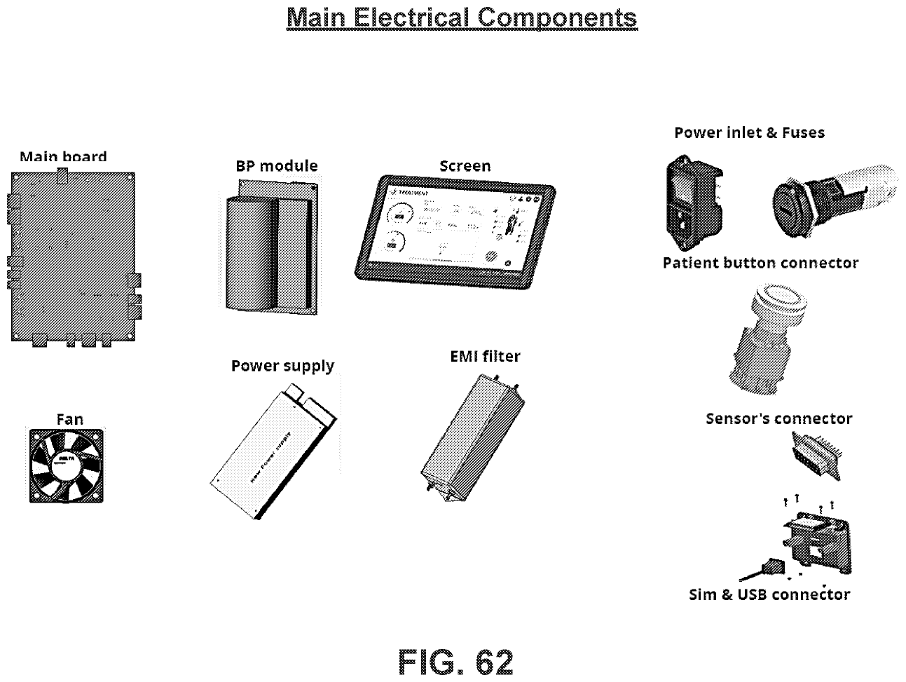

FIG. 62 shows the various main electrical components provided in the console unit.

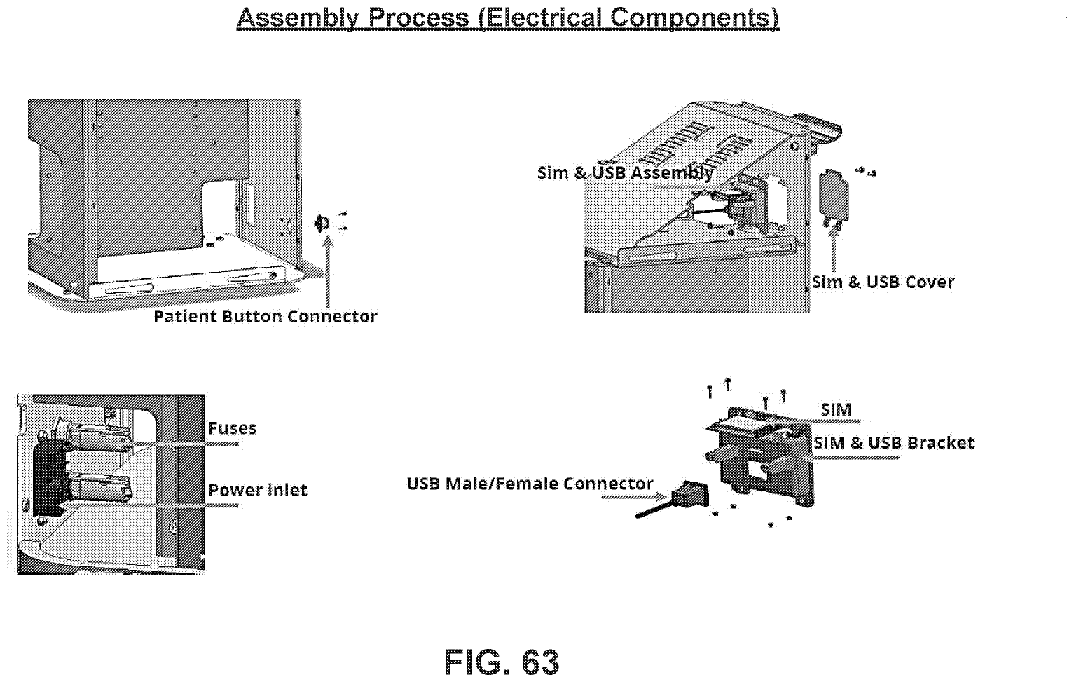

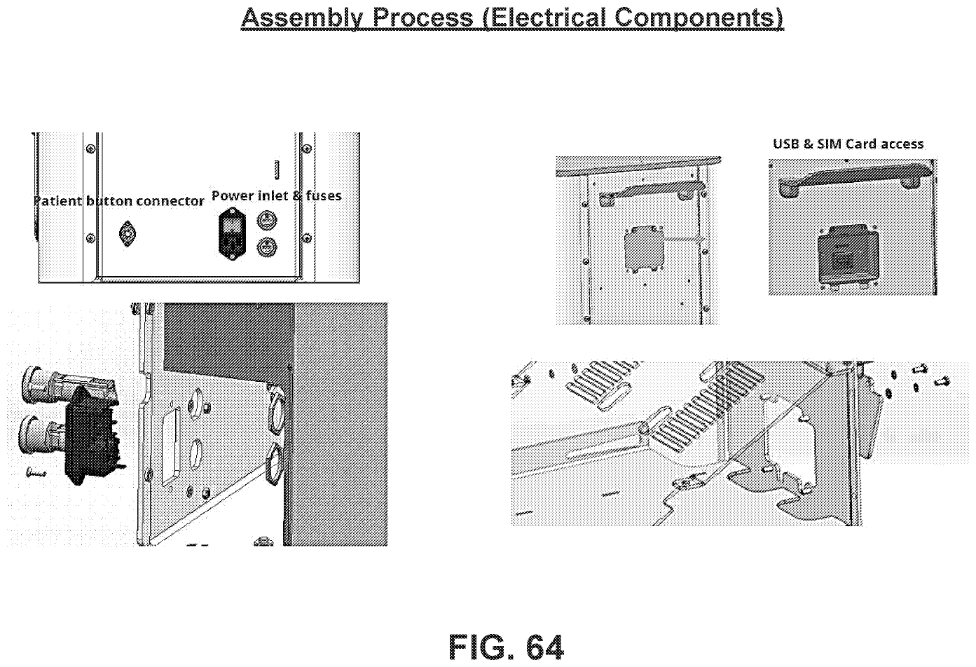

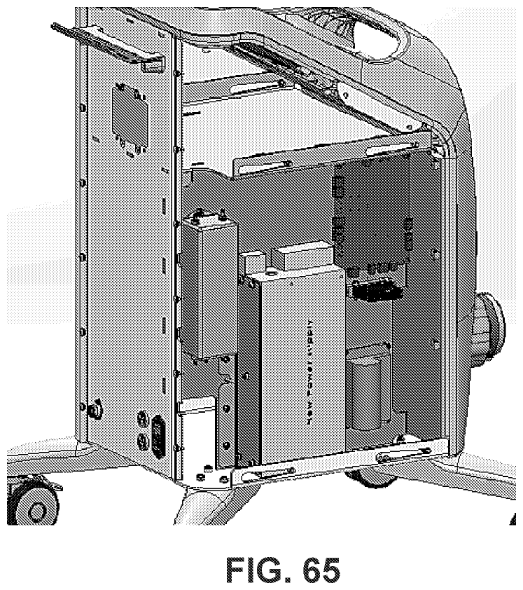

FIGS. 63, 64, and 65 illustrate various assembly processes for assembling electrical components to the chassis of the console unit.

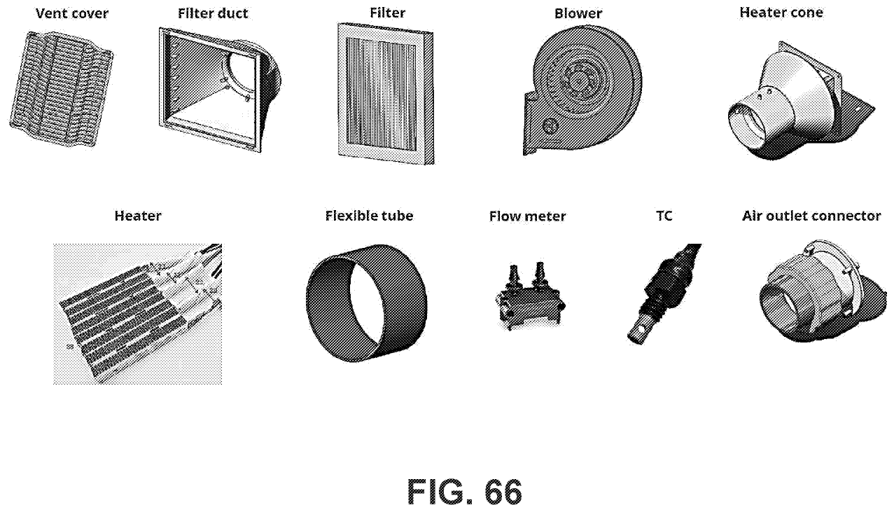

FIG. 66 shows the various airway assembly components provided in the console unit.

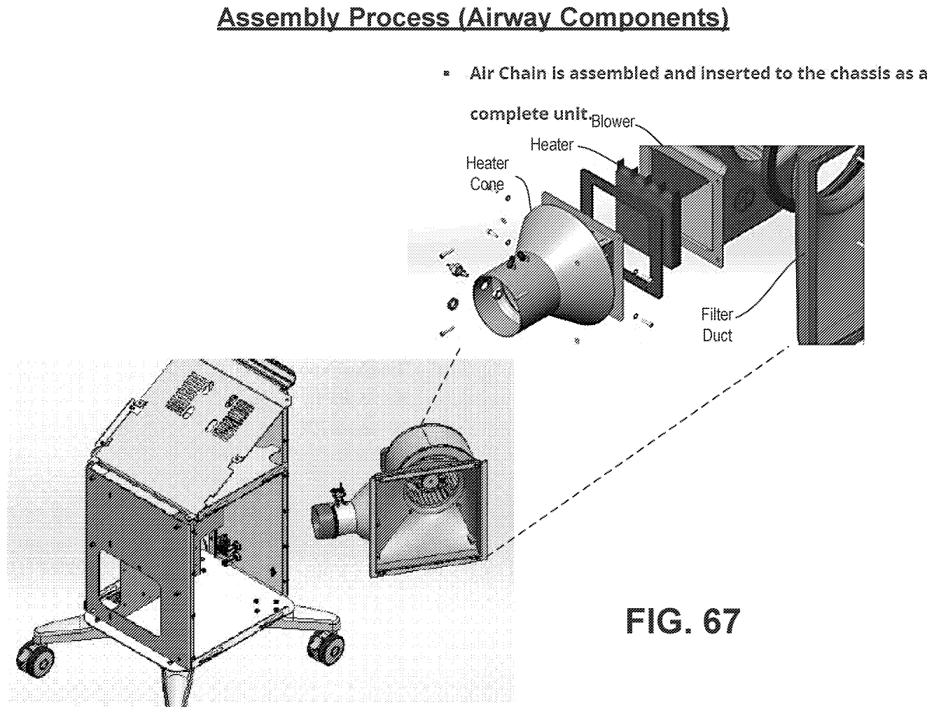

FIG. 67 shows assembly of the airway components and placement within the chassis of the console unit.

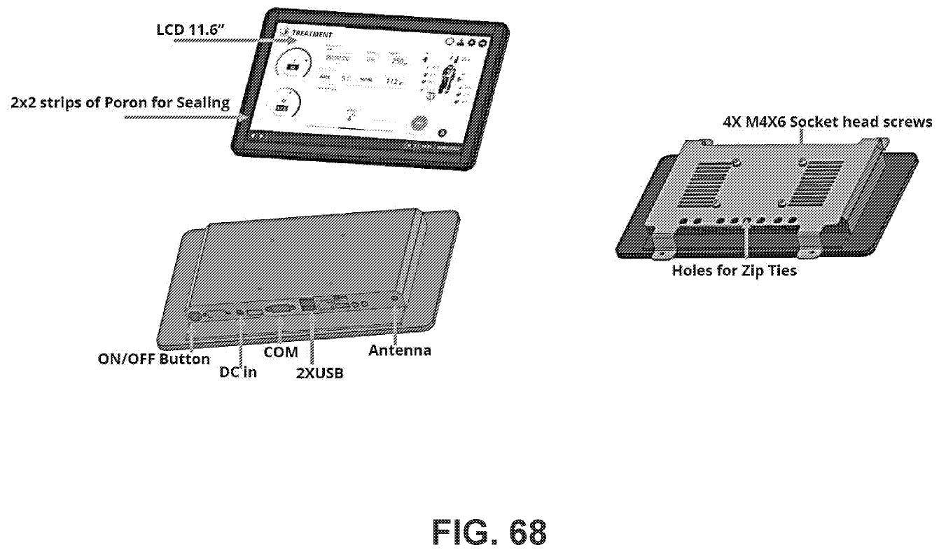

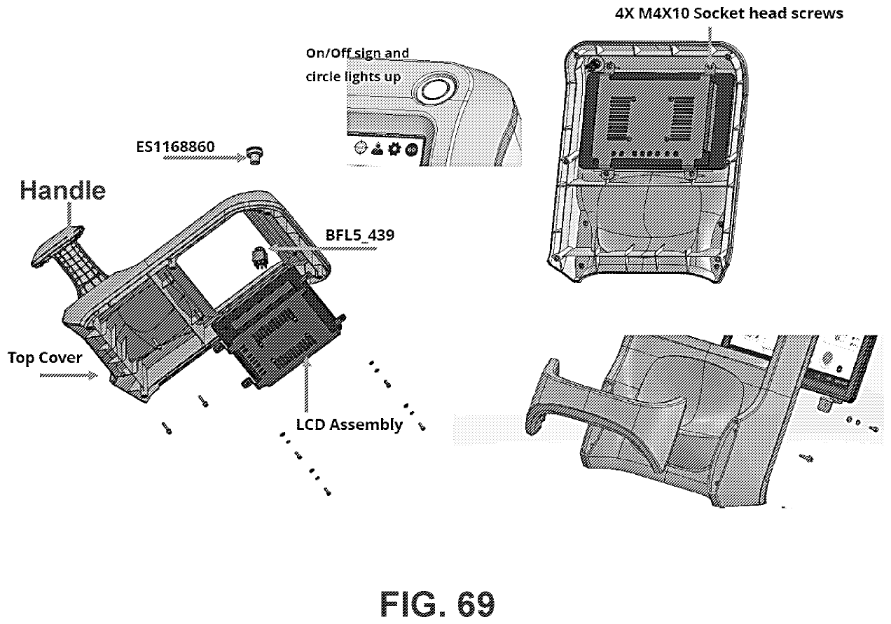

FIGS. 68 and 69 show various assembly processes for assembly the top cover and associated components, including placement of the controller with touchscreen display, to the chassis of the console unit.

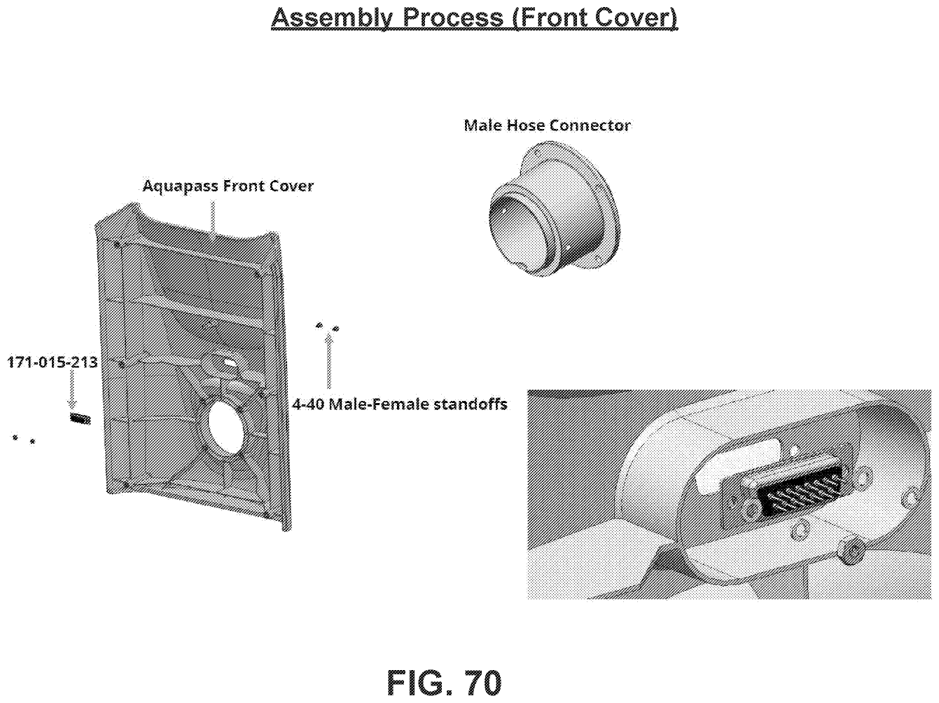

FIG. 70 shows assembly processes for coupling the front cover and associated components to the chassis of the console unit.

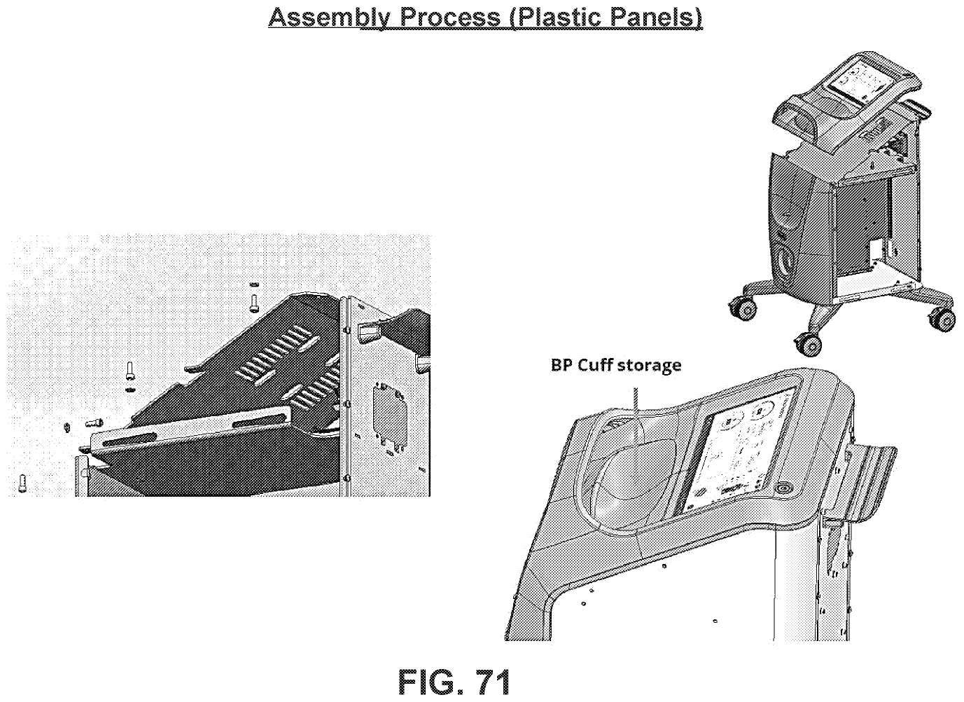

FIG. 71 shows assembly processes for coupling plastic panels to the chassis of the console unit.

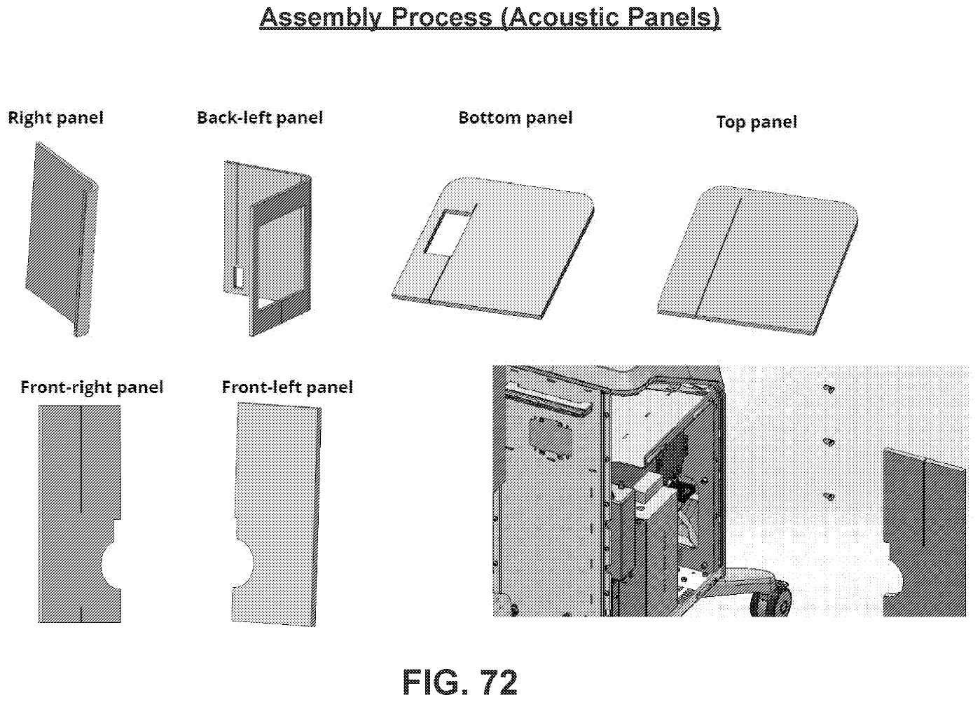

FIG. 72 shows various acoustic panels to be placed relative to the chassis of the console unit and provide noise reduction.

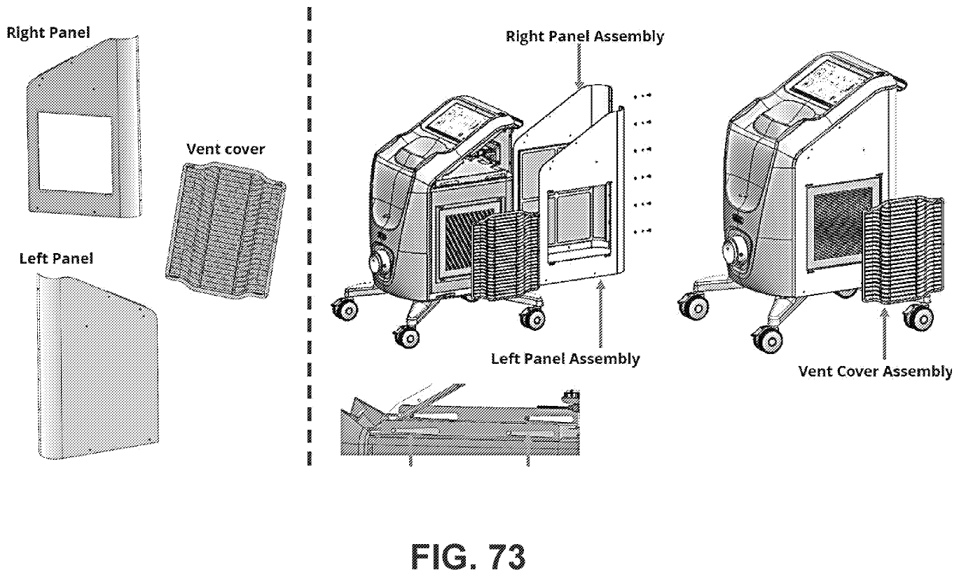

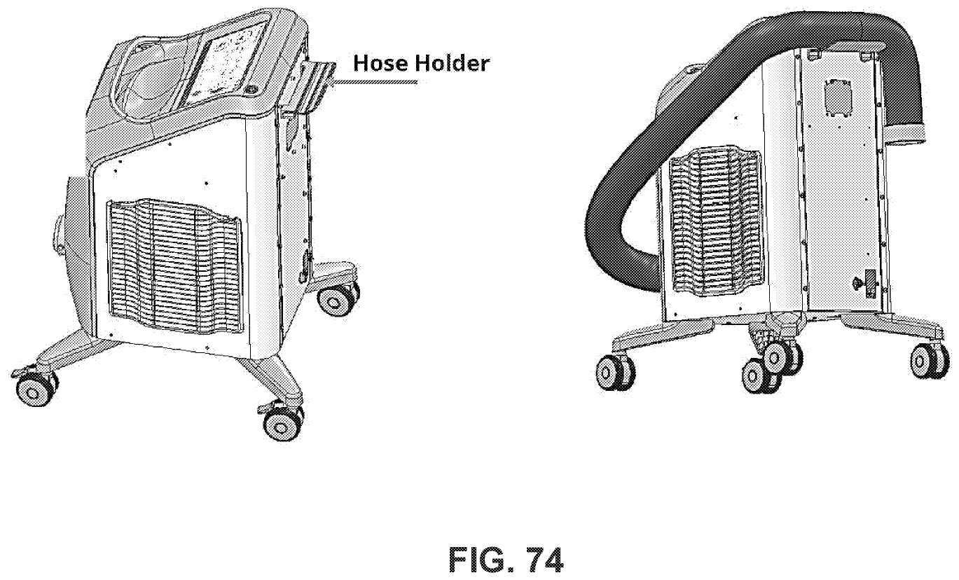

FIG. 73 shows assembly processes for coupling exterior paneling to the chassis of the console unit and other portions thereof.

FIG. 74 shows a fully assembled console unit, including a hose holder for retaining a loose, unconnected portion of the hose (when the hose is uncoupled from a wearable treatment device and not in use).

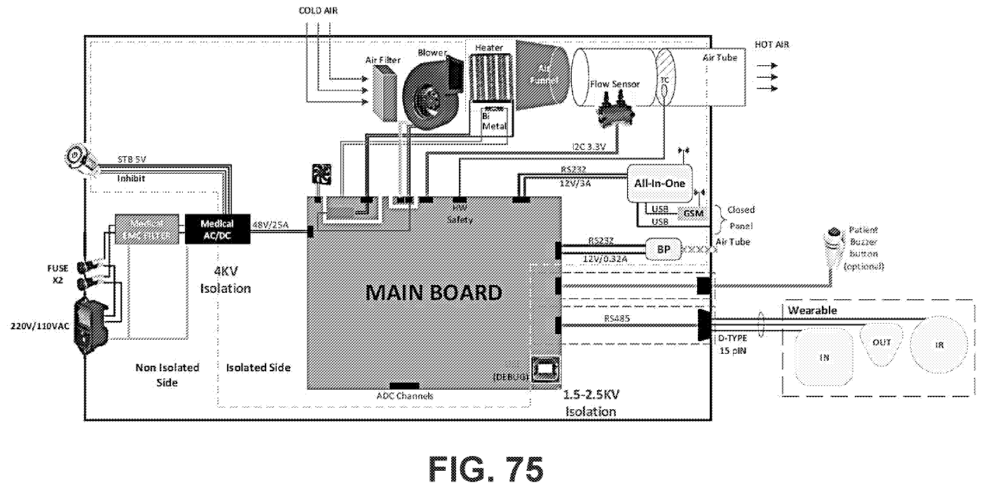

FIG. 75 is a schematic block diagram of the console unit architecture.

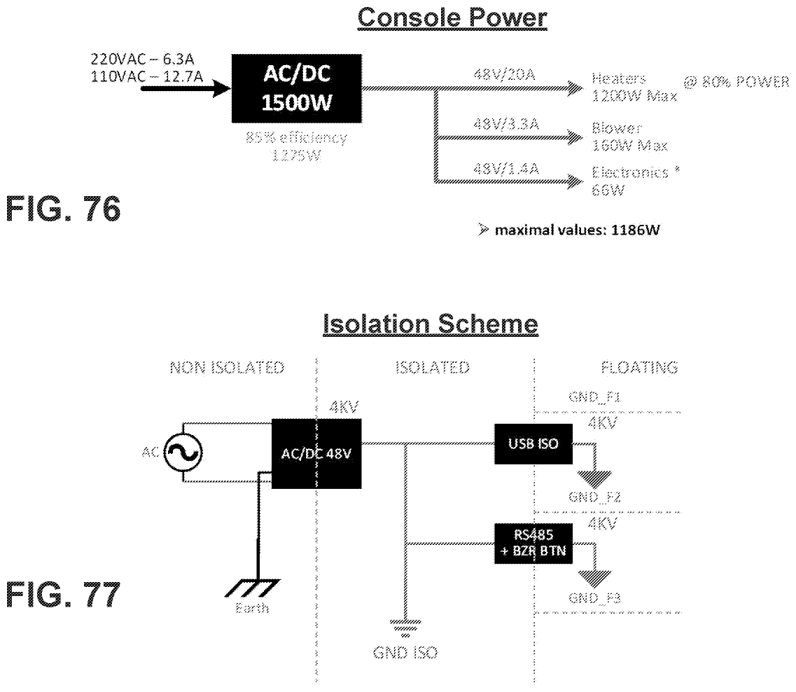

FIG. 76 is a schematic block diagram one embodiment of console unit power supply scheme.

FIG. 77 is a circuit diagram representing one embodiment of an isolation scheme.

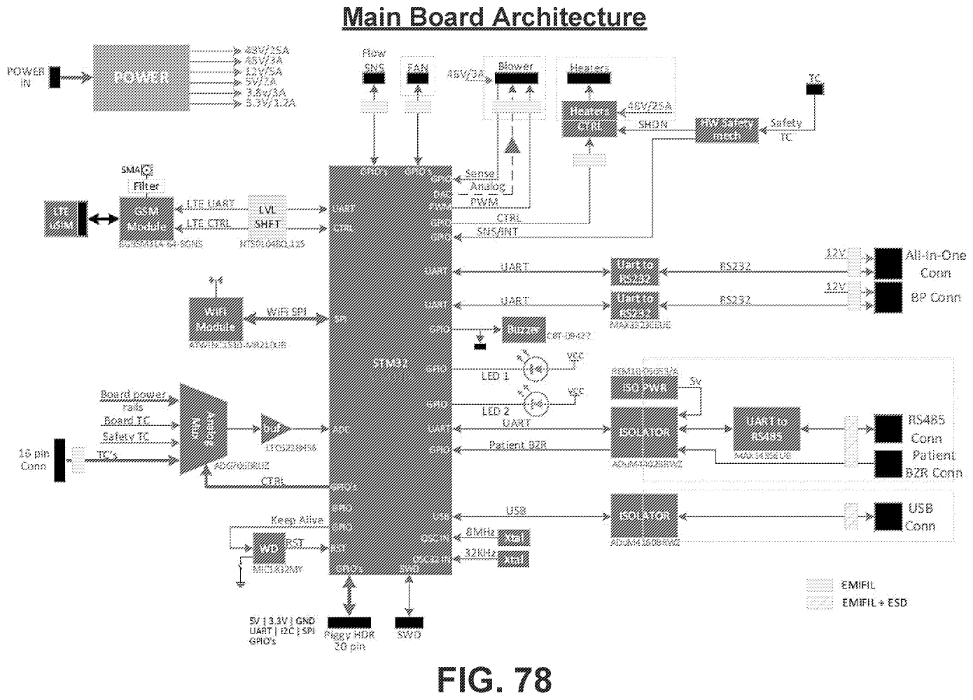

FIG. 78 is a schematic block diagram of the main board architecture.

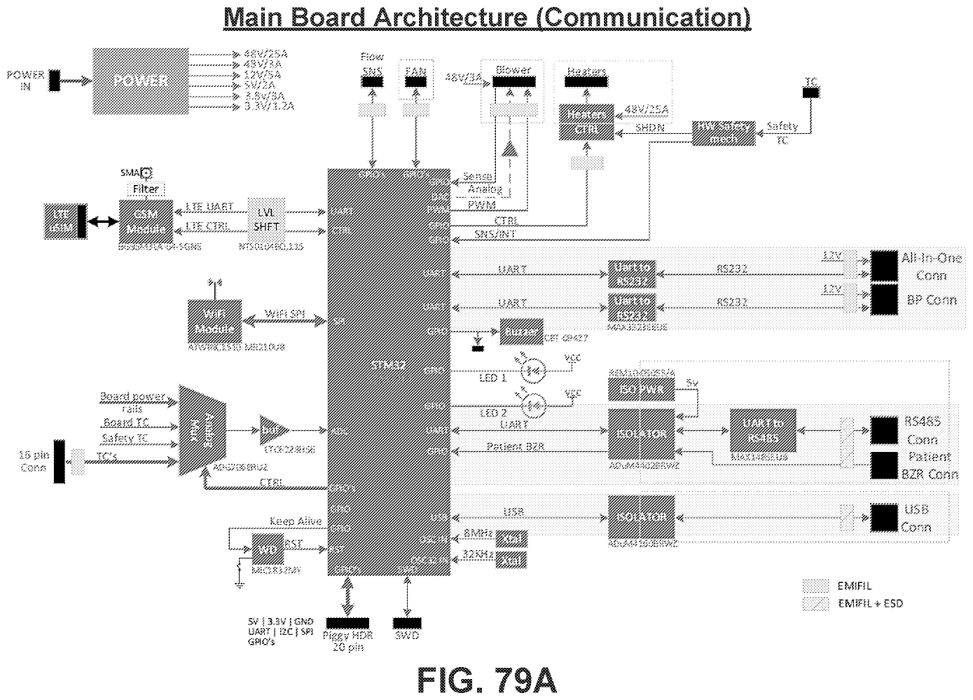

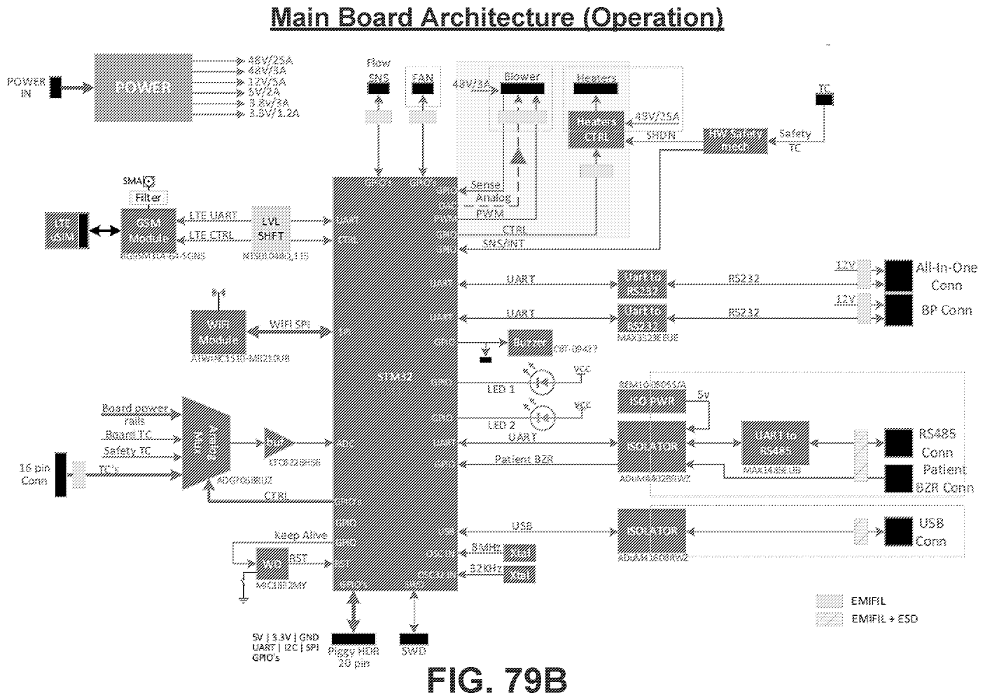

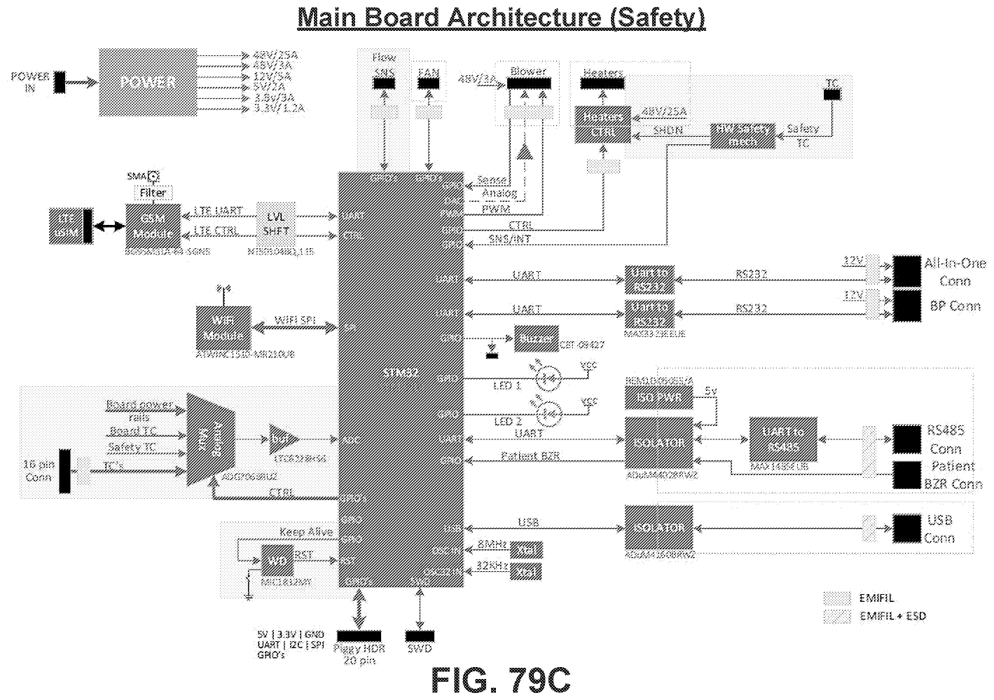

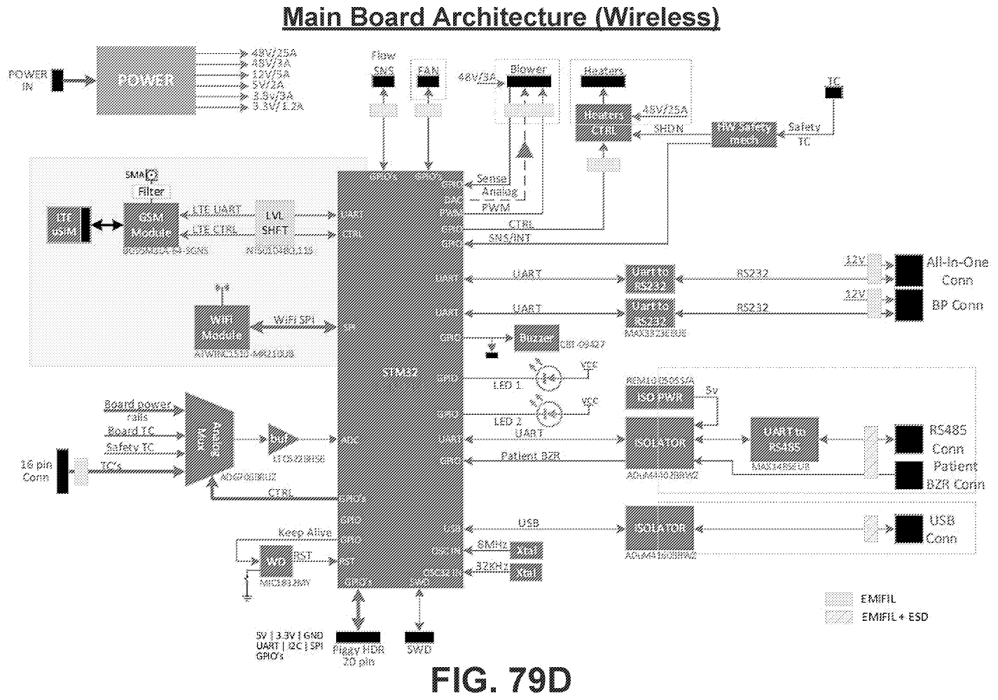

FIGS. 79A, 79B, 79C, and 79D highlight communication, operation, safety, and wireless aspects of the schematic block diagram the main board architecture of FIG. 78.

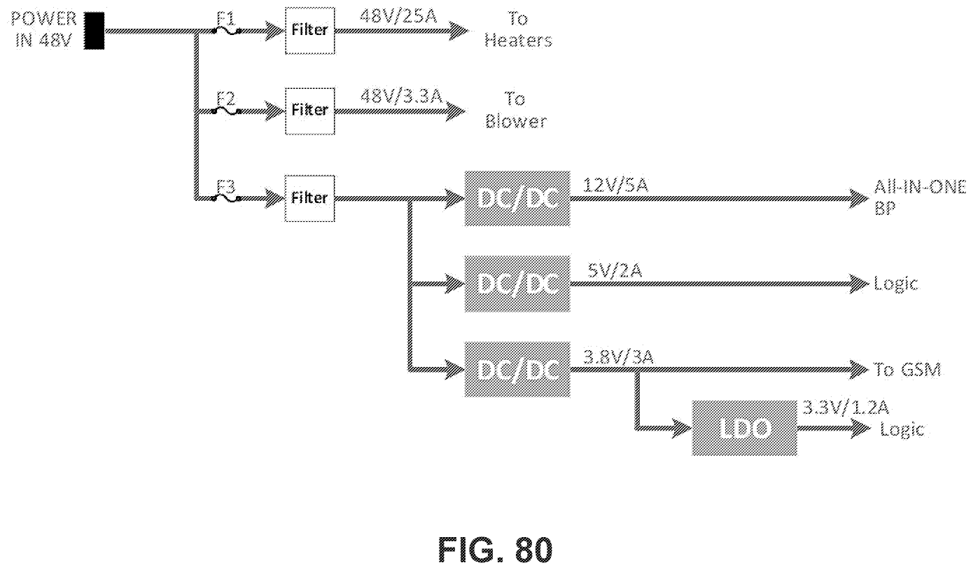

FIG. 80 is a schematic block diagram of the main board power supply scheme.

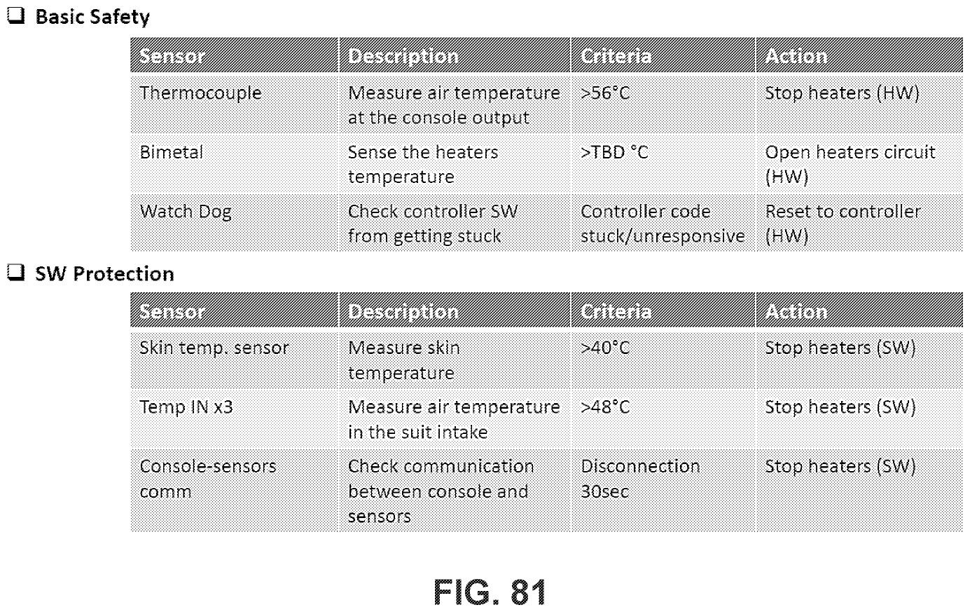

FIG. 81 is a chart describing various safety features utilized by the console unit of the present disclosure.

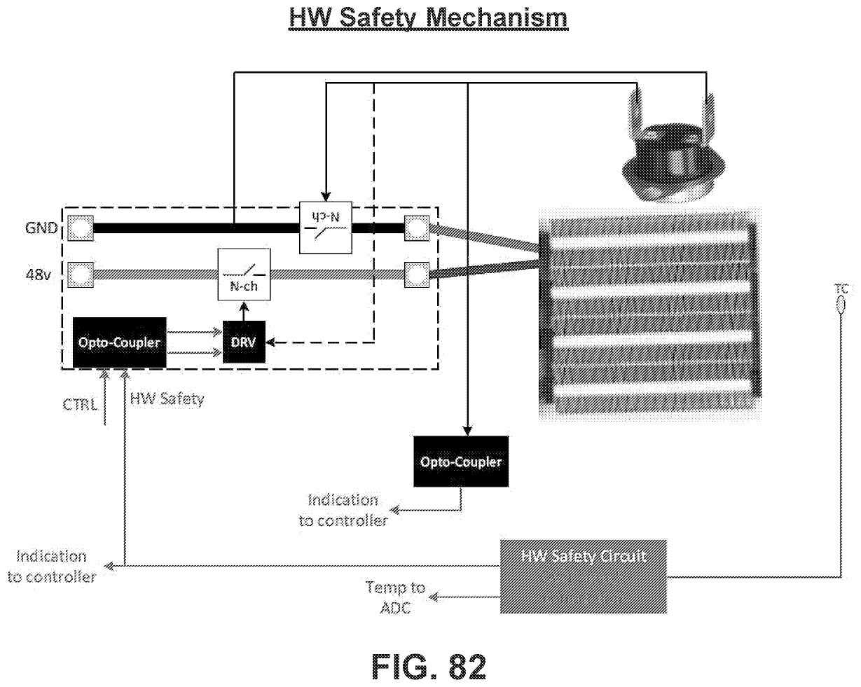

FIG. 82 is a schematic block diagram illustrating one embodiment of a HW safety mechanism.

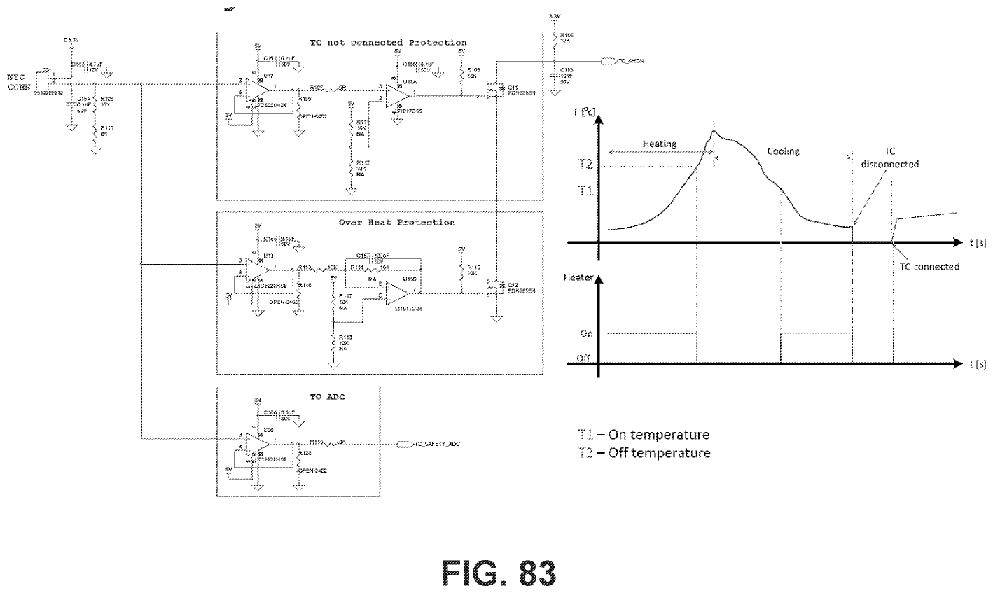

FIG. 83 is a circuit diagram representing one embodiment of a HW safety circuit.

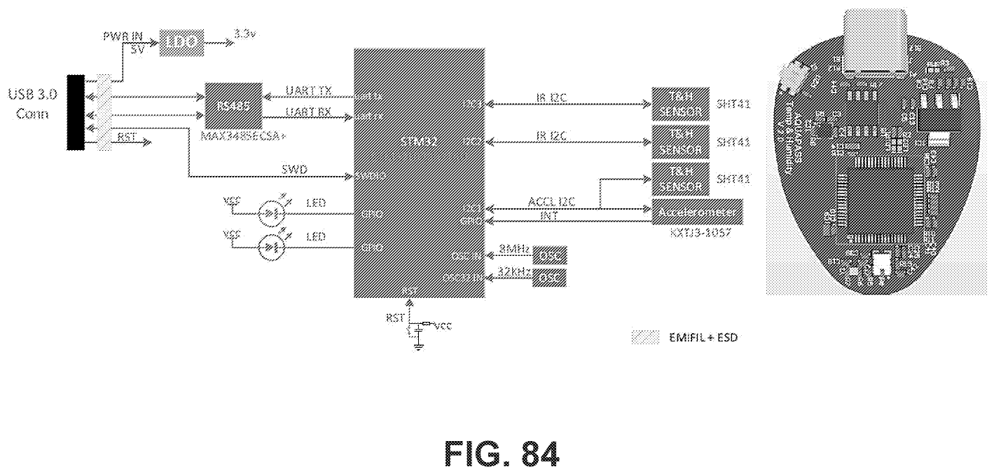

FIG. 84 is a circuit diagram representing one embodiment of a temperature/humidity sensor architecture.

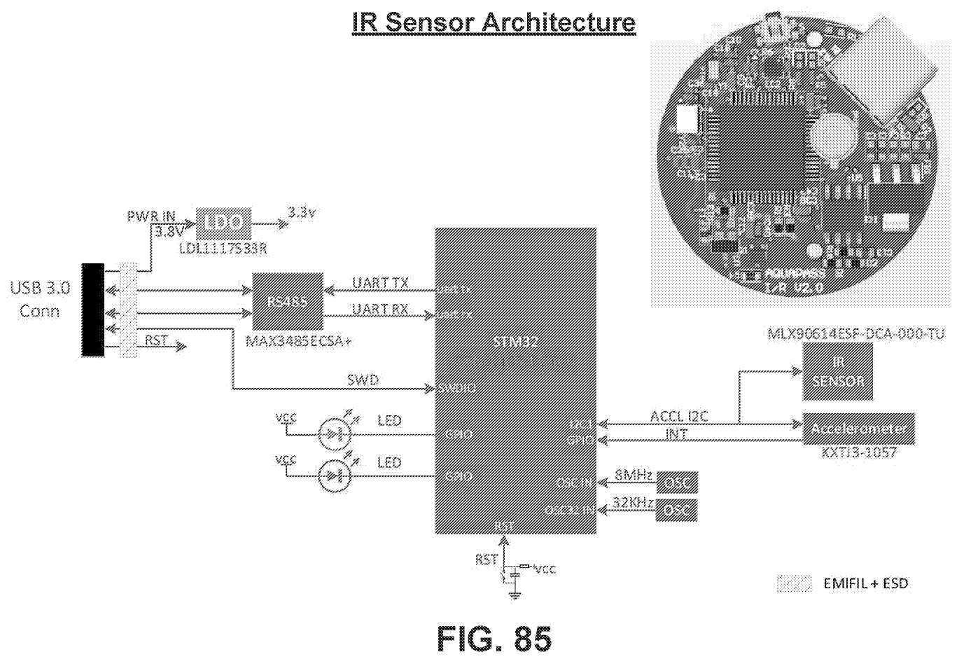

FIG. 85 is a circuit diagram representing one embodiment of an IR sensor architecture.

As described herein, the controller of the console unit processes temperature and relative humidity measurements collected via sensors at the inlet and outlet ports of the treatment device and, together with airflow measurements collected via a flow sensor the console unit, is able to continuously calculate a sweat rate of the patient during the procedure. During a procedure it is assumed that if sweating occurs, the air exiting the treatment device will be more humid than the air entering the treatment device. Accordingly, the amount of fluid removal depends on the sweat rate. The fluid removal rate is measured by calculating the difference between the patient weight before and after procedure. Fluid removal rate can also be estimated using the following equation *:

$$Air Flow rate ( \frac{m 3}{min} ) \times Absolute humidity inflow outflow diff ( \frac{g}{m 3} ) = Sweat rate ( \frac{g}{min} )$$

*Pressure throughout the wearable is maintained and therefore can be neglected.

Depending on the given treatment, including the specific parameters of the treatment (i.e., planned fluid removal amount and overall time for procedure), the controller 110 is able to control output from the console unit 106 on-the-fly based on sweat weight calculations, including adjusting flow rate and/or inflow air temperature to the treatment device until a sufficient sweat rate is achieved.

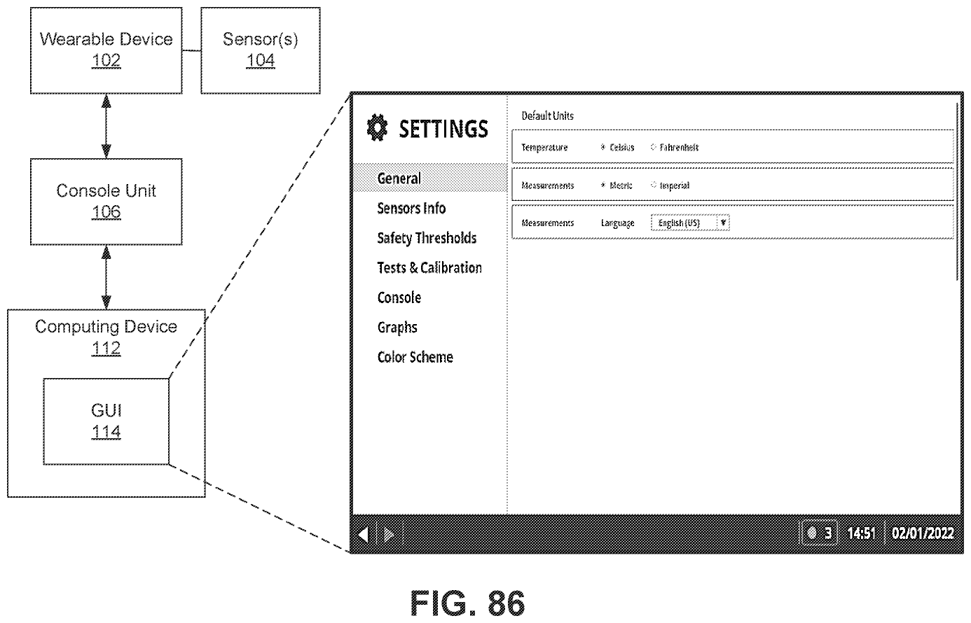

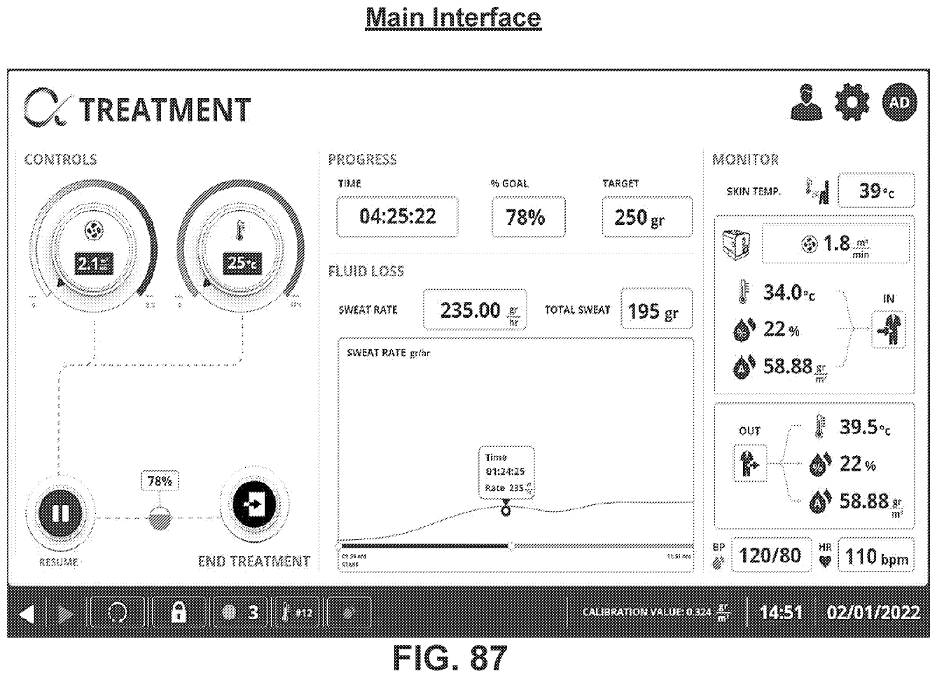

FIG. 86 is a block diagram illustrating communication of data between the wearable treatment device, sensors, console unit, and computing device, including an initial settings screen provided via a graphical user interface (GUI) of the computing device. FIG. 87 is an exemplary screenshot of a user interface providing various information related to the fluid stimulation system, including details of a given procedure.

As shown in FIGS. 86 and 87, the console unit, including the controller, may generally be controlled via a software-based application running either as a web-based app or running as a local app downloaded on a user's computing device 112. For example, a user (i.e., physician or other medical professional) may utilize a personal computing device 112 (e.g., laptop, tablet, smartphone, etc.) to access software associated with the fluid stimulation system, in which a graphical user interface (GUI) 114 is provided allowing for the monitoring of a given procedure, as well as subsequent control of such procedure. The computing device 112 is configured to communicate and exchange data with the console unit and controller via a wired or wireless connection (i.e., Wi-Fi network, Bluetooth radio, Near Field Communication (NFC), etc.). The computing device 112 may also be configured to communicate with other sensors not incorporated into either of the treatment device or console unit, including sensors for monitoring patient vital signs (i.e., heart rate, blood pressure, saturation, etc.). For example, in some embodiments, the system 100 further comprises a blood pressure monitor, heartbeat monitor, and core body gauge to monitor the patient parameters during the treatment. The system stimulates fluid loss without changing the patient blood pressure, core temperature, or heartbeat. In certain embodiments of the invention, the chamber is fixed to a surface, such as a bed or chair. In other embodiments, the chamber is mobile or wearable by a patient.

As shown in FIG. 86, the GUI may present a settings function, in which various aspects of the procedure, and presentation of data via the interface, can be changed to a user's preferences. The ability to customize settings may be based on role-based authorization schemes, in which, depending on a user's role within a given organization, a user may have greater or less access to customizing certain settings as a result of their role. For example, administrators may have greater access than operators, while technicians and developers have greater access than administrators. The authorization level is determinative as to which fields within the settings function will be editable, read-only, or not visible at all to the given user.

FIG. 87 illustrates an exemplary screenshot of a user interface providing various information related to the fluid stimulation system, including details of a given procedure. For example, the user interface provided on a user's computing device may provide real-time data during the procedure, including, but not limited to, information associated with a current treatment procedure (i.e., elapsed time since treatment began, sensor measurement data and related calculations of the environment provided by the treatment device, including air temperature and absolute humidity, skin temperature, current sweat rate, accumulative sweat measurement, and more).

In certain embodiments, the system comprises a body scale to measure the body weight of the patient. In some embodiments, the device measures fluid loss during the treatment by measuring the patient body weight before, during, and after the treatment. One or more temperature sensors or sweat sensors might be placed on the patient body outside of the chamber, such as on a face or a neck of the patient, in order to provide a reference measure of sweat and skin temperature during treatment. In some embodiments, the system comprises a clock to measure the duration of the treatment. In some embodiments, the system further comprises a skin temperature sensor. The skin temperature sensor is placed on the skin of the patient and monitors the skin temperature and verifies that the skin temperature does not, in any circumstance, elevate above 39° C. If the skin temperature elevates above 39° C., the system is configured to stop operation of the device.

Furthermore, the user interface may provide a user with input controls for controlling the given treatment, including basic start and stop controls, controls for selecting as well as control over output from the console unit (i.e., air flow rate and heat output).

For example, as provided in Table 1 below, the user interface provides a various input controls and information to a user.

TABLE 1
User Interface Control Panel

| Indication | Related Description |
|---|---|
| Start | Start Blower and Heaters |
| Pause | Stop Blower and Heaters, Stop Writing to log. |
| Resume | Start Blower and Heaters, Continue Writing to log. |
| Restart | Zero all treatment data and clear log |
| Summary | Go to summary screen |
| Temperature | Adjust desired temperature |
| Input | |
| Blower Input | Adjust desired flow rate |
| End Treatment | Stop Blower and Heaters, Stop Writing to log, Send log by email and/or |
| | save to a flash drive and/or locally |
| Core | Sampled and input into system manually |
| Temperature | |
| Blood Pressure | Sampled every 30 mins with possibility for manually sampling |
| and Heart Rate | |

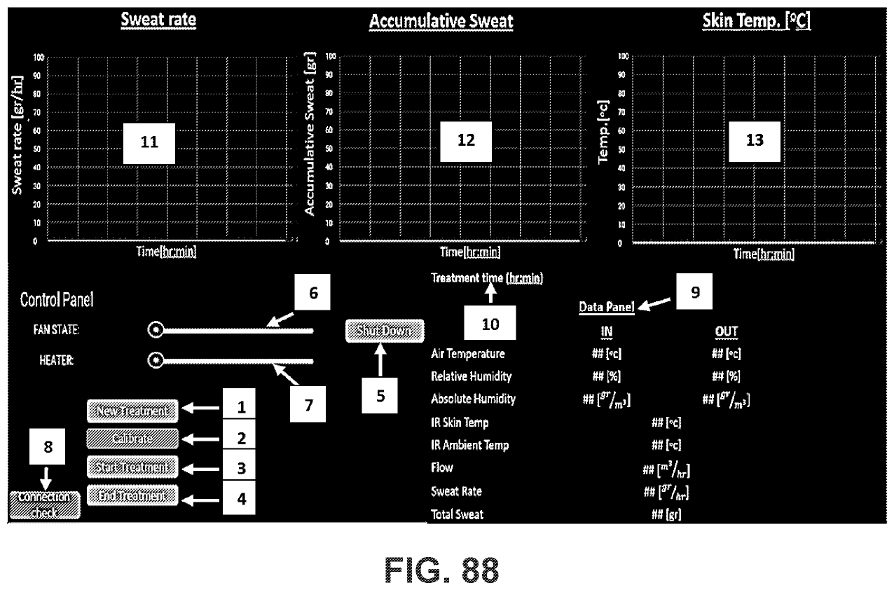

FIG. 88 illustrates another exemplary screenshot of a user interface providing various information related to the fluid stimulation system, including details of a given procedure. For example, as provided in Table 2 below, the user interface provides a various input controls and information to a user.

TABLE 2
User Interface Control Panel

| Indication | Related Description |
|---|---|
| 1 | “New treatment” button |
| | This button opens a new log file. |
| | Note: New treatment shall be initiated before the patient enters the Wearable. |
| 2 | “Calibrate” button |
| | This button starts a 1 min measurement of the sweat-rate and calibrates the |
| | inlet and outlet sensors against each other. |
| 3 | “Start treatment” button |
| | This button starts the treatment and starts counting the treatment time and |
| | calculation of the total sweat-rate. |
| 4 | “End treatment” button |
| | This button ends the treatment and closes the log file. |
| 5 | “Shut down” button |
| | This button shuts down the heater and the Blower |
| | Note: The shut down procedure occurs also when overheating is detected |
| 6 | Blower slider |
| | This slider controls the Blower level (flow-rate in the system) |
| 7 | Heater slider |
| | This slider controls the heaters level (the heat in the system) |
| | Note: This slider is limited to 50% |
| 8 | Bluetooth connection check |
| | This button is an indication of the Bluetooth connection (disabled). Green - |
| | connected, red - disconnected |
| 9 | Data panel |
| | This panel presents the sensors' measurements and related calculations of the |
| | absolute humidity and sweat-rate. |
| 10 | Treatment time indicator |
| | The time presents the treatment time after the “start treatment” button was |
| | pressed. |
| 11 | Sweat-rate graph |
| | This graph presents the sweat-rate during the treatment. |
| 12 | Accumulate Sweat graph |
| | This graph presents the total sweat during the treatment. |
| 13 | Skin temp. graph |
| | This graph presents the skin temperature during the treatment. |

Accordingly, the system of the present may include a treatment device and a console unit, including input sensors and meters, which may include any one or more of:
1. A skin temperature sensor placed on a part of the body that its temperature is being raised. It can be on the leg, abdomen, torso or back;
2. Two relative humidity and temperature sensors, placed one at the warm air inflow line and the second at the warm air outflow port. Online readings may be sent to the control unit that then calculates the absolute humidity form the relative humidity and temperature;
3. Air flow meter placed in the inflow line measuring continuously the air flow rate into the wearable. The online readings may be sent to the control unit;
4. A weight scale to measure the patient's weight before the procedure and at the end of the procedure. The weight sends the recording to the control unit;
5. A warm air heater that is set to deliver air at temperatures between about 37 degrees Celsius and 55 degrees Celsius and at relative humidity of up to 40% at 40 degrees Celsius; and

6. A control unit that receives input readings from the above sensors and meters calculates the sweat rates by subtracting the inlet from the outlet absolute humidity and then multiply by the air flow rate. The calculated sweat rate is then evaluated relative to the required sweat rate and the required weight loss.

In instances where the sweat rate is too low or too high or the relative humidity elevates above, for example 60%, the control unit may set the inflow air temperature and flow rate to meet the desired sweat rate and weight loss that's required per treatment and reduce the relative humidity to enable immediate evaporation. The control unit may also send the information and treatment parameters by the internet to the treating nurse or physician.

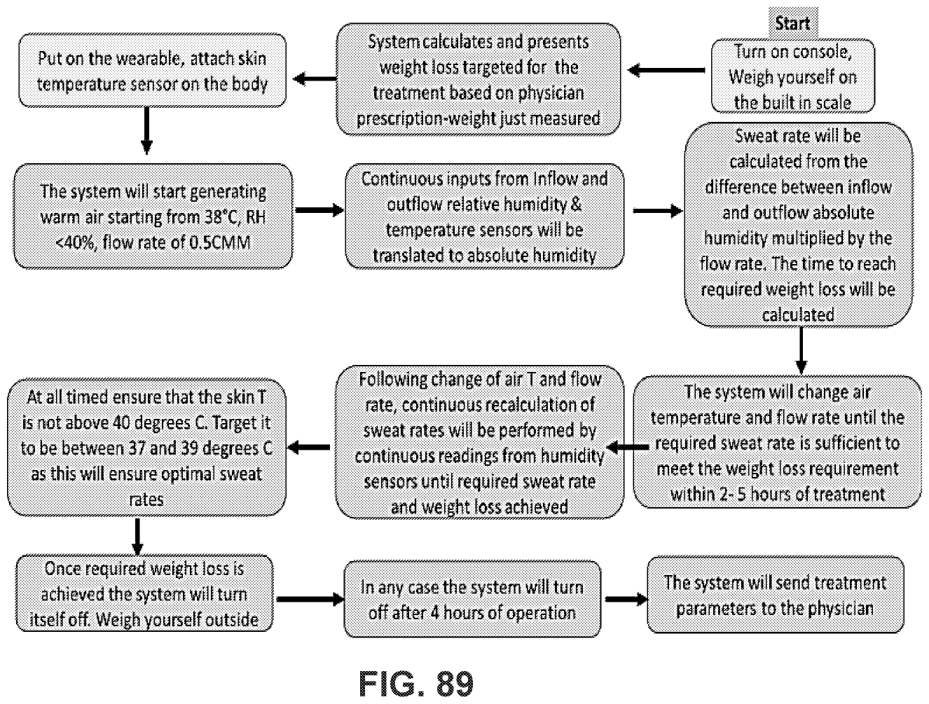

An algorithm executed by the control unit is presented in FIG. 89. The algorithm ensures that a treatment is set automatically with maximal safety and is thus suitable to, for example, an elderly patient that cannot operate devices that require complicated operation and thinking. For example:
The patient is told that his target weight should be 80 kg.
The patient turns on the device and the target weight is being set in the set up screen or sent by the physician by the home wireless internet.
He weighs himself on the integrated system weight and he is 81 kg.
The system generates a signal or a voice telling the patient that he will be treated for 3-5 hours depending on the sweat rate.
The patient wears the wearable and the system automatically starts to send in warm air at 40° C. and relative humidity per the ambient air but less than 40% and at a flow rate of 0.5 cubic meter/minute (CMM)
The system measures the relative humidity and temperature at the outlet and inlet ports and calculates the sweat rate. For example, the calculated sweat rate is 150 ml/hr and the relative humidity is 75%. The system will increase the flow rate to reduce the humidity to less than 60% (to enable full evaporation, more efficient sweating and maintaining core temperature at normal levels).
If the skin temperature is below 37° C. the system will also elevate the input temperature until the skin T is above 38° C. but less than 40° C.
Once the system detects a sweat rate of 200 lml/hr and skin T between 37-39 degrees and relative humidity below 60% then the system will set these parameters and determine the treatment duration.
Once the calculated targeted weight is met the system stops the operation and the patient is asked to weigh again.

### Additional Features of the System

1. The outlet air will be connected to the inlet of air to the console. This will enable reduction in power consumption as the air entering the console will be already warm and will require only small additional warming up. There will be a condenser at the inlet to dry the humid air.
2. The console will have an integrated blood pressure, HR and saturation monitoring devices enabling timely checkups and data logging.
   The software will initiate the treatment only after initial BP, HR and saturation has been measured and request the patient to re measure the parameters once again after 30 minutes of treatment and if all parameters are acceptable then every 1-2 hours.
   The console will have an integrated multifunction weight connected to it and reading its values. It will serve as additional information displayed to the patient and physician. The weight will have the ability to differentiate between weights that are from body fluids or from non fluid body compartments such as bone, muscles and other connective tissue.
   In addition to the system the patient will have a sweat electrolyte analyzer that will analyze the results and recommend the patient if and how much to drink.
   For example if the total sweat secreted is 1 Liter and the sweat sodium concentration will be below 40mmol/L of sweat the patient will be advised to drink 300 ml of fresh water during the procedure so as not to elevate the sodium concentration.

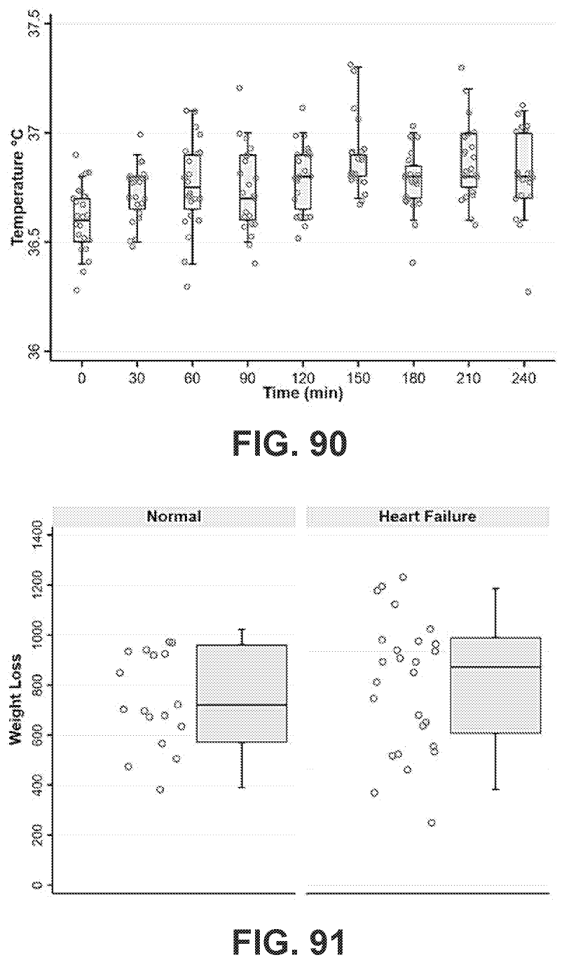

FIGS. 90, 91, 92, 93, and 94A-94B show clinical data from using systems and methods of the invention, as will be described in greater detail herein.

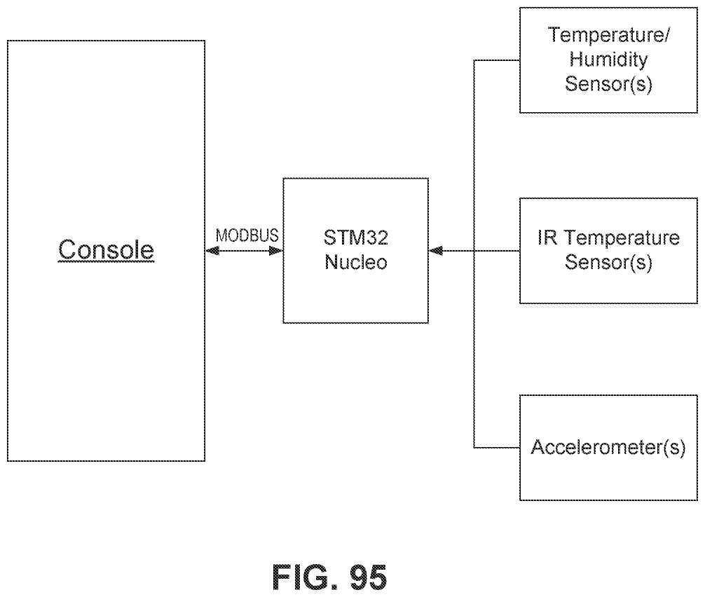

FIG. 95 is a block diagram illustrating communication of data from the various sensors to the console unit via a microcontroller (a STM32 Nucleo development board). As illustrated, the console unit may receive feedback data collected from the temperature/humidity sensors (associated with the inlet and outlet ports of the wearable device), the IR temperature sensor, and the accelerometer via associated microcontrollers (STM32 microcontroller).

Accordingly, in one embodiment, the wearable device includes at least three temperature/humidity sensors (a sensor associated with the inlet port and the two outlet ports) coupled to an associated microcontroller. Sensor data is measured and updated about every 0.2 seconds, and the temperature and/or humidity measurement is averaged between the three sensors. For example, temperature and/or humidity data is communicated to the console unit, wherein the temperature and/or humidity data is an average of the temperatures and/or humidity measured from each of the temperature/humidity sensors. If one of the sensors measures a temperature and/or humidity beyond two standard deviations of the other two temperature and/or humidity measurements, then an error measure is sent indicating a discrepancy.

In particular, temperature data corresponds to two bytes of data, such that an obtained value undergoes the following function:

$${T}_{C e lcius} = \frac{{T}_{m easured} * 17500}{65535} - 4500$$

wherein the result is temperature in Celsius.

Humidity data corresponds to two bytes of data, such that an obtained value undergoes the following function:

$${Hum}_{percent} = \frac{{Hum}_{measured} * 10000}{65535} - 4500$$

wherein the result is percentage of humidity in air.

Similarly, a patient's skin temperature data is obtained as two bytes of data, and converted to a floating point value, such that the data undergoes the following function:

$${T}_{Celcius} = {T}_{measured} * 0.002 - 273 / 15$$

wherein the result is temperature in Celsius.

As previously described, the system may also include one or more accelerometers relative and in proximity to the patient's body for monitoring patient movement, specifically collecting acceleration data of the patient and used in determining a patient's pose (i.e., a supine or prone position, a sitting position, or a standing position). For example, acceleration acting on sensors is obtained as two bytes of data per axis, and converted to floating point values, such that obtained data is divided by 15987, wherein the result is acceleration in terms of Earth's gravity. In order to estimate a patient's particular pose, a calibration step must first be performed. The calibration step involves having a patient remain still for a set period of time (e.g., 5 seconds). During this time, measured acceleration corresponds to Earth's gravity, such that a global reference frame is calculated. In order to avoid drift, when user's acceleration is below a threshold for a certain amount of time, data from a portion of that interval is used to recalibrate. To determine a state of transition from one pose to another, a patient's acceleration data is integrated over a period of time (e.g., 10 seconds). When this value is above a threshold, it indicates a transition from sitting to standing and when the value is below a threshold, it indicates a transition from standing to sitting or lying down. When the user is lying down, that state can be identified due to Earth's gravity acting on a 90-degree angle in relation to sitting and standing.

As described throughout, the wearable treatment device of the present invention is able to provide a warm air environment at a controlled air volume flow rate around a body part of a patient to shift fluids directly and non-invasively from an interstitial compartment of the patient to skin of the patient resulting in controlled fluid loss. The console unit is able to control a given treatment procedure based, at least in part, on feedback data received from various sensors. In particular, the console unit may include a controller communicatively coupled with a plurality of sensors that provide data to the controller for calculating at least a sweat rate caused as a result of the warm air environment provided by the treatment device, the controller comprising a processor coupled to memory containing instructions executable by the processor to cause the controller to control and monitor conditions of the treatment device. More specifically, the controller is configured to adjust output of at least one of air and heated air from the console unit to thereby control at least one of relative humidity, airflow, pressure, and temperature within the treatment device based on feedback data received from the plurality of sensors.

In order to improve responsiveness, decrease temperature variance during operation, improve stability and user comfort, the system of the present disclosure may further include a proportional-integral-derivative (PID) controller to control the heater element of the console unit. Unlike an on/off controller, a PID controller allows a system to maintain a constant temperature throughout long operation periods, and is quicker to respond to changes in the environment. In some embodiments, the system may include an automatic PID adjuster, in order to account for possible differences between heaters of the console unit.

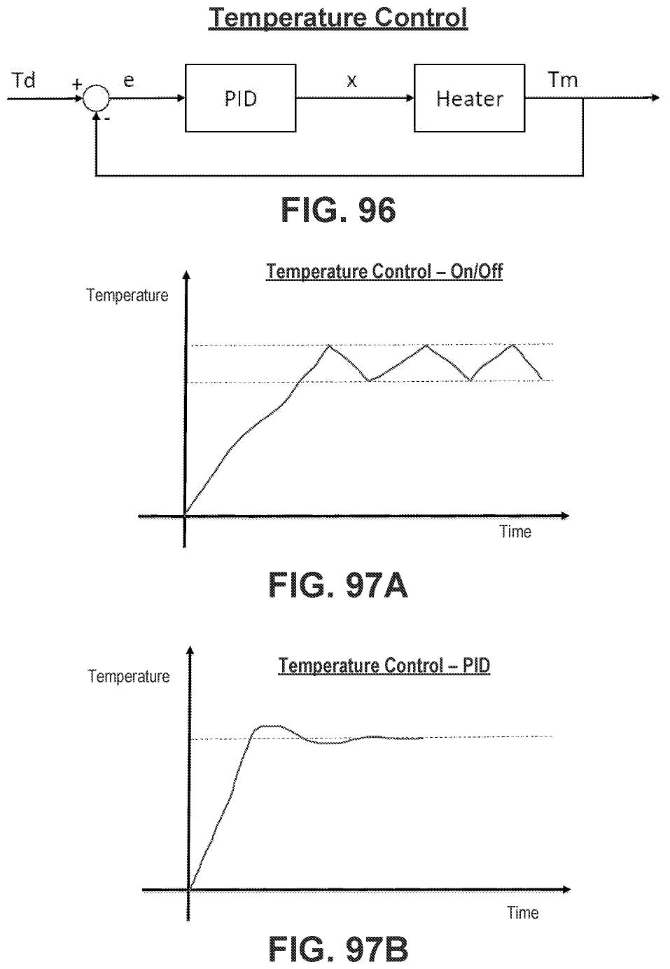

FIG. 96 is a block diagram illustrating one embodiment of controlling the temperature output of air (to be supplied to the wearable treatment device) via a proportional-integral-derivative (PID) controller, wherein Td is the desired temperature, Tm is the measured temperature, e is an error, x is a signal sent to the heater, such that e=Td−Tm. The PID controller may operate in accordance with the following function:

$$x ( t ) = Kp * e + Kd * \frac{e ( t )}{clk} + Ki * {\sum }_{n = 0}^{t} e ( n )$$

where Kp, Kd, and Ki are constants determined based on system behavior.

FIGS. 97A and 97B are graphs illustrating temperature control via a conventional on/off operation and a via PID control consistent with the present disclosure, respectively.

Accordingly, the system of the present may include a treatment device and a console unit, including input sensors and meters, which may include any one or more of:
1. A skin temperature sensor placed on a part of the body that its temperature is being raised. It can be on the leg, abdomen, torso or back;
2. Two relative humidity and temperature sensors, placed one at the warm air inflow line and the second at the warm air outflow port. Online readings may be sent to the control unit that then calculates the absolute humidity form the relative humidity and temperature;
3. Air flow meter placed in the inflow line measuring continuously the air flow rate into the wearable. The online readings may be sent to the control unit;
4. A warm air heater that is set to deliver air at temperatures between about 37 degrees Celsius and 55 degrees Celsius and at relative humidity of up to 40% at 40 degrees Celsius; and
5. A control unit that receives input readings from the above sensors and meters calculates the sweat rates by subtracting the inlet from the outlet absolute humidity and then multiply by the air flow rate. The calculated sweat rate is then evaluated relative to the required sweat rate and the required weight loss.

In instances where the sweat rate is too low or too high or the relative humidity elevates above, for example 60%, the control unit may set the inflow air temperature and flow rate to meet the desired sweat rate and weight loss that's required per treatment and reduce the relative humidity to enable immediate evaporation. The control unit may also send the information and treatment parameters by the internet to the treating nurse or physician.

As described throughout, the treatment devices of the present invention are able to provide a warm air environment at a controlled air volume flow rate around a body part of a patient to shift fluids directly and non-invasively from an interstitial compartment of the patient to skin of the patient resulting in controlled fluid loss. The console unit, which can be connected to the device (as well as the various sensors) via a wired or wireless connection, is able to control a given treatment procedure based, at least in part, on feedback data received from various sensors. In particular, the console unit may include a controller communicatively coupled with a plurality of sensors that provide data to the controller for calculating at least a sweat rate caused as a result of the warm air environment provided by the treatment device, the controller comprising a processor coupled to memory containing instructions executable by the processor to cause the controller to control and monitor conditions of the treatment device. More specifically, the controller is configured to adjust output of at least one of air and heated air from the console unit to thereby control at least one of relative humidity, airflow, pressure, and temperature within the treatment device based on feedback data received from the plurality of sensors.

For example, the console unit may be configured to adjust output of at least one of air and heated air therefrom based, at least in part, on an AI-based algorithm. More specifically, the console unit may be configured to obtain and process feedback data received from the plurality of sensors, wherein the feedback data comprises at least one of skin temperature, ambient temperature, core patient temperature, blood pressure, heart rate, sweat rate at the ambient temperature and sweat rate at the skin temperature. The feedback data may be processed using a machine learning system. For example, the machine learning system may include a neural network trained to identify optimal working parameters of the system with respect to a given patient undergoing treatment for fluid stimulation. The controller is configured to adjust output of air and/or heated air therefrom based on the identified optimal working parameters so as to achieve an optimal warm air environment around the body part of the patient. The processing of the feedback data comprises correlating the feedback data with reference data. The reference data comprises prior treatment data of the patient, such as the history of treatments, prior working parameters and resulting sweat rate as a result of such working parameters, and the like. The machine learning system is selected from the group consisting of a random forest, a support vector machine, a Bayesian classifier, and a neural network.

It should be noted, in some instances, a patient may be provided with a medicament to assist in further controlling a sweat rate during a given procedure. For example, it has been found that sweat rate can be increased by reducing a patient's core temperature set point of the hypothalamus. In particular, by reducing the temperature set point of the patient, the gradient between the ambient temperature and the core temperature will be greater, thereby increasing rate. Reducing the patient's core temperature setpoint can be a much safer option in achieving the greater gradient, as opposed to increasing the ambient temperature, as this would result in skin temperatures that are too high and would risk burns or injury. In order to reduce the set point core temperature, antipyretic drugs, such as Ibuprofen, can be given to the patient. Such drugs will reduce the core temperature by around 0.5 degrees Celsius. Additionally, or alternatively, treatment can be performed while the patient is asleep (i.e., sleep reduces the core temperature by 1 degree Celsius.

Accordingly, sweat rate can be further enhanced during a treatment procedure provided by systems of the present invention by reducing the set point core temperature of the patient's hypothalamus.

### EXAMPLES

### Methods and Devices to Remove Extracellular Fluid Overload (Edema) via the Skin

Extracellular Fluid Overload (Edema) is a medical condition in which excessive fluid accumulation in the intravascular and/or interstitial compartments occur. If left untreated tissue function as well as gas exchange can be compromised leading to hospitalization.

The causes of the edema can be of many etiologies some of which are elevated intravascular hydrostatic pressures, changes in either intravascular or interstitial osmotic pressure, lymphedema, hypertension and obesity.

Accordingly, the invention recognizes that methods and devices that reduce edema whilst the patient is still in a chronic state and not acutely hospitalized could be very beneficial.

In some aspects, the present invention provides systems and methods for stimulating fluid transfer through the skin in a subject for treating chronic edematous clinical conditions in the subject and before they become acute heart failure episodes.

For example, fluid from the extracellular compartment can be removed directly from the skin by either sweat production in the sweat glands or via the dermis by means of osmosis.

It is generally accepted that fluid removal rates ranging from 50 ml/hr-300 ml/hr can enable prevention of excessive edema that if left untreated can lead to acute decompensation and hospitalization.

Fluid can flow out of the skin through the sweat glands at a rate of liters per hour if the sweat glands are stimulated in the most aggressive way. However, may not be tolerated by patients and may result in a hemodynamic shock. The method may locally elevate a temperature of a patient's skin to values from 35° C. to 38.5° C. by applying warm (38° C.-44° C.) and low humidity (less than 40%) air environment around the patient's lower half of the body for durations of 1 hr-6 hr. The timing may depend on factors such as edema severity. Such a method may achieve a fluid removal rate of between 100 ml/hr-300 ml/hr.

Air may be supplied by a generator console which has control of air temperature, flow rate, humidity and additional parameters which assure safe and effective operation. The lower part of the body is the preferred area of treatment as it will be better tolerated by the patient; however, the invention recognizes that other parts of the body may be treated, such as, the hands, chest, or back, especially if those other parts are edematous.

Partial body skin temperature elevation to values between 35° V and 38.5° C. are expected to maintain core body temperature at normal values elevating no more than 0.5° C. at maximum and initiate sweat at a rate that between 100 ml/hr-300 ml/hr when the part of the body that its skin pressure is elevated is the lower prat of the body from the abdomen and lower back down to the feet.

In some respects, part of the method may relate to treating a patient either outside the hospital, i.e., in his home environment, or in the outpatient clinics, so as to prevent acute medical conditions or hospitalization.

For the patient to tolerate daily or weekly 1-6 hr treatments, it is desirable that the device enables the treated patient to be mobile whilst being treated. Therefore, the console unit preferably enables elevation of the skin temperature to values between 35° C. and 38.5° C. while the patient can be mobile. As such, the patient may be mobile whilst being treated. A specific apparatus may be worn by the patient, along with a specific heat generator, to enable mobility.

### Methods and Devices to Control Extracellular Electrolytes and Fluid Balance via the Skin

Extracellular electrolytes balance, especially sodium and potassium concentrations in the interstitial compartment are important to preserve hemostasis and prevent water retention. Ways to control the electrolyte concentration may include food and fluids intake or by drugs that work on the kidneys to either dispose of or preserve electrolyte concentration.

In many pathologies, such as chronic kidney dysfunction (CKD) or heart failure (HF) there is an electrolyte and fluid imbalance that leads to hospitalization or chronic need for dialysis.

Switching to another biological system, sweat glands secrete fluids from the interstitial compartment. The fluids that enter the secretory coil part of the sweat gland is isotonic to plasma with Na+ concentrations of around 140 mmol/liter.

Once sweat flows towards the skin surface part of the sodium gets reabsorbed by the Apical and Basolateral membranes of the sweat duct according to the leak-pump model. Because of the reabsorption the Na+ concentration on the skin is hypotonic and can range, depending on the sweat rate between 10 mmol/Liter-100 mmol/Liter. Controlling the sweat sodium concentration can have a great effect on the fluid status of a patient with CKD or HF and potentially prevent them from being fluid overloaded.

The plasma Na+ concentration is strictly controlled between a small range of less than 10mmol/Liter and therefore alterations from this range to either hypo or hypernatremia can have grave consequences.

In some aspects, this disclosure relates to methods and systems for controlling fluid and electrolytes such as sodium, potassium and chloride concentration by controlling their transfer through the skin during sweat.

Applying a warm air environment around a part of the body may initiate sweat when the skin temperature elevates above 33° C. To ensure that the skin does not start to burn, the temperature of the skin is preferably always be kept below 39° C.

Methods disclosed herein may control the reabsorption rate of the sodium ions at the duct by attracting them to the skin surface using a negatively charged ion at specific relative humidity environment produced by the apparatus on the skin surface or in the air surrounding the skin.

For example, if surrounding the skin, whilst sweating is stimulated, would be a negatively charged carbonate CO₃ or OH anion, the sodium cations in the duct may enhance the flow towards the skin rather than being diffusing into the apical membrane. The reabsorption rate of the sodium into the interstitial compartment of the skin will be reduced and the sodium concertation will not elevate. This clinically could be very meaningful as the electrolytes, especially the sodium, determines edema status by binding to fluids.

In some aspects, this disclosure provides a combination of stimulating sweat production and controlling the electrolytes reabsorption rate, thus increasing or decreasing the extravascular sodium and potassium or any other electrolyte concentration.

One example of an apparatus of the invention includes a single, or multiple use, 2 stage apparatus that is placed in the air inflow line of a warm air generator to increase the skin temperature to initiate sweating. In the first stage of the apparatus, a soluble salt such as K₂CO₃ may be dispersed into the inflow of air at a temperature of, for example, 40° C.-46° C. and at a relative humidity of between 30%-50%.

The salt may be soluble and may naturally separate into cations and anions.

In the second part of the apparatus, the air with the K₂+CO₃-ions may be passed through a cation resin filter beads that filter the cations and only allow for the anion in the air (H+ and K₂+) to flow onto the skin inside the apparatus.

The air, being negatively charged, may increase sodium attraction to the skin surface from the ion bonding attraction and as a result, decrease the reabsorption rate of the sodium. Preferably, the sodium concentration in the interstitial compartment will decrease in addition with plasma sodium levels.

Another apparatus may be a filter. The filter may be placed inline to the air inflow line in which all the current electrolytes in the air are filtered. An environment of humidity of around 50% may be created by passing the air into a hydrated environment. As a result, the capacity of the air to attract the sodium cations may increase as they will bind to the negative ions of the humid air water molecules.

Other methods for attracting the sodium molecules to the skin surface may involve using a negative hydrostatic pressure environment while stimulating sweat. The negative hydrostatic pressure may enhance sweat flow to the skin and increase the sodium concentration on the skin and reduce its interstitial concentration.

An exemplary apparatus may be a permeable container loaded into the inflow of the air and having K₂CO₃ salt that has a solubility in moist air that is very high. Once partially dissolved, CO₃ anions will then attract the Na+ electrolytes from the sweat duct and will reduce their reabsorption.

Accordingly, in some aspects, this disclosure relates to a method to reduce sodium concentrations in the plasma and interstitial compartment by enhancing sweat rate and pulling to the skin surface the sodium cations using air having negatively charged anions.

It should further be noted that patients with heart failure suffer from frequent fluid overload in the interstitial and intravascular compartments as a result of elevated hydrostatic or osmotic or both these pressures within the capillaries. The elevated pressures lead to fluid overload formation. This is a serious medical condition and can become a life-threatening.

Current therapies mostly rely on drugs that reduce intravascular volume and pressure by enhancing urine production. These therapies require complicated sequence of clinical events and often lead to kidney dysfunction, drug resistance and inefficient treatments. New therapies are highly required to meet this great unmet clinical need that millions of patients suffer from.

The fluids in the interstitial compartment supply the sweat fluids to the eccrine glands and therefore can be secreted out of the body through a natural pathway of these glands through the skin. This natural pathway can be used to decongest fluid overload and treat HF patients. In order to efficiently decongest these patients suffering from fluid overload through the skin a sweat rate of between 100 ml/hr-500 ml/hr is recommended. Lower rates may not be efficient and too high rates can be dangerous and lead to hypotension.

It is highly recommended to have an option to treat the patients also at home and therefore a controlled and automated system that will decongest the patient's fluid overload without the requirement of in-hospital stay nor constant monitoring can be beneficial.

There are currently no methods nor devices to treat patient fluid overload by enhancing fluid decongestion through the skin.

There are other several methods by which to promote fluid transfer through the skin. For example: Elevation of core temperature, however, it may be difficult for HF patients that will not tolerate the increase in core T heat; neural stimulation by drugs, although may not be tolerable for long durations; elevation of skin temperature, which is a preferred method by which to promote perspiration and be tolerable and even comfortable for the patients if subjected only to the lower part of the body from the chest down.

The skin starts to sweat at skin temperatures of usually over 33 degrees Celsius, but varies from one person to the other.

Accordingly, in some aspects, this disclosure provides both a method and apparatus for decongesting fluid overloaded patients by elevating and controlling partial body skin temperature to values over 36 degrees Celsius and typically between 37-39 degrees Celsius. These skin temperatures may enhance fluid flow from the edematous regions in the interstitial compartment to the eccrine and/or apocrine sweat glands and out to the skin. Immediate evaporation of the sweat on the skin may be accomplished by the low humidity and continuous airflow that elevated the skin temperature. The evaporation process cools down also the blood flowing to the skin and upon its return to the deeper vasculature it ensures that the core body temperature remains within normal values. This effective treatment can be performed whilst maintaining core temperature values, HR and BP at baseline/normal levels.

In some aspects, this disclosure describes an apparatus that elevates partial body skin temperature by covering part of the patient with a loose and self-wearable fabric that is worn around the chest and upper back all the way down to the feet, preferably, including the toes. A tighter elastic band of up to about 5 cm wide or a tightening string, may be placed around the chest and lower back of the wearable fabric, and may be built in so as to eliminate any air flow in nor out towards the face and head from that region.

The fabric may be made form a mesh, for example. The mesh ensures flow of air to every region of the body thus enabling sweating and evaporation. Due to the mesh structure even when the patient is seated down, the patient may touch the wearable just at a very small region whilst around it the air still flows, elevates the skin temperature and evaporates the sweat when it flows out.

Preferably, there is an air inlet port and an air outlet port at opposite sides of the wearable. The inlet may be at the feet and the outlet may be at a level of the chest, or visa versa. Warm air from a heater may be directed into the wearable from the inlet port. The air can be diverted using smaller tubes to all parts of the body. The warm air may heat up the skin temperature until it initiates sweating. The air may leave the wearable at higher humidity then its inlet humidity as it now has the sweat that evaporated from the skin in it. The air may evaporate the sweat very quickly as the relative humidity will always be kept below, for example, 40% at the inlet ensuring that also with maximal sweat rates at the outflow the humidity will not exceed, for example, 60%.

The warm air may initiate sweat when the skin temperature elevates above a certain temperature that varies slightly between one person to the other but in general may be above 32 degrees Celsius and below 36 degrees. Above this onset temperature the sweat rate may increases as the temperature elevates. To ensure that the skin does not start to burn, the temperature of the skin may be kept below 42 degrees Celsius.

### Methods and Devices to Treat Pre-Dialysis via the Skin

In the United States, more than 114,000 individuals with end-stage renal disease (ESRD) initiate maintenance dialysis each year. The prevalence of ESRD more than tripled between 1990 and 2018. Globally, the number of patients undergoing maintenance dialysis is increasing, yet throughout the world there is significant variability in the practice of initiating dialysis. As ESRD is a rapidly increasing global health and health care burden, the inability to care for many patients at risk for and in need of treatment for ESRD disproportionately impacts low-and middle-income countries.

The optimal time to commence maintenance dialysis in patients with chronic kidney disease has been recognized by nephrologists internationally as one of the most important dialysis-related question to be addressed. The IDEAL study results and the growing body of observational data suggesting no benefit to early initiation of dialysis. In the IDEAL trial involving patients with stage V chronic kidney disease, early initiation of dialysis had no significant effect on the rate of death from any cause or on cardiovascular events, infectious events, or complications of dialysis.

The most recent iteration of the KDOQI guidelines for hemodialysis adequacy, published in 2015, even more explicitly avoid a focus on eGFR and recommend that the decision to initiation maintenance dialysis should be based primarily on assessment of specific complications of kidney disease, including signs and symptoms of uremia, protein-energy wasting, metabolic abnormalities, and volume overload, rather than based on a specific level of kidney function.

Specifically, initiation of dialysis is usually considered when one or more of the following are present:
1) symptoms or signs attributable to kidney failure (e.g., neurological signs and symptoms attributable to uremia, pericarditis, anorexia, medically resistant acid-base or electrolyte abnormalities, reduced energy level, weight loss with no other potential explanation, intractable pruritus, or bleeding).
2) Inability to control volume status or blood pressure.
3) A progressive deterioration in nutritional status refractory to interventions.

Comprehensive conservative care is defined as ‘planned holistic patient-centered care for patients with stage 5 CKD and including a full range of treatment and support, but not dialysis’. When considering the options of conservative management and dialysis, decision-making can be difficult and there are no randomized studies comparing outcomes between patients choosing conservative management and dialysis. Data from observational studies suggest comparable survival in older patients and those with significant comorbidities or poor performance status.

In addition to survival, the influence of treatment modality choice on other factors such as measures of quality of life, the number of hospital-free days, symptom burden, travel and the effect on family should be considered.

Initiating chronic dialysis has major implications for patients and health-care systems. Early initiation of dialysis also exposes the patient to complications of dialysis, unnecessary lifestyle restriction, and potential increased costs. Thus, the optimal timing of initiation of dialysis is unclear.

Dialysis reduces the patient's quality of life and is best delayed until the optimum point. Depending on the patient's preferences and circumstances, an aggressive trial of medical nondialytic management of advanced CKD symptoms may be warranted before initiating maintenance dialysis. The Canadian Society of Nephrology recommends an “intent-to-defer” over an “intent-to-start-early” approach for the initiation of chronic dialysis. However, there is no guidance on how the pre-dialysis period can be safely prolonged.

Considering the enormous burden imposed by dialysis on patients and health care systems, there is an unmet need for prolonging the pre-dialysis period in patients with residual renal function.

Enhancing eccrine sweat glands activity can represent an additional route for sodium chloride, potassium, urea, and fluid removal in pre-dialysis state, that is not affected by renal dysfunction. Other electrolytes and metabolites excreted in sweat includes ammonia, magnesium, and phosphate. This suggests that enhancing sweat rate using the systems and methods of the present invention can allow safe prolongation of the pre-dialysis phase and benefit patients under conservative management.

Furthermore, it is now understood that the composition of sweat is such that the urea content is much higher than in serum. Accordingly, systems and methods of the present invention can be used to potentially treat pre-dialysis patients, in addition to actual dialysis patients. For example, systems of the present invention can be used to remove approximately 20 g of urea in a given treatment, in which at least 1.5 L of sweat are secreted (over 5-hour treatment period). The phosphate and magnesium concentrations in sweat are also higher than in serum. Accordingly, clearance of such components can also be adequately achieved with the systems and methods of the present invention. Therefore, systems and methods of the present invention can be of great benefit to patients currently on dialysis, that have irregular visits and to have an option for a home treatment, or if they are travelling and must skip the dialysis. The systems and methods of the present invention offers the flexibility of temporary treatments. Similarly, pre-dialysis patients may want to postpone the start of their dialysis treatment, and, as such, can utilize the systems of the present invention as a means of delaying the dialysis start.

### Clinical Study(ies):

A clinical study was conducted to study the feasibility and short-term performance of a novel approach to remove fluids and sodium directly from the interstitial compartment by enhancing sweat rate by using the systems and methods of the present invention. The study was performed in two phases: Phase I included 6 normal subjects, each undergoing three treatment sessions; and, after reviewing the safety data from the normal subjects, approval was obtained to proceed with phase II of the study, which included 10 patients with heart failure and evidence of fluid overload.

### Inclusion Criteria:

We recruited ambulatory patients with New York Heart Association (NYHA) class II or III with evidence of congestion (peripheral edema +2 or more), age ≥18 years, estimated glomerular filtration rate >15 ml/min/1.73 m², on a standing dose of ≥40 mg of furosemide per day, and with NT-pro BNP >300 pg/mL. Patients were excluded if they had any recent episodes of clinical decompensation, lower extremity skin conditions (open wounds, ulcers), or severe peripheral arterial disease.

### The Device:

The fluid stimulation system of the present invention is designed to enhance fluid and salt loss via the eccrine sweat glands. The system is comprised of two main functional units: (1) a capsule (also referred to as treatment device) and (2) heating units with controller. Additional temperature and humidity sensors are located within the capsule and on the patient's body, monitoring skin and ambient temperature.

The capsule is a loose body contacting cloth (Neoprene) chamber, designed to accommodate the patient's body from the foot and the entire torso, in standing, sitting or supine positions. The capsule has two hot air inflow ports on each side of the capsule's lower region and two hot air outflow ports on each side of the upper region. The capsule fits loosely around the patient such that once warm air moves over the entire body surface area enclosed by the capsule and can create a gap between the body surface and the capsule measuring 1-3 cm.

The capsule creates a homogeneous warm temperature environment around the lower part of the body leading to increased skin temperature that initiates perspiration. The system ensures, however, that the body core temperature remains within normal range. In addition, because patient's comfort is paramount with this form of therapy, the sweat evaporates instantaneously, thus avoiding the awareness of perspiration by the patient and enabling long durations of treatments, if required.

The heating subunit controls temperature elevation inside the capsule. The warm air is directed from the two heating units into the capsule in order to create a warm (temperatures ranging from 35-45° C.) and relatively dry environment around the patient from lower chest to feet, at relative humidity lower than 50% and flow rates of ˜1.5 m³/min. The skin temperature is uniformly increased from 32-33° C. to the range of 36-38° C., where the slope of the relationship between temperature and sweat production is linear, and discomfort or thermal injury does not occur.

A single temperature sensor is placed on the patient's skin in a location per physician discretion and provides a continuous temperature display for purposes of monitoring. Two relative humidity and temperature sensors are placed at the inflow and outflow ports which enable continuous estimation of sweat rate (see below). During device operation, any water appearing on the skin surface within the capsule evaporates immediately into the airstream. Therefore, a change in the humidity of the airstream is the result of sweating within the capsule and is detected by the humidity sensor. An additional sensor measures airflow rate.

### Study Protocol:

All normally prescribed HF medications, with the exception of diuretics, were taken in the morning, ˜2-3 hours prior a treatment session; diuretics were withheld. Each procedure lasted for up to 4 hours. Skin temperature was measured continuously. Core temperature was measured orally every 30 min. Serum creatinine, blood urea nitrogen, serum sodium and urinary sodium were measured prior to and after the procedure. Three treatment procedures were performed in each participant with 4-10 days between each procedure. Fluid intake and urine output were recorded during the procedure. The capsule was cleaned and sterilized after each treatment session.

### Study End Points:

The primary efficacy end point of the study was the ability to activate the fluid stimulation system and increase skin temperature to between 33° C. and 38° C., with subjects tolerating treatment for at least 2 hours. Secondarily, we evaluated efficacy of the system to produce a clinically meaningful hourly sweat output (weight loss due to increased sweating). We used a cutoff of ≥150 mL/h as a success criterion, based on the accepted satisfactory urine output in response to intravenous diuretics.

The primary safety endpoint was an assessment of major adverse cardiac events related to the procedure during the treatment periods and at up to 1-week follow-up after the last treatment session. An adverse event was defined as an increase in core temperature above normal range (37.5° C.), any skin injury (Rutherford classification stage 1 and above), an increase in core temperature above normal levels, worsening renal function, hypotension, or a clinically significant electrolyte abnormality.

To explore the possibility of monitoring treatment progress we also estimated sweat rate based on the differences between the inlet and outlet absolute humidity values (calculated from the measured relative humidity and temperature parameters) and the airflow rate using the following formula:

$$Sweat rate ( \frac{g}{min} ) = Air Flow ( \frac{m 3}{min} ) \times Absolute humidity inflow outflow difference ( \frac{g}{m 3} )$$

The calculated sweat rate was compared to the measured weight loss.

### Statistical Analysis:

Categorical variables are presented as frequencies. Continuous variables are presented as mean =SD or median with interquartile range (IQR). The normality of continuous variables was determined using the Shapiro-Wilk test.

Differences in continuous variables between different time points were analyzed using paired-sample Student 1-tests (accounting for clustering of standard errors at the session level) or Wilcoxon rank sum tests (for within-group differences), and the Mann-Whitney U test (for between-group differences).

For the comparison of weight loss at repeated treatment sessions, a mixed model was fit with residual maximum likelihood and small sample adjustment made using the Kenward-Roger method. To assess the agreement between weight loss and the estimated sweat rate, we calculated Lin's concordance class correlation (CCC) and constructed Bland-Altman plots.

The relation between the sweat rate and procedure time was estimated with the use of restricted cubic splines (with 3 knots placed at default locations), which allowed us to explore nonlinear relationships. Differences were considered statistically significant at the 2-sided P<0.05 level. Statistical analyses were performed using the Stata version 16.0 (College station, TX).

### Results:

Six normal subjects (mean age, 46±11 years; 2 female) and 10 patients with HF (mean age, 70±14 years; all male) were recruited. Of these, all normal subjects and 8 patients completed the planned protocol of 3 treatment sessions with the fluid stimulation system of the present invention. Two of the HF patients withdrew from the study after receiving a single treatment, wishing not to return to the hospital due to concerns related to the COVID-19pandemic. One additional patient was excluded after being screened and consented due to worsening renal function and clinical decompensation prior to treatment initiation.

The clinical characteristics of the 10 treated patients are listed in Table 3, provided below:

TABLE 3
Characteristics of the Heart Failure Patients

| Characteristic | Value | |
|---|---|---|
| Age | 70 ± 14 | |
| Hypertension | 9 (90%) | |
| Diabetes | 5 (50%) | |
| NYHA functional class | 2.7 ± 0.6 | |
| Ischemic etiology | 6 (60%) | |
| Atrial fibrillation | 7 (70%) | |
| ICD/CRT | 3 (30%) | |
| Baseline creatinine (mg/dl) | 1.06 | [0.93-1.37] |
| Estimated GFR (ml · min−1/1.73 m2) | 73 | [52-95] |
| NT-proBNP (ng/mL) | 602 | [427 to 1719] |

| | |
|---|---|
| LVEF (%) | 47 ± 16 |
| Mitral regurgitation ≥ Moderate | 4 (40%) |
| Pulmonary artery systolic pressure (mm Hg) | 38 ± 14 |
| Beta blockers | 6 (60%) |
| ACE Inhibitors/ARB | 7 (70%) |
| MRA | 9 (90%) |
| Daily furosemide does (mg) | 110 ± 37 |

The mean treatment duration of treatment sessions was 3.9 h. Operation of the device elevated mean skin temperature from baseline values of less than 34° C. to a median of 37.5° C. (IQR 37.1 to 37.9° C.) (FIG. 90). During the procedure, the core temperature remained within the normal range: median 36.8° C. (IQR 36.7 to 36.9° C.). The median change (averaged across all timepoints) in core temperature compared with baseline was 0.2° C. (IQR 0 to 0.3° C.; fF).

The median total weight change during the treatment (adjusted for urine produced and fluids consumed during the procedure), was 720 g (IQR 570 to 760 g) in the normal volunteers and 935 g (IQR 650 to 1060 g) in the HF patients (FIG. 91), with a mean of 219±67 g/lh of treatment (range 100 to 338 g/h). Sweat rate was ≥150 mL in 77% of treatment procedures the average. There were no differences in weight loss between HF patients and normal subjects (P=0.09). In a mixed repeated measures model, there were no significant between-treatment differences in the magnitude of weight loss within individual subjects over the 3 treatment sessions (P=0.47).

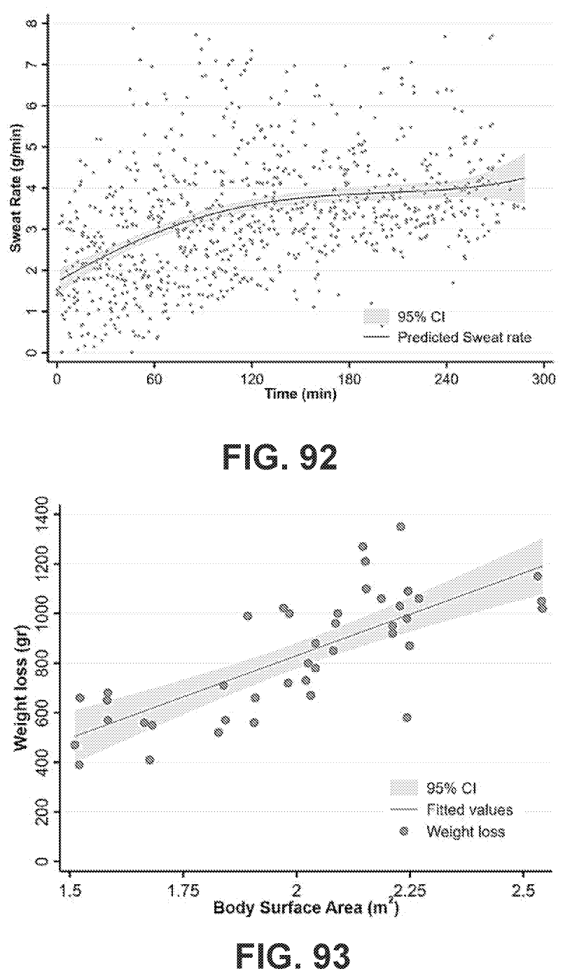

Using the whole study population, weight loss during the first 2 h of treatment (median 300 g, IQR 200 to 450 g) was lower than weight loss during the final 2 h of treatment (median 450 g, IQR 360 to 600 g; P-0.003). Cubic spline regression demonstrated that the mean sweat rate increased linearly, reaching a plateau after 2 h of treatment (FIG. 92).

Systolic (P=0.25) and diastolic (P=0.48) blood pressure and heart rate (P=0.11) remained unchanged during the procedure. The changes in renal function are shown in Table 4 provided below:

TABLE 4
Renal Function and Electrolytes

| | Baseline | End of Treatment | P Value |
|---|---|---|---|
| Creatinine (mg/dL) | 1.25 ± 0.46 | 1.27 ± 0.45 | 0.58 |
| BUN (mg/mL) | 33 ± 17 | 32 ± 17 | 0.003 |
| Serum Na (mmol/L) | 138 ± 2 | 139 ± 2 | 0.003 |
| Urine Na (mmol/L) | 47 [39-61] | 41 [20-59] | 0.21 |

Serum BUN and serum sodium showed no clinically significant difference (albeit statistically significant). Urinary sodium remained unchanged during the procedure.

Weight loss during the procedure was not affected by renal function. Study participants with estimated GFR below and above median (73 ml·min⁻¹/1.73 m²) had a similar weight loss (850 g [IQR 615-1011 g] vs. 833 g [IQR 578-1035 g] respectively; P-0.96) (Supplemental FIG. 91). There was a significant positive correlation between the calculated body surface area and sweat volume during the procedure (r-0.75; P<0.0001, FIG. 93).

### Measured Humidity and Weight Loss:

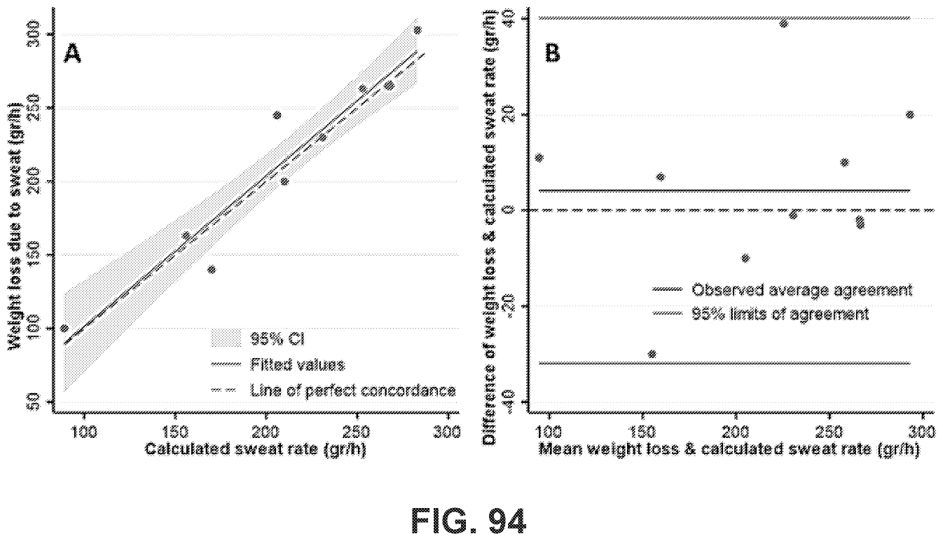

The calculated sweat rate based on the differences between the inlet (mean 14.4±1.4 g/m3) and outlet (mean 16.4 +1.8 g/m3) absolute humidity values and the airflow rate had a strong agreement with the sweat weight loss (CCC 0.88; 95% 0.78 to 0.98; FIG. 94A). However, Bland-Altman plots show that the limit of agreement ranged from −32 to 40 g/h (mean difference=4.2 g/h; FIG. 9B).

### Methods and Devices to Control Diuresis

Diuresis is a condition in which the kidneys filter too much bodily fluid, thereby increasing urine production and the frequency with which a person needs to urinate. In other words, diuresis involves extra urine production in the kidneys as part of the body's homeostatic maintenance of fluid balance. Many individuals with health issues, such as heart failure and kidney failure, require diuretic medications to help their kidneys deal with the fluid overload of edema. Such medications promote water loss via urine production. The concentrations of electrolytes in the blood are closely linked to fluid balance, so any action or problem involving fluid intake or output (such as polydipsia, polyuria, diarrhea, heat exhaustion, starting or changing doses of diuretics, and others) can require management of electrolytes, whether through self-care in mild cases, or with help from health professionals in moderate or severe cases. There is a known effect referred to as immersion diuresis, in which external pressure can be applied around a person's body (e.g., immersion of the body in water) and, as a result, the body reduces anti-diuretic hormone production, thereby cause a person to start to urinate. In particular, the external pressure constricts the veins, which results in the body detecting an increase in the blood pressure and inhibits the release of vasopressin (also known as antidiuretic hormone (ADH)), causing an increase in the production of urine.

The devices and methods of the present disclosure may be used to control diuresis. In particular, a wearable treatment device consistent with the present disclosure may be used to apply pressure to a patient based on adjusting air inflow and outflow (via the respective inlet and outlet(s) of the device and console unit for supply air to the device). More specifically, the device can be configured to receive 1.5 cubic liters per minute against a pressure gradient of 20 mm Hg, in which between 2 mmHg and 20 mmHg are applied to cause the immersion diuresis effect on the patient. It should be noted that, as opposed to relying on inducing sweat production with the use of a warm air environment, the wearable treatment device of the present invention simply provides an air-tight environment around the patient of about 20 mmHg, thereby imposing an external pressure thereupon and causing an immersion diuresis effect. As such, the air supplied to the device does not need to be heated or humidified and can even include cold air, so light as an environment having a positive pressures is provided within the device to initiate immersion diuresis phenomena, thereby resulting in urination.

Accordingly, the sensors associated with such a device are able to monitor a pressure within the environment created in the device and further control air inflow and outflow to maintain a constant desired pressure (e.g., controlling valves and the like and airflow into the device). It should be noted that the device does not need to be airtight, but rather sufficiently sealed and able to be pressurized when needed.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

1. A fluid stimulation system comprising:
a wearable treatment device to provide a warm air environment at a controlled air volume flow rate around a body part of a patient to shift fluids directly and non-invasively from an interstitial compartment of the patient to skin of the patient resulting in controlled fluid loss in the form of sweating;
a console unit operably coupled to the treatment device and configured to provide at least air to the treatment device; and
a computing device configured to communicate with the console unit and control operation thereof.

2. (canceled)

3. The system of claim 1, wherein the console unit comprises a controller communicatively coupled with a plurality of sensors that provide feedback data to the controller, the controller comprising a processor coupled to memory containing instructions executable by the processor to cause the controller to monitor and control conditions of the treatment device based, at least in part, on the feedback data provided from the plurality of sensors.

4. (canceled)

5. The system of claim 3, wherein the computing device provides a user interface operable by a user to monitor conditions of the treatment device and conditions of the patient during operation of the treatment device and console unit.

6. (canceled)

7. (canceled)

8. (canceled)

9. The system of claim 3, wherein the console unit comprises at least one of an air fan and a heat source for providing air or heated air to the treatment device, the console unit further comprising a flow meter associated with the air fan.

10. The system of claim 9, wherein the flow meter is communicatively coupled with the controller.

11. The system of claim 3, further comprising a skin temperature sensor positioned within a portion of the treatment device and configured to collect skin temperature data of the patient, the skin temperature sensor being communicatively coupled with the controller.

12. The system of claim 11, wherein the controller is configured to stop operation of the system if the skin temperature exceeds a threshold value, wherein the threshold value is less than 40° C.

13. (canceled)

14. The system of claim 3, further comprising at least one accelerometer sensor positioned within a portion of the treatment device and configured to collect acceleration data of the patient, the accelerometer sensor being communicatively coupled with the controller.

15. The system of claim 14, wherein the controller is configured to determine a patient's pose based, at least in part, on data received from the at least one accelerometer, wherein the patient's pose is selected from the group consisting of a supine or prone position, a sitting position, and a standing position.

16. (canceled)

17. The system of claim 3, wherein sweating is stimulated by controlling at least one of relative humidity, airflow, pressure, and temperature within the treatment device.

18. The system of claim 17, wherein the controller is configured to adjust output of at least one of air and heated air from the console unit to thereby control at least one of relative humidity, airflow, pressure, and temperature within the treatment device based on feedback data received from the plurality of sensors.

19. The system of claim 18, wherein skin temperature and sweat rate of the patient is controlled by the controller closing a control loop inputting relative humidity at an inflow and an outflow associated with the treatment device, an air flow rate, and skin temperature, and changing the flow rate and inflow air temperature until a sufficient sweat rate is achieved.

20. The system of claim 18, wherein the console unit is configured to adjust output of at least one of air and heated air therefrom based, at least in part, on an AI-based algorithm.

21. The system of claim 20, wherein the console unit is configured to obtain and process feedback data received from the plurality of sensors, wherein the feedback data comprises at least one of skin temperature, ambient temperature, core patient temperature, blood pressure, heart rate, patient acceleration, sweat rate at the ambient temperature, and sweat rate at the skin temperature.

22. The system of claim 21, wherein the feedback data is processed using a machine learning system, wherein the machine learning system comprises a neural network trained to identify optimal working parameters of the system with respect to a given patient undergoing treatment for fluid stimulation.

23. (canceled)

24. The system of claim 22, wherein the controller is configured to adjust output of air and/or heated air therefrom based on the identified optimal working parameters so as to achieve an optimal warm air environment around the body part of the patient.

25. The system of claim 22, wherein processing of the feedback data comprises correlating the feedback data with reference data, wherein the reference data comprises prior treatment data of the patient.

26. (canceled)

27. (canceled)

28. (canceled)

29. (canceled)

30. The system of claim 1, wherein the treatment device comprises a wearable device configured to cover one or more body parts of the patient to undergo treatment, wherein the wearable device comprises an outer layer of material and an inner layer of material cooperatively forming a chamber therebetween configured to receive air from the console unit.

31. (canceled)

32. The system of claim 30, wherein the outer and inner layers are joined to another via a plurality of separate fixation points.

33. The system of claim 32, wherein the outer and inner layers are joined to another via fabric welding.

34. (canceled)

35. The system of claim 30, wherein the inner layer comprises a plurality of apertures allowing air to flow from the chamber through the inner layer and provide the warm air environment at a controlled air volume flow rate around a body part of a patient wearing the wearable device.

36. The system of claim 30, wherein the wearable device further comprises a plurality of spacing members provided between the outer and inner layers at a rear portion of the wearable device corresponding to, and fitting over, a posterior portion of the patient.

37. The system of claim 36, wherein each of the plurality of spacing members is configured to maintain air flow within the chamber at the rear portion of the wearable device when the patient is in either of a supine position or seated position when wearing the wearable device.

38. The system of claim 30, wherein the wearable device further comprises a rear spacer member positioned on an interior surface of the inner layer at a rear portion of the wearable device corresponding to, and fitting over, a posterior portion of the patient.

39. The system of claim 38, wherein the rear spacer member is configured to contact a posterior portion of the patient and maintain separation of the interior surface of the inner layer from the posterior portion of the patient when the patient is in either a supine position or seated position.

40. The system of claim 39, wherein the rear spacer member comprises a plurality of channels and apertures configured to maintain adequate air flow within the warm air environment surrounding the patient when the patient is in either a supine position or seated position.

41. (canceled)

42. (canceled)

43. (canceled)

44. (canceled)
